(19) United States
(12) Patent Application Publication
(10) Pub. No.: US 2014/0081579 A1
(43) Pub. Date: Mar. 20, 2014

(54) GREENHOUSE GAS GRID AND TRACKING SYSTEM

(71) Applicant: CARBON AUDITORS INC., Hydes, MD (US)

(72) Inventor: Matthew G. Tyburski, Hydes, MD (US)

(73) Assignee: CARBON AUDITORS INC., Hydes, MD (US)

(21) Appl. No.: 13/965,302

(22) Filed: Aug. 13, 2013

Related U.S. Application Data
(62) Division of application No. 12/847,370, filed on Jul. 30, 2010, now abandoned.
(60) Provisional application No. 61/230,235, filed on Jul. 31, 2009.

(51) Int. Cl. G01N 33/00

(52) U.S. Cl. CPC G01N 33/0004
USPC 702/19

(57) ABSTRACT

A method and computer system for reporting on a target greenhouse gas within a geographical boundary of an offset project by compiling policy parameters for the target greenhouse gas and generating a science plan for monitoring the target greenhouse gas for the target geographical boundary of the offset project, based upon the compiled policy parameters. An allometric model for the target greenhouse gas within the geographical boundary of the offset project is generated based upon the science plan of the target greenhouse gas for the geographic boundary, and a report for the target greenhouse gas within the target geographical boundary of the offset project is generated based upon the allometric model.

### CROSS-REFERENCE TO RELATED APPLICATIONS

This application is a U.S. Divisional patent application of U.S. patent application Ser. No. 12/847,370, filed Jul. 30, 2010 and claims priority to U.S. Provisional Patent Application No. 61/230,235, filed Jul. 30, 2009 entitled Greenhouse Gas Monitoring Grid For Terrestrial Carbon Credits, by Matthew G. Tyburski, the contents of such applications being incorporated by reference herein.

### COPYRIGHT NOTICE

A portion of the disclosure of this patent document contains material to which a claim for copyright is made. The copyright owner has no objection to the facsimile reproduction by anyone of the patent document or the patent disclosure, as it appears in the Patent and Trademark Office patent file or records, but reserves all other copyright rights whatsoever.

### BACKGROUND OF THE INVENTION

1. Field

The described embodiments relate to monitoring and reporting of greenhouse gases (GHGs).

2. Description of the Related Art

A sector in the green economy is the trade in greenhouse gases (GHGs), for example, carbon gas, and this sector can be referred to generally as “GHG (e.g., carbon) trading.” Using carbon as an example of a greenhouse gas, carbon emissions and offsets are traded under carbon trading mechanisms. Currently, there are both regulated and voluntary carbon trading mechanisms. A carbon trading mechanism is a legal trading scheme or standard that acknowledges certain activities as a carbon credit. One sector in carbon trading is developing carbon offsets from terrestrial (i.e., land-based) carbon sequestration and storage. Carbon is sequestered and stored by plants and/or vegetation. Under certain carbon trading mechanisms, the carbon that is sequestered and stored in plants or vegetation can be monetized as an offset through credible anthropogenic activities. One such activity is known as afforestation, reforestation and/or re-vegetation and involves the human assisted planting of trees to reduce atmospheric GHGs by carbon sequestration. Another credible activity through certain carbon trading mechanisms is known as Reduced Emissions from Deforestation and Degradation (i.e., REDD). REDD relates to the protection, preservation and/or conservation of carbon stored in trees through activities that avoid future potential GHG emissions from deforestation and/or degradation.

### SUMMARY OF THE INVENTION

It is an aspect of the embodiments discussed herein to provide an effective and efficient monitoring and reporting of any greenhouse gas, for example, one or more carbon based chemical elements, through an offset activity. According to an aspect of an embodiment, any GHG offset activity related to sustainable and/or improved management of an eco-region(s) (e.g., land, agriculture, water, species), that also reduces and/or removes emissions can be monitored and reported.

The above aspects can be attained by a method and computer system for reporting on a target greenhouse gas within a geographical boundary of an offset project, by compiling policy parameters for the target greenhouse gas; generating a science plan for monitoring the target greenhouse gas for the target geographical boundary of the offset project, based upon the compiled policy parameters; generating an allometric model for the target greenhouse gas within the geographical boundary of the offset project, based upon the science plan of the target greenhouse gas for the geographic boundary, and generating a report for the target greenhouse gas within the target geographical boundary of the offset project based upon the allometric model.

These together with other aspects and advantages which will be subsequently apparent, reside in the details of construction and operation as more fully hereinafter described and claimed, reference being had to the accompanying drawings forming a part hereof.

### BRIEF DESCRIPTION OF THE DRAWINGS

FIG. 1 shows a summary of the full software process.

FIG. 2A shows the generic process used to develop a legal/policy analysis.

FIG. 2B shows the process used to retrieve text from legal/policy documents.

FIG. 2C shows the process used to develop a structured analysis for legal/policy documents.

FIG. 2D shows the generic process for the legal/policy analysis from FIG. 2a applied to voluntary mechanisms.

FIG. 3 shows a generalized flowchart of the carbon cycle defined by the IPCC.

FIG. 4A shows the process used to retrieve text from the database for current and planned satellite missions.

FIG. 4B shows the output from the assessment of a satellite sensor's data continuity for monitoring the project lifetime of an offset project site.

FIG. 4C shows the process used to retrieve text from publications for relevant science on monitoring vegetation attribute(s) with the identified satellite instrument.

FIG. 4D shows the generic process used to develop a science plan.

FIG. 4E shows the generic process used to develop a science plan applied to examples for monitoring vegetation growth and stocks with MODIS imagery.

FIG. 5A shows an example of the process used to develop Allometric Equations 1.

FIG. 5B shows an example of the process used to implement Allometric Equations 1 with remote sensing imagery.

FIG. 5C shows an example of the process used to develop Allometric Equations 1.

FIGS. 5D-1 and 5D-2 show an example of the process used to develop Allometric Equations 2.

FIG. 6A shows the process used to develop the geospatial database and the contents that are stored on it.

FIG. 6B shows an example of a georeferenced file and a file with a gridcode.

FIG. 7A shows the generic process for pre-processing the raw remote sensing imagery stored in the primary database.

FIG. 7B shows an example of the generic process for pre-processing the raw remote sensing imagery stored in the primary database.

FIG. 8A shows the generic process to develop Allometric Equations 1.

FIG. 8B shows an example for the generic process to develop Allometric Equations 1.

FIG. 9A shows the first half of the generic process to develop Allometric Equations 2.

FIG. 9B shows an example for the first half of the generic process to develop Allometric Equations 2.

FIG. 9C shows examples for sampling a remote sensing imagery for a client's vector file with samples of vegetation attribute(s).

FIG. 10A shows the second half of the generic process to develop Allometric Equations 2.

FIG. 10B shows an example for the second half of the generic process to develop Allometric Equations 2.

FIG. 10C shows the illustrative outputs from developing Allometric Equations 2 from a Random Forest training model.

FIG. 10D shows the text outputs from developing Allometric Equations 2 from a Random Forest training model.

FIG. 11A shows the generic process for implementing Allometric Equations 1.

FIG. 11B shows an example for the generic process used for implementing Allometric Equations 1

FIG. 11C shows atlases used in implementing Allometics 1 in geospatial data processing software.

FIG. 12A shows the generic process for implementing Allometric Equations 2 with remote sensing imagery.

FIG. 12B shows an example for the generic process for implementing Allometric Equations 2 with remote sensing imagery.

FIG. 12C shows an example of the outputs of Allometric Equations 2 in a spreadsheet.

FIG. 12D shows an example of the mapped outputs from converting the spreadsheet output in FIG. 12C to geospatial data.

FIG. 13A shows the generic process used to obtain a digital boundary for a project site from a client.

FIG. 13B shows an example for the generic process used to obtain a digital boundary for a project site from a client.

FIG. 14A shows the generic process used to sample the client's digital boundary for a vegetation attribute and other geospatial data stored on the databases.

FIG. 14B shows an example for the generic process used to sample the client's digital boundary for a vegetation attribute and other geospatial data stored on the databases.

FIG. 14C shows an example of the process used to sample the geospatial data for a vegetation attribute with a client's digital boundary.

FIG. 15 shows the generic process used to develop a report for a client.

FIG. 16A shows the Central Database.

FIG. 16B shows the assembly for the final report.

FIG. 17 is a functional block diagram of a computer for the embodiments of the invention.

### DETAILED DESCRIPTION OF THE PREFERRED EMBODIMENTS

It is an aspect of the embodiments discussed herein to provide an effective and efficient monitoring and reporting of any Green House Gas (GHG), for example, one or more carbon based chemical elements, in a credible offset activity under a trading mechanism. According to an aspect of an embodiment, any GHG offset activity related to sustainable and/or improved management of an eco-region(s) (e.g., land, agriculture, water, species), that also reduce and/or remove emissions can be monitored and reported.

The embodiments are described by referring as an example to carbon and monitoring of one or more vegetation attributes as a carbon offset activity, however, the embodiments are not limited to carbon and carbon offset activities, but other GHGs and corresponding eco-region offset activities can be monitored and reported under any trading scheme, standard and/or tax system.

The embodiments referring to the example of vegetation attributes define a GHG as the composition of one or more chemical element(s) (e.g., carbon) that are manifested in the physical property and/or structure of a plant and/or vegetation and are required for monitoring and/or reporting of an offset activity under any trading scheme. This definition for GHGs in the embodiments is not limited to vegetation, however, and can mean any composition of one or more biogeophysical elements(s) for monitoring an eco-region that are required for monitoring and/or reporting of an offset activity under any trading scheme, standard and/or tax system.

Using carbon offset trading as an example, a project developer in carbon trading is a legal entity that intends to develop a carbon offset project. Project developers gain accreditation for a project in which they intend to develop as a carbon offset. Trading mechanisms accredit projects. The project developer will sell an accredited carbon offset credit to a polluter who is a legal entity that emits carbon through an industrial activity (i.e., a “carbon footprint”). The purchase of the credible carbon offset by a polluter means the polluter's carbon footprint is reduced and/or null (i.e., “carbon neutral”). Carbon trading mechanisms provide guidance on how a project developer must manage land and comply with monitoring and reporting of GHG emissions and removals at the accredited project site. Information on GHG emissions and removals at a project site is required for fulfilling the monitoring and reporting requirements for the carbon trading mechanism.

For example, the monitoring of GHG emissions and removals is reported in project design documents, project validation documents and project verification documents required for compliance and crediting for an accredited project under the carbon trading mechanism. This patent relates to a software process, executed by a computer, for monitoring GHG offset activities, for example, terrestrial carbon sequestration and storage, to fulfill the compliance guidelines under trading mechanisms relevant to the client. The monitoring information for carbon sequestration and storage is used by a client to monitor, track and/or report GHG emissions and removals at the accredited project site.

An example of the described process is for monitoring and reporting GHG emissions and removals at a project site to fulfill compliance under a relevant carbon trading mechanism. The process in this patent may also have incidental and/or collateral industrial utility for land owners and/or an entity that holds the legal title to the exploitation of natural resources at an eco-region, and/or other related industry that is seeking to appraise GHG emissions, removals and/or make assessments of other relevant vegetation attributes in a specific plot of land. The latter may also include the internal offsetting by a company. The process is distinguished from research that is of a purely philosophical pursuit, because fulfilling monitoring and reporting compliance under a trading mechanism is explicit in the process. Academic research is defined here as monitoring vegetation and/or the terrestrial environment without the explicit intent to comply with any and/or all trading mechanisms. Without intellectually embedding the process in compliance guidance for GHG (e.g., carbon) trading, the process would have no defined industrial application. Therefore, the process disclosed herein is novel as it builds the scientific method used for monitoring and reporting out of the legal requirements of a trading mechanism, rather than intellectually forcing a scientific method into the law. The final output of the process in this patent is, for example, a report that project developers of GHG offsets (e.g., terrestrial carbon offsets) use to show how their monitoring is compliant with the legal requirements when they report to a relevant trading mechanism.

According to an aspect of an embodiment, a method and system is provided for reporting on a target greenhouse gas within a geographical boundary of an offset project, by compiling policy parameters for the target greenhouse gas; generating a science plan for monitoring the target greenhouse gas for the target geographical boundary of the offset project, based upon the compiled policy parameters; generating an allometric model for the target greenhouse gas within the geographical boundary of the offset project, based upon the science plan of the target greenhouse gas for the geographic boundary, and generating a report for the target greenhouse gas within the target geographical boundary of the offset project based upon the allometric model.

According to another aspect of an embodiment, the following operations are provided: Step 1) Develop a Legal/Policy Analysis for a vegetation attribute, Step 2) Develop a Science Plan to monitor a vegetation attribute, Step 3) Develop a Geospatial Database to monitor a vegetation attribute, Step 4) Develop Allometric Equations to monitor a vegetation attribute, Step 5) Implement Allometric Equations with remote sensing imagery to monitor a vegetation attribute, Step 6) Obtain a Client's Boundary of a project site, Step 7) Sample a Client's Boundary for a project site with the geospatial data for a vegetation attribute, Step 8) Develop a Report describing a vegetation attribute at a client's project site.

Specifically, Step 1) is developed from the legal requirements for monitoring a vegetation attribute relevant to a client. Step 1) can be updated and/or edited on the content for monitoring a vegetation attribute with updates, changes and/or revisions to existing law, for new law, for new clients and between different projects that may have different requirements for monitoring a vegetation attribute. Step 2) for developing a Science Plan is completed with input from Step 1) and is dependent on the outputs from Step 1). This means that the Science Plan can be edited, changed, updated, and/or revised with changes to the content of Step 1). Step 3) for developing a Geospatial Database is completed with input from Step 2) and is dependent on the outputs from Step 2). This means that the geospatial data used to develop a Geospatial Database in Step 3) can be edited, changed, updated, and/or revised with changes to the content of Step 2). Step 4) for developing Allometric Equations is completed with input from Step 2) and is dependent on the outputs from Step 2). This means that the methods used to develop Allometric Equations in Step 4) can be edited, changed, updated, and/or revised with changes to the content of Step 2). Step 5) for implementing Allometric Equations with remote sensing imagery is completed with input from Step 2) and is dependent on the outputs from Step 2) and Step 4). This means that the methods used to implement Allometric Equations with remote sensing imagery in Step 5) can be edited, changed, updated, and/or revised with changes to the content of Step 2) and Step 4). Step 6) for obtaining a Client's Boundary for a project site is applicable to any boundary in a geospatial data file obtained from any client. Step 7) for sampling the Client's Boundary for a project site is dependent on the geospatial data used to develop a Geospatial Database in Step 3), the outputs for implementing Allometric Equations with remote sensing imagery in Step 5) and the project site Boundary File obtained from the client in Step 6). This means that the outputs for sampling a Client's Boundary in Step 7) can be edited, changed, updated, and/or revised with changes to the content in Step 3), Step 5) and Step 6). Step 8) for reporting a vegetation attribute to a client is dependent on aforementioned steps from Step 1) to Step 7). This means that the outputs for developing a Report for a vegetation attribute at a client project site in Step 8) can be edited, changed, updated, and/or revised with changes to the content in Step 1) through Step 7).

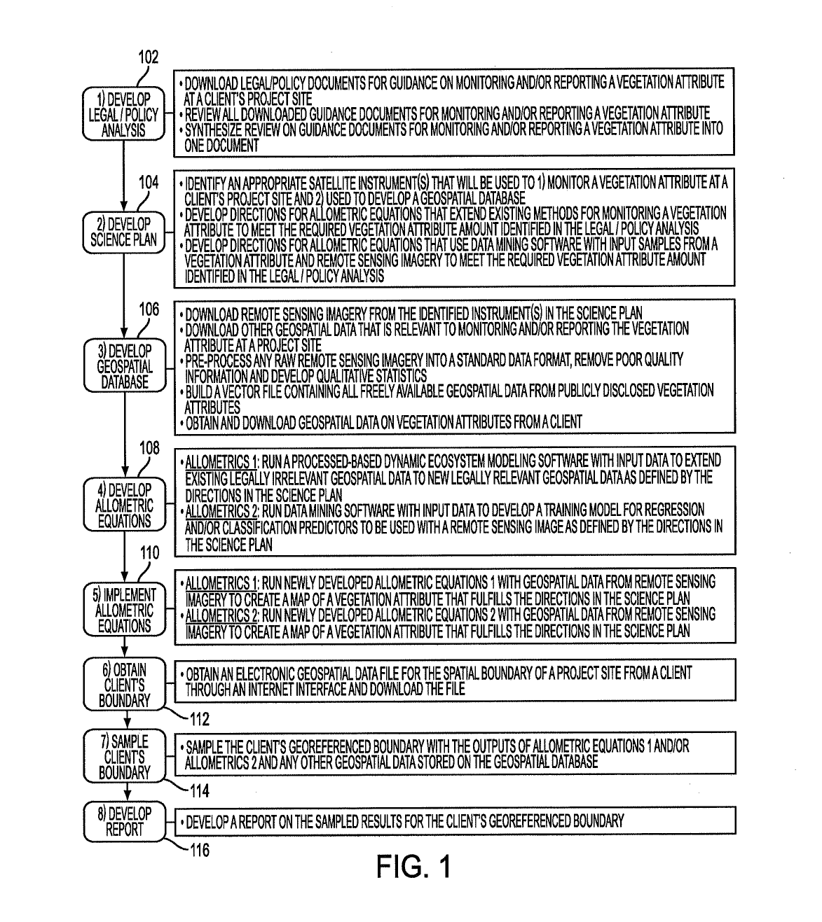

FIG. 1 shows the embodiment of the full process in more detail. In 102, a legal/policy analysis is developed for the monitoring and/or reporting for a vegetation attribute(s) under relevant GreenHouse Gas (GHG) trading schemes and/or standards to a client. The client will intend to monetize the vegetation attribute(s) at a project site(s) under the relevant GHG trading schemes and/or standards. GHG trading schemes and/or standards are also defined as a trading mechanism that provides a legal obligation between the project developer client and other legal entities. Examples of vegetation attributes that a client can monetize for activities at a project site are terrestrial carbon sequestration and storage. Documents related to guidance on monitoring and/or reporting vegetation attributes under the trading mechanism are stored on a database.

First, a review is completed on one or more (for example, all) documents related to international law and policy guidance on monitoring and/or reporting the relevant vegetation attribute. Second, a review is completed on documents related to monitoring and/or reporting under regulated trading mechanism(s) that support the monetization for the intended vegetation attribute(s) by the client. Third, a review is completed on documents related to monitoring and/or reporting under voluntary trading mechanism(s) that support the monetization for the intended vegetation attribute(s) by the client. The term “review” in this context refers to the use of text retrieval software and/or search technology to key word retrieve/search for relevant information on monitoring and/or reporting of a vegetation attribute from one or more target policies, for example, the three types of aforementioned legal/policy information (e.g., legal/policy databases, documents, etc.) related to the target vegetation attribute. A user and/or computer implemented synthesis is completed on the retrieved information. A report can then be assembled on a word processing program that first provides a top-down synthesis on one or more (for example, all) monitoring and/or reporting requirements for the vegetation attribute in documents with the most wide ranging legal implications to the trading mechanism relevant to the client. The report next provides a bottom-up summary on how the monitoring and/or reporting for the vegetation attribute in the specific trading mechanism relevant to the client is then related back up to documents with wider-ranging legal implications. The report is either stored in electronic media on a computer hard drive and/or is printed out in hard copy with printer. The report can be updated and/or edited to include revisions and/or updates to legal/policy documents and/or amended to include new legal/policy documents relevant to monitoring and/or reporting of a vegetation attribute by a client.

In 104 from FIG. 1, a science plan is developed from the output of 102. The science plan first develops a strategic review of current and future planned remote sensing instrument capabilities onboard satellite missions. The strategic review of remote sensing instruments combined with the knowledge obtained from the legal/policy review from 102 is used to identify an appropriate satellite sensor to monitor the client's vegetation attribute. An intelligence assessment is developed on the current knowledge base in peer-reviewed journal articles for methods and/or techniques in monitoring a vegetation attribute with the remote sensing instrument defined in the strategic review. The science plan next uses the information developed in intelligence assessment that define the current knowledge base in public access to define directions for methods and/or techniques that will be used to meet the monitoring and/or reporting requirements required by the client that are defined in the report for the legal/policy analysis in 102.

The new directions are developed by a user for two approaches to monitor a vegetation attribute with remote sensing imagery through the development and implementation of allometric equations (i.e., fractions, regressions and/or classification functions). An allometric model (also referred to as an allometric equation) with respect to a vegetation attribute as an example is defined as 1) using fractions to relate a biophysical element of a vegetation attribute to another biophysical element of a vegetation attribute and/or 2) using regression and/or classification functions to relate a physical measurement of a vegetation attribute (e.g., obtained from geospatial data for a targeted vegetation attribute) to digital information measurable in pixels of a remote sensing image.

According to an aspect of an embodiment, allometric equations 1 and/or 2 will be used to extend and/or build on existing methods and/or techniques that at present do not fulfill the monitoring requirements required by a client that are defined by the legal/policy analysis in 102. The basic concept of Allometric Equations 1 is to develop an extension of existing science to meet the monitoring and/or reporting requirements defined in 102. Allometric Equations 1 develop fractions learned from processed-based dynamic ecosystem modeling software that are implemented as an extension to existing remote sensing-derived methods for monitoring a vegetation attribute, that with the added extensions meet the requirements for monitoring and/or reporting identified in 102.

Allometric Equations 2 use data mining software with physical samples of vegetation attribute(s) collected from a target geographical boundary (e.g., the ground) and remote sensing imagery to develop predictive regression and/or classification functions that are implemented with a full remote sensing image(s) to meet the requirements for monitoring and/or reporting identified in 102. In 106, a database(s) is developed that is based on the information developed in the science plan. The directions to implement methods and/or techniques for monitoring a vegetation attribute contained in the science plan will be used in 108 and 110 to develop and implement the allometric equations with the geospatial database developed in 106. Therefore, the science plan is used as an intellectual bridge between what is required by the client to comply with relevant monitoring and/or reporting requirements for a vegetation attribute(s) defined by 102 and the following steps in 106 through 116 that develop and implement the monitoring of the vegetation attribute and report the sampled vegetation attribute at a project site to the client. The science plan is drafted on a word processing program as a report, stored in electronic media on a computer hard drive and/or is printed out in hard copy on a printer. The science plan report can be updated and/or edited to include revisions and/or updates to the legal/policy analysis report completed in 102.

In 106 from FIG. 1, a geospatial database is developed from the science plan that was developed in 104. The geospatial database is comprised of two types of data: 1) freely available geospatial data and 2) geospatial data that is purchased on behalf the client. Geospatial data is defined as data and information that are referenced to a location on the Earth's surface. The geospatial data is in either raster and/or vector file format. Remote sensing data is in raster file format. Vegetation attribute data can be either in raster and/or vector file format. The freely available geospatial data is downloaded from internet accessible archives and/or websites. Remote sensing imagery is from either satellite-borne active and/or passive sensors. Remote sensing imagery may also be from sensor instruments onboard an unmanned aerial vehicle. Freely available remote sensing imagery is downloaded and stored on the geospatial database. Freely available climate, elevation and soil data is downloaded and stored on the geospatial database. Freely available data for a vegetation attribute is downloaded and stored on the geospatial database. Peer-reviewed literature and trading mechanism reports that disclose geospatial data for vegetation attributes are downloaded and stored on the geospatial database. Official government disclosures of geospatial data for vegetation attributes are downloaded and stored on the geospatial database. Other freely available geospatial data can be downloaded and stored on the geospatial database at the request of the client and/or with updates to the science plan. In the case of geospatial data that is remote sensing imagery, the data can be pre-processed from the raw downloaded data in a number of ways to change the file storage type, remove poor quality information, and develop qualitative statistics. In the case of geospatial data that is of a vegetation attribute, whether in a text publication and/or in a geospatial data file format, the data can be converted to a new geospatial file that combines one or more (for example, all) geospatial data for the vegetation attribute into one file. Geospatial data for a vegetation attribute is obtained from the client via the internet, downloaded and placed into a unique geospatial data file that is confidential and only for use in monitoring activities for the client. Downloaded geospatial data and other relevant information in 106 are stored in electronic media on a hard drive. The contents of the geospatial database are dependent on the strategic review, intelligence assessment and directions contained in the science plan.

In 108 from FIG. 1, the two types of allometeric equations are mentioned that are developed by following the directions from science plan in 104. Allometric Equations 1 are developed to quantify a vegetation attribute(s) from a dynamic ecosystem modeling software that is stored on the hard drive and installed on a computer workstation. The dynamic ecosystem modeling software is processed with input geospatial data from the geospatial database in 106. The fractions developed for Allometric Equations 1 partition target vegetation attribute as 100 percent to other targeted vegetation attributes that are a fraction of the 100 percent. The new fractions are the output of Allometric Equations 1 in 108.

Allometric Equations 2 are developed with the data mining software to train a predictive model for an input physical sample of a vegetation attribute(s) with a sample of pixels from an input remote sensing image, where input samples are stored on the geospatial database. Input data from physical samples of vegetation attribute(s) have a geographical coordinate on the Earth's surface. A physical sample is defined as one or more of: 1) a geo-referenced sample for a vegetation attribute that was obtained on the ground (i.e., on the terrestrial surface of the earth); 2) any geospatial data for vegetation attribute(s) that was created from a ground sample(s) and is disclosed as a map in either a raster and/or vector file; 3) standard remote sensing products that use ground data to validate and/ verify the standard product. The point of the second two definitions for a physical sample is to data mine pre-existing geospatial data that is publically disclosed, has an associated peer-review publication and/or is an official government disclosure of a vegetation attribute, but the underlying mathematical process used to develop the publically disclosed geospatial data is not replicable by the user. Data mining this publically disclosed geospatial data is completed by extracting a mathematical function that will replicate an output with the input remote sensing data that is very similar (i.e., with a high value for the coefficient of determination) to the publically disclosed geospatial data. Input data from remote sensing imagery is a sample of digital pixel information from the remote sensing image(s) at the same geographical coordinate of each vegetation attribute. The input physical sample(s) for the vegetation attribute(s) are used as the target variable in data mining software. Target variable means the y-axis variable that is used as an actual sample to train the prediction model for input(s) variables on the x-axis in the data-mining software. The sample(s) from the remote sensing imagery are used as the input x-axis variable(s) that will be trained to predict the target y-axis variable in the data-mining software. The data mining software develops a predictive regression and/or classification training model between the geospatial samples for the target vegetation attribute and the pixel samples from the remote sensing image(s). The predictive training model is the output of Allometric Equations 2 in 108. The outputs of 108 are stored in electronic media on a hard drive and can be printed out on a printer.

In 110 from FIG. 1, Allometric Equations 1 and Allometric Equations 2 that were developed in 109 are implemented with remote sensing imagery and the directions contained in the science plan in 104. Allometric Equations 1 are implemented by processing existing remote sensing-derived vegetation attributes that do not meet the monitoring requirements for the vegetation attribute identified in 102. The implementation of Allometrics 1 for the newly developed extensions in 108 transform pre-existing information that is legally insignificant to monitoring and/or reporting requirements for a vegetation attribute indentified in 102 to new information about a vegetation attribute that is legally significant and matches the requirements for monitoring and/reporting identified in 102. Allometics 2 are implemented by first using the data mining software to process the geospatial data contained in the full remote sensing imagery with the predictive training model developed for the vegetation attribute in 108. The data mining software scores (i.e., models) the geospatial data/information in the full the remote sensing imagery with the predictive training model. The scoring (i.e., modeling) transforms the original digital geospatial data/information contained in the remote sensing image to new information that is a prediction for the vegetation attribute. The outputs from the data mining software are then converted to a geospatial map of the vegetation attribute. The outputs of Allometric Equations 1 and Allometric Equations 2 are new geospatial data about the vegetation attribute projected as a map. The outputs of 110 are stored in electronic media on a hard drive as a database and can be printed out on a printer.

In 112 from FIG. 1, an electronic geospatial data file for the geographical area and/or boundary of the client's project site is obtained through an internet interface (i.e., email and/or a website). The client's geospatial data file is downloaded from the internet interface and stored in electronic format on the hard drive.

In 114 from FIG. 1, geospatial data processing software is used to overlay the client's geospatial data file for a project boundary on the outputs of Allometric Equations 1 and/or Allometric Equations 2 from 110 and other geospatial data stored on the geospatial database developed in 106. The geospatial data processing software is next used to sample and/or clip the client's project boundary file for the outputs of Allometric Equations 1 and/or Allometric Equations 2 and any other geospatial data stored on the geospatial database developed in 106. The newly sampled outputs are stored in electronic format on a hard drive.

In 116 from FIG. 1, a report is developed from the outputs of steps 102 to 114. The report assembles the outputs of 102 through 114 into one document and provides a synthesis of the material in relation to monitoring the vegetation attribute at the client's project site. The report is drafted in electronic media with a word processing program. The report is stored in electronic format on a hard drive and/or printed in hard copy. The report is transmitted to the client electronically through an internet interface (i.e., email and/or a web-site).

The legal/policy analysis is completed to provide intellectual input to the science plan. If the science has no link with the legal compliance for monitoring and/or reporting under a trading mechanism, the science is merely a philosophical pursuit because it is without direction from a relevant trading mechanism, which in practice means that the science that is not informed by the law/policy may not provide a measurement of a vegetation attribute that is fungible with an emission unit under the law. Therefore, before any process related to monitoring and/or reporting can be conceived or implemented, the legal requirements defined in guidance documents must first be identified and defined.

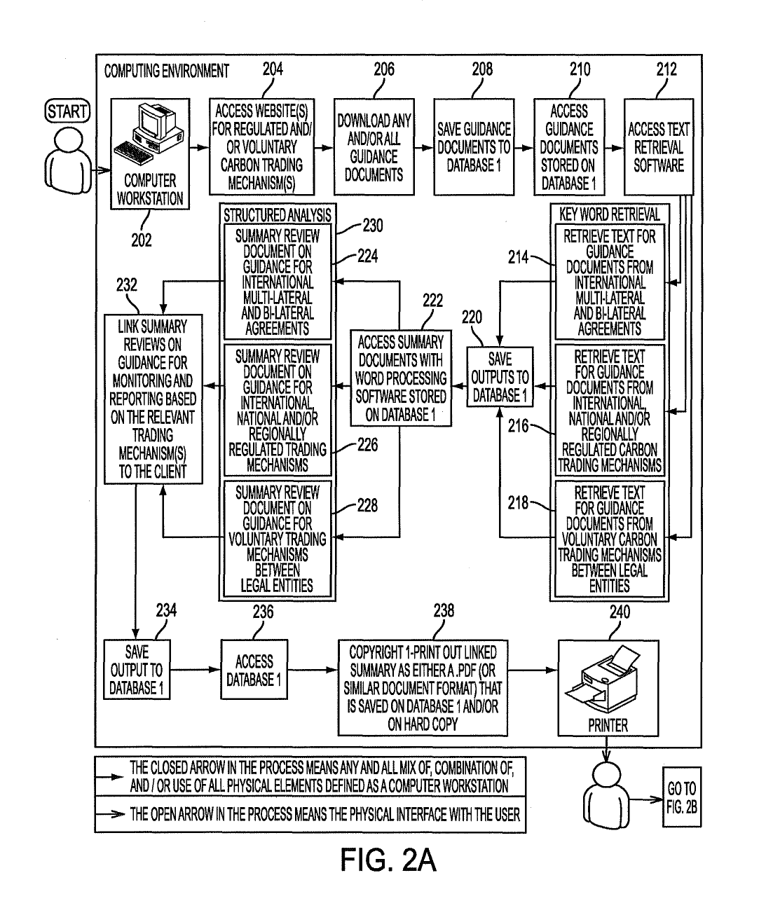

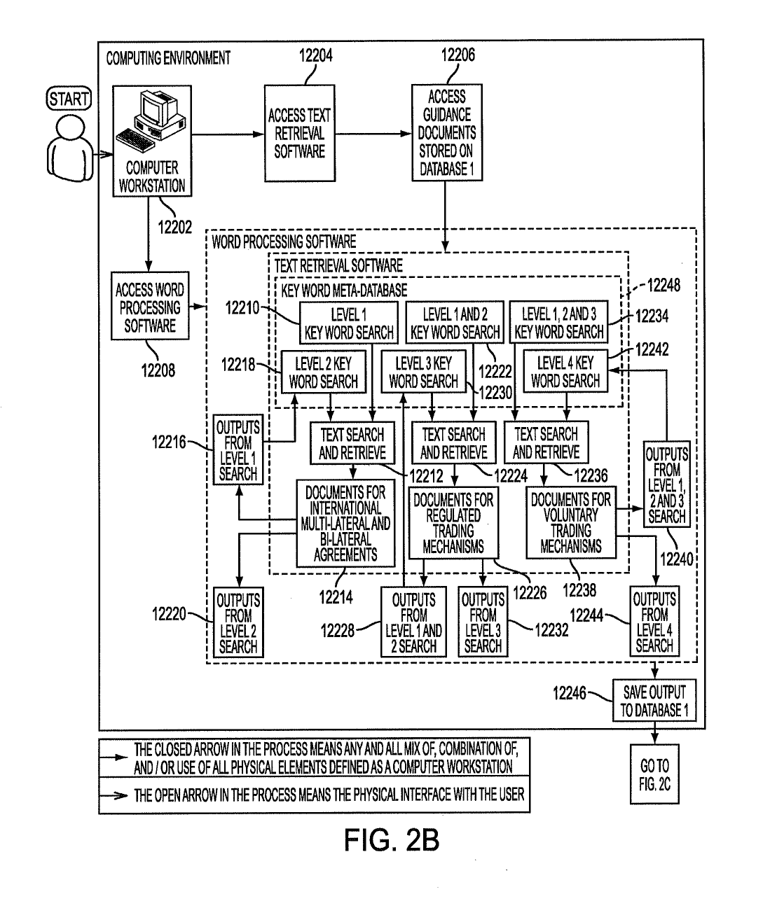

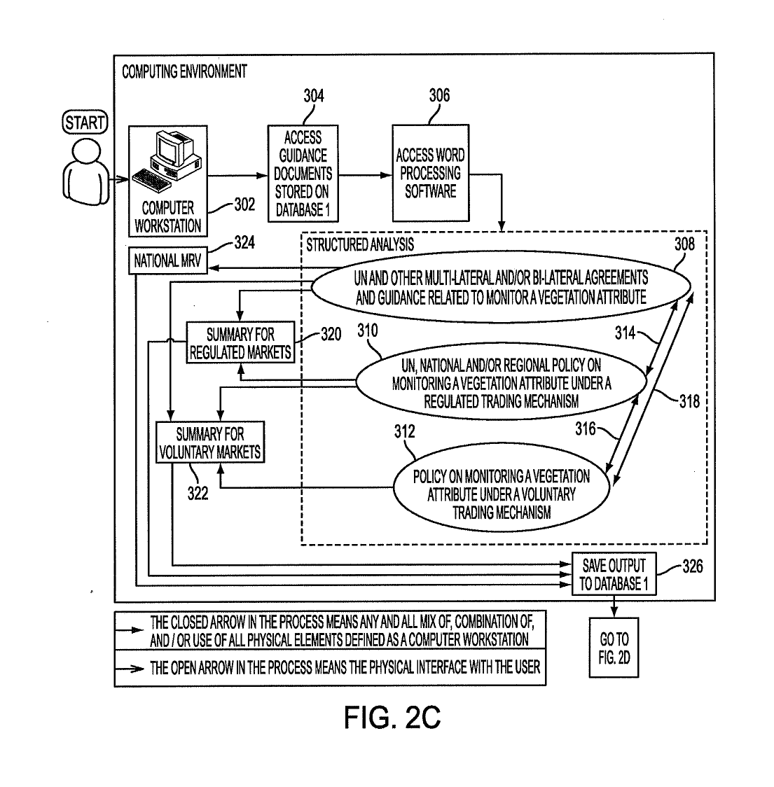

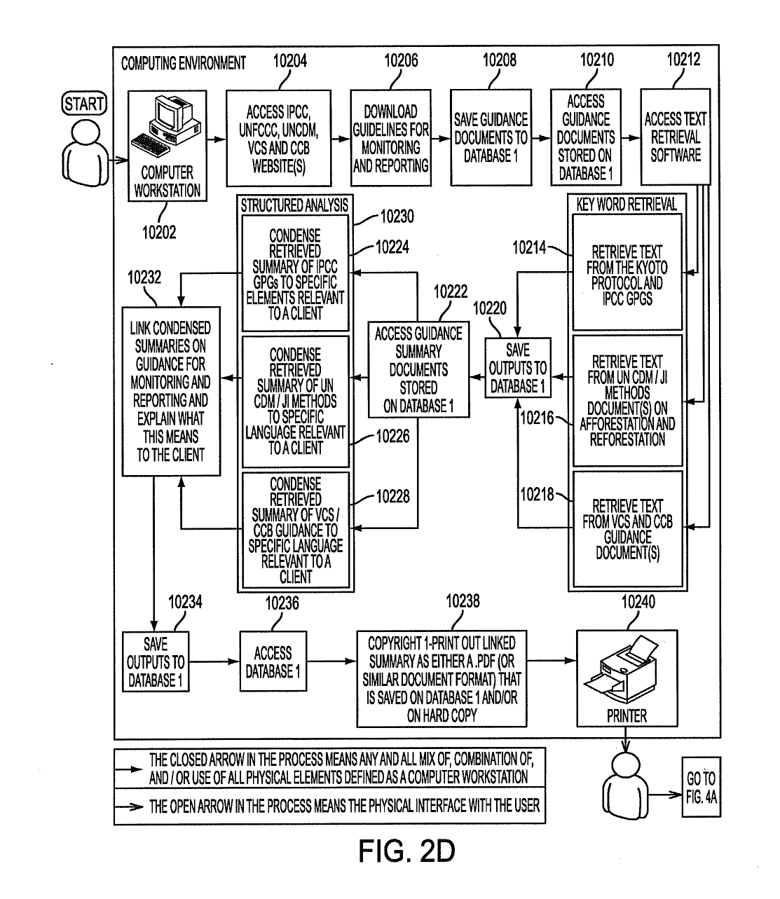

FIG. 2A shows the generic process used to develop a legal/policy analysis. A computer workstation 202 includes a screen display(s), processor(s), hard drive(s), a keyboard, a mouse, a router connected to the internet and other physical elements related to a computer workstation, etc. A computer workstation 202 in this context can store, retrieve, process and/or output data and/or communicate with other computers. The computer workstation is used to access the internet websites in 204 for the following: i) international multi-lateral and/or bi-lateral agreements, frameworks, protocols, and/or policy related the mitigation of climate change; ii) United Nations (UN), national and/or regional (i.e., sub-national and/or within a national territory) regulated trading schemes and/or standards; iii) voluntary trading schemes and/or standards. Material related to monitoring and reporting found through 204 is downloaded to the computer workstation in 206 and saved on Database 1 in 208. Once saved, the documents related to monitoring and/or reporting are accessed in 210 by the computer workstation. Text retrieval software is accessed in 212 by the computer workstation. The retrieval software can search for targeted key words in an electronic document, pull relevant text off the document and input the text to a new document. The reason text retrieval software is used is because many of the guidance documents for mitigating climate change are in excess of hundreds of pages and automated word search retrieval software is used to expedite the time to review the documents. The retrieved “summaries” and/or “reviews” for monitoring and/or reporting the vegetation attribute(s) in guidance documents are dealt with in for the following order: i) international multi-lateral and bi-lateral agreements on mitigation of climate change and/or greenhouse gases (GHGs) in 214; ii) international, national and regional regulated trading mechanisms related to climate change and/or GHGs in 216; and iii) in 218, voluntary trading mechanisms related to climate change and GHGs. Each of the three summaries/reviews on legal frameworks and trading mechanisms are saved and stored on Database 1 in 220 and accessed with the computer workstation in 222. A structured analysis is completed in 230 by linking each of the summary documents for guidance on climate change mitigation in 224, regulated trading mechanisms in 226 and voluntary trading mechanisms in 228. The output in 232 for the structured analysis is a summary linking all monitoring and reporting guidance in one document, which is then saved to Database 1 in 234. The outputs of 232 are accessed in 236, and defined as Copyright 1 in 238 that is printed out in either a Portable Document Format (i.e, .pdf and/or similar file format) digital file and/or in hard copy with a printer in 240.

FIG. 2B provides a more detailed description of the process used to retrieve text for key words from FIG. 2A. A user accesses a computer workstation in 12202 that includes screen display(s), processor(s), hard drive(s), a keyboard, a mouse, a router connected to the internet and other physical elements related to a computer workstation, etc. A computer workstation 12202 in this context can store, retrieve, process and/or output data and/or communicate with other computers. The computer workstation is used to access text retrieval software in 12204 and word processing software in 12208. In 12206, the guidance documents relevant to the client from steps 214 to 218 that are stored on Database 1 are accessed. The key word search is comprised of two levels for each of the three groupings of guidance documents. The Level 1 key word search in 12210 is for words/phrases and/or sentences that relate to, specify or define a target green house gas including emission, measurement and/or monitoring of the target greenhouse gas and/or an eco-region, in the context of a target policy being searched for compliance, for example: the vegetation attribute (i.e., “biomass”); “monitor”; “report”; “verification”; “carbon”; “definition”; “remote sensing”; “model,” etc. The key words are loaded into the text retrieval software by the user in 12212 and the search is completed for the documents defined as multilateral and bi-lateral agreements in 214. The outputs are a new file that is loaded in the word processing software. The user reviews the retrieved text in 12216 and identifies new key words for a Level 2 key word search in 12218 for documents defined in 214. Examples of Level 2 key words are the following: “dead wood”, “litter”, “soil”, “respiration”, “decomposition”, “production”, “stocks”, “land cover”, “forest land”; “grassland”; “tier”; “process”; “ecosystem”; “model”; “deforestation”; “degradation”; “devegetation”; etc. Mathematical symbols may also be retrieved in the Level 2 key word for key equations, such as: “Δ”; “+”; “−”; etc. The outputs of the Level 2 key word text retrieval are reviewed by the user in 12220. In 12222, the Level 1 and 2 key words are used by the user for key words to search and retrieve text in 12224 for the guidance documents for monitoring and/or reporting for the vegetation attribute defined from 216 as regulated trading mechanisms in 12226. The outputs from the Level 1 and Level 2 key word search for guidance documents on regulated trading mechanisms in 12228 are reviewed by the user and used identify a Level 3 key word search in 12230. The Level 3 key word search adds examples of the following: “IPCC”; “baseline”; “additionality”; etc. The text in the guidance documents for the regulated trading mechanisms is searched for the Level 3 key words and the outputs are reviewed in 12232. The key words for Levels 1, 2 and 3 are used in 12234 as inputs to the text retrieval software in 12236 for target guidance documents in 12238 defined from 218 as voluntary trading mechanisms. The text outputs from the retrieval for the voluntary documents are reviewed in 12240 and used by the user to identify Level 4 key words in 12242. Examples used as Level 4 key words are the following: “eligible”; “activity”; “AFOLU”; “project”; etc. The Level 4 key words are loaded in the text retrieval software and the search is completed for the guidance documents on the voluntary trading mechanism. The text outputs retrieved from the Level 4 key word search are reviewed by the user in 12244. Tables and figures are also retrieved in any of the search levels when the tables and figures are described by a key word. One or more outputs (for example, all) outputs from the text retrieval are stored on Database 1 in 12246. In 12248, key words from the key word search are stored on a meta-database in Database 1. According to an aspect of an embodiment, keywords are specified according to legal/policy information terminology to compile policy parameters for a target greenhouse gas of the legal/policy information.

FIG. 2C provides a more detailed description of the structured analysis from 230 in FIG. 2A. The reason for this hierarchical approach to structuring the compliance documents for monitoring and/or reporting the vegetation attribute is because the vegetation attribute will be monetized by the client as a commodity in mitigating climate change. The legal/policy synthesis must show how the guidance on monitoring is fungible across the legal/policy framework landscape that deals with monitoring and/or reporting. Alternatively, if the monitoring guidance, and thus the monitoring, is not fungible, such as a disconnect between international policy guidance on climate change mitigation and a voluntary mechanism, the client's accredited voluntary offset from the vegetation attribute may not be interchangeable under international treaties and/or regulated markets with a polluter's emission footprint. Furthermore, if the methods and science used to monitor the vegetation attribute do not comply with the monitoring and/or reporting guidance, in practice this would mean that the techniques used to quantify an amount of the vegetation attribute may not create a fungible offset valuation for the client to sell to a polluter through a trading mechanism. A computer workstation in 302 includes screen display(s), processor(s), hard drive(s), a keyboard, a mouse, a router connected to the internet and other physical elements related to a computer workstation, etc. A computer workstation 302 in this context can store, retrieve, process and/or output data and/or communicate with other computers. In 304, the computer workstation is used to access the summaries for guidance documents in steps 224-228 from FIG. 2A and word processing software in 306. The structured analysis uses compliance requirements in monitoring and reporting for international agreements, frameworks, protocols and/or policy on mitigation of climate change in 308. The user organizes the key word retrieval outputs from 12216 and 12220 into paragraphs. The text context in paragraphs are first organized by increasing page number for the same source guidance document and then organized by earliest to the most recent publication date for the source guidance document. The text content in 308 defines the science and math that is used to monitor the vegetation attribute. The content in 308 then explains how the science methods are organized and ranked into different tiers and/or approaches, and how higher ranked tiers and/or approaches supersede methods with lower rankings. Next, UN, national and/or regional regulated (i.e., legal mandated and/or required by the law) mechanisms are reviewed in 310. The user first organizes the key word retrieval outputs from 12228 and 12232 into paragraphs. The text content in the paragraphs are first organized by increasing page number for the same source guidance document and then organized by earliest to the most recent publication date for the source guidance document. The text content in 310 defines the science and math that is used to monitor the vegetation attribute for the regulated mechanism. 310 also includes discussion of any and/or all types of crediting activities under the regulated mechanism that require the monitoring of the vegetation attribute when the clients reports project activities to the mechanism. The content in 310 also lists one or more (for example, all) references to the source documents used in 308. Voluntary mechanisms are reviewed in 312. The user organizes the key word retrieval outputs from 12240 and 12244 into paragraphs. The text content in the paragraphs are first organized by increasing page number for the same source guidance document and then organized by earliest to the most recent publication date for the source guidance document. The text content in 312 defines the science, math and/or the specific text that states how monitoring the vegetation attribute should be completed for the voluntary mechanism. 312 also includes discussion of any and/or all types of crediting activities under the regulated mechanism that require the monitoring of the vegetation attribute when the clients reports project activities to the mechanism. The content in 312 lists all references to the source documents used in 308 and 310. The width of the circle in 308-312 also indicates the level of compliance application for the specified guidance document and degree in which the guidance document ranges in application to other guidance documents. For instance, compliance requirements in international agreements and policy on climate change mitigation are generally wider ranging than national and voluntary carbon markets, and thus it is more important to link compliance in both regulated and voluntary trading mechanisms back to international GHG agreements. Furthermore, regulated and voluntary guidance often cite international guidance on mitigation as the preferred method in monitoring and/or reporting. This is because international guidance, such as the IPCC's Good Practice Guidance, explains how one or more (for example, all) countries must report standardized and comparable annual emissions and removals as a signatory to the UNFCCC. Thus, project developers must comply with similar methods of monitoring and/or reporting. After each level in the hierarchy for guidance documents is organized from the text retrieval outputs, a bottom-up summary is written by the user at the end of each section for the regulated and/or voluntary mechanism(s). The bottom-up summary states how the lower tiered guidance document relates to the wider ranging guidance document. The term “relate” in this context means to establish a logical intellectual connection between two guidance documents and/or a statement about how one guidance document compares to another guidance document. In 314, the user states how the contents for the regulated emissions trading mechanism(s) from 310 relate to the contents from 308 for international multi-lateral and/or bi-lateral agreements, frameworks, protocols, etc. In 316, the user states how the contents for the voluntary trading mechanism(s) from 312 relate to the contents for the regulated trading mechanism(s) from 310. In 318, the user states how the contents for voluntary trading schemes from 312 relate to the contents for the international multi-lateral and bi-lateral agreements from 308. The outputs from steps 308, 310 and 314 are combined in the word processing software as the output for the summary on the regulated trading mechanism(s) relevant to the client in 320. The outputs from 308, 310, 312, 314, 316 and 318 are combined in the word processing software as the output the summary on the voluntary trading mechanism(s) relevant to the client in 322. National and/or regional monitoring, reporting and verification (MRV) for a regulated cap and trade systems require an independent assessment and comparison to meet the IPCC's definitions for verification. The output from 308 is used in 324 for a summary on guidance documents for MRV of national and/or regional cap and trade systems. The decision to include any and/or all of outputs 308-316 is at the discretion of the client and dependent upon the specific trading mechanism relevant to the client as aforementioned. The outputs from 320, 322, and/or 324 are saved to Database 1 in 326.

FIG. 2D shows an example for the generic process for a legal/policy analysis applied to guidance documents for the Voluntary Carbon Standard (VCS) and the Climate, Community and Biodiversity Alliance Standard (CCBA). The difference between FIGS. 2A and 2D is that FIG. 2D shows the actual implementation of the generic process FIG. 2A. A computer workstation 10202 includes a screen display(s), processor(s), hard drive (s), a keyboard, a mouse, a router connected to the internet and other physical elements related to a computer workstation, etc. A computer workstation 10202 in this context can store, retrieve, process and/or output data and/or communicate with other computers. The computer workstation is used to access the internet websites in 10204 for the following: i) Intergovernmental Panel on Climate Change (IPCC; <URL: http://www.ipcc.ch/>); ii) United Nations (UN) Clean Development Mechanism (CDM; <URL: http:/cdm.unfccc.int/index.html/>); iii) the Voluntary Carbon Standard (VCS; <URL: http://www.v-c-s.org/>) and Climate, Community and Biodiversity Alliance (CCBA; <URL: http://www.climate-standards.org/>). Material related to guidance on monitoring and reporting found through 10204 is downloaded to the computer workstation in 10206 and saved on Database 1 in 10208. Examples of the documents stored on the Database 1 are: i) The Kyoto Protocol; ii) The Revised 1996 IPCC Guidelines (IPCC, 1997); iii) IPCC “Good Practice Guidance for Land Use, Land-Use Change and Forestry” (GPG-LULUCF; IPCC, 2003a); iv) The IPCC “Definitions and Methodological Options to Inventory Emissions from Direct Human-induced Degradation of Forests and Devegetation of Other Vegetation Types” (IPCC, 2003b); v) 2006 IPCC Guidelines for National Greenhouse Gas Inventories” (IPCC, 2006); vi) UNFCCC Clean Development Mechanism (CDM) Methodologies for Afforestation & Reforestation; vii) The Voluntary Carbon Standard's “Guidance for Agriculture, Forestry and Other Land Use Projects” (VCS, 2008); viii) “The Climate, Community and Biodiversity Standards” (CCBA, Second Edition December, 2008; ix) national GHG reporting documentation such as the annual reports from the Australia National Carbon Accounting System (NCAS); etc. Once saved, the guidance documents related to monitoring and/or reporting are then accessed in 10210 by the computer workstation. Text retrieval software is accessed in 10212 by the computer workstation. The text retrieval software is used to search the guidance documents for key words and retrieve text off the guidance related to the key words. The text retrieval is used for the following: in 10214 for the Kyoto Protocol and the IPCC Good Practice Guidelines (GPGs) for Land Use Land Use Change and Forestry (LULUCF) and Agriculture, Forestry and Other Land Use (AFOLU); in 10216 the CDM methods for afforestation and reforestation (a/r); and in 10218 the VCS guidance on AFOLU and the CCBA standard guidance document. Each of the summaries/reviews from the text retrieval are saved and stored on Database 1 in 10220 and accessed with the computer workstation in 10222. A structured analysis is completed in 10230 by linking the Kyoto Protocol and the IPCC GPGs in 10224 and the CDM a/r methods in 10226 to the VCS guidance on AFOLU and CCBA standards guidance document (s) in 10228. The output in 10232 for the structured analysis is a summary that is assembled from all monitoring and reporting guidance, which is then saved to Database 1 in 10234. The outputs of 10232 are accessed in 10236, and defined as Copyright 1 in 10238 that is printed out in either a Portable Document Format (i.e, .pdf and/or similar file format) digital file and/or in hard copy with a printer in 10240.

The following is an example of a parameterized summary as well as written summary of guidance documents for monitoring and/or reporting for a project site that a client can use for submission to a governing body and/or a trading mechanism, for example, the Voluntary Carbon Standard and the Climate, Community and Biodiversity Alliance Standard and is defined as Copyright 1 in 10238 from FIG. 2D. The parameters in the summary are organized by the retrieved information from policy documents into multiple tiers. The parameters are definitions to be used to in monitoring a vegetation attribute. The parameters are used to tie the compliance guidance for monitoring target vegetation attribute to the methodology used to monitoring the target vegetation with remote sensing imagery that is developed in the science plan. The parameters are organized into different tiers of policy documents where each tier has a legal priority over another tier. The different tiers are linked according to legal priority. The linking is completed by pulling text for policy documents with less legal priority with key words from documents with greater legal priority. The text that is pulled from the documents with less legal priority explicitly states how the document with less legal priority is related to policy documents with greater legal priority. The linking can be user assisted and/or automated. In this example, 28 legal/policy information items are complied as parameters including metadata that describe the parameters and/or the legal/policy information and which can be stored and managed via a data structure (e.g., a database) representing review of the target legal/policy information.

Parameter 1. Target greenhouse gas to be monitored under an international multi-lateral policy agreement, for example, CO₂, CH₄, N₂O, HFCs, PFCs and SHF and the following key words: “emission”, “removals” “monitoring” and “reporting”. Parameter 1 is an example of one of the retrieved outputs in 10214 from FIG. 2D that are condensed in 10224 from FIG. 2D. For example, the Kyoto Protocol required Annex 1 parties to the Convention to reduce emissions of Green House Gases (GHGs, which Annex A defines as CO₂, CH₄, N₂O, HFCs, PFCs and SHF) to the percentage of 1990 emissions set out in Annex B to the Protocol. The Protocol assigned each Annex 1 party a maximum amount of emissions (“the assigned amount”) which it might emit during the first commitment period (2008 to 2012). The Protocol stated that parties might offset removals of GHGs that are a result from Land-Use Change and Forestry (“LULUCF”) against emissions from LULUCF sources. The Protocol mentioned that the monitoring and reporting of changes in carbon stocks for emissions and removals should be in accordance with the IPCC's Guidance on Good Practice for LULUCF in Decision 15 of the Conference of Parties (“COP/MOP”) 1 on the preparation of information required under Article 7 of the Protocol.

Parameter 2. Method(s) used to describe requirements to calculate monitoring of GHG activities derived from retrieved text from the earliest dated and accessible IPCC Guidance on Good Practice (GPG) document for LULUCF referenced in Parameter 1 for an international multi-lateral policy agreement. Thus, input from Parameter 1 is used to identify the document, if any, to use to develop Parameter 2. The text retrieval was derived from the chapter titles of the Revised 1996 IPCC Guidelines (IPCC, 1997) and the following key words: “biomass” and “increments”. Parameter 2 is an example of one of the retrieved outputs in 10214 from FIG. 2D that are condensed in 10224 from FIG. 2D. For example, the Revised 1996 IPCC Guidelines (IPCC, 1997) was the first document that instructed countries on how to establish monitoring activities in the following sectors: Energy, Industrial processes, Solvent and other product use, Agriculture, Land use change and forestry, and Waste. Equation 1 and Table 5.2 of the Reference Manual (IPCC, 1997, p. 5.19-5.20) specifically used the biomass growth increments to calculate annual above-ground biomass for reporting values. The Good Practice Guidance and Uncertainty Management in National Greenhouse Gas Inventories (IPCC, 2000) provided no supplementary guidance for monitoring the LULUCF sector developed in the Revised 1996 IPCC Guidelines.

Parameter 3. Method(s) used to describe requirements to calculate monitoring of GHG activities derived from retrieved text from the next earliest dated IPCC Guidance on Good Practice (GPG) document for LULUCF referenced in Parameter 1 for an international multi-lateral policy agreement. Thus, input from Parameter 1 is used to identify the document to develop Parameter 3. Parameter 3 is an example of one of the retrieved outputs in 10214 from FIG. 2D that are condensed in 10224 from FIG. 2D. The text retrieval as derived from text of “Good Practice Guidance for Land Use, Land-Use Change and Forestry” (GPG-LULUCF; IPCC 2003a) and the following key words: “biomass”, “increments”, “monitoring”, “land use”, “carbon”, “verification” and “remote sensing”. For example, the IPCC “Good Practice Guidance for Land Use, Land-Use Change and Forestry” (GPG-LULUCF; IPCC, 2003a), made two primary advances in defining and monitoring the LULUCF sector. First, GPG-LULUCF defined six land use categories necessary for monitoring the LULUCF sector. These categories were: 1) forested land, 2) cropland, 3) grassland, 4) wetland, 5) settled land and 6) other land. The second advance was that it defined five carbon pools necessary for monitoring each of the six land use categories. The five pools were: 1) above-ground biomass, 2) below-ground biomass, 3) dead wood, 4) litter and 5) soil organic matter. Chapter 5.7 of GPG-LULUCF discussed approaches to the verification of GHG inventories. Chapter 5.7 showed that remote sensing is applicable to monitoring the six land use categories and above-ground biomass. Chapter 5.7 also discussed ecosystem modeling approaches that were suitable for verification of the five carbon pools and referred to FOREST-BGC (Waring and Running, 1998) and Biome-BGC (Running and Coughlan, 1988; Running and Hunt, 1993; Running, 1994) as the only “well known examples” of ecosystem models that could be used in verification.

Parameters 4-11. Method(s) used to describe requirements to calculate monitoring of GHG activities derived from retrieved text from the recent dated IPCC Guidance on Good Practice (GPG) document for LULUCF referenced in Parameter 1 for an international multi-lateral policy agreement. Thus, input from Parameter 1 is used to identify the document to use to develop Parameter 4-11. Parameters 4-11 are examples of the retrieved outputs in 10214 from FIG. 2D that are condensed in 10224 from FIG. 2D. The text retrieval was derived from text of Volume 4 of the “2006 IPCC Guidelines for National Greenhouse Gas Inventories” (IPCC, 2006) and the following initial key words: “biomass”, “increments”, “monitoring”, “emission”, “removals”, “land use”, “carbon”, “verification” and “remote sensing”. Parameters 5-12 were then retrieved with the following key words: “dead wood”, “litter”, “soil”, “respiration”, “decomposition”, “production”, “stocks”, “land cover”, “forest land”; “grassland”; “tier”; “process”; “ecosystem”; “model”; “Δ”; “+”; “−”; “=” etc.

Parameter 4. Definitions for target greenhouse gas(s) in an eco-region. For example, the Volume 4 of the “2006 IPCC Guidelines for National Greenhouse Gas Inventories” (to be termed “GPG-2006”; IPCC, 2006) dealt with the Land Use, Land Use Change and Forestry (LULUCF), which was redefined by Volume 4 as the Agriculture, Forestry and Other Land Use (AFOLU) sector. GPG-2006 incorporated clear definitions of the carbon cycle processes in the scientific background. These definitions were (IPCC, 2006, Volume 4, Chapter 1, p. 1.6-1.8): “Gross Primary Production (GPP) is the uptake of CO₂ through photosynthesis;” “About half of the Gross Primary Production is respired by plants, and returned to the atmosphere, with the remainder constituting Net Primary Production (NPP), which is the total production of biomass and dead organic matter in a year;” “Net Primary Production minus losses from heterotrophic respiration (decomposition of organic matter in litter, dead wood and soils) is equal to the net carbon stock change in an ecosystem and, in the absence of disturbance losses, is referred to as Net Ecosystem Production (NEP);” “Net ecosystem production minus additional carbon losses from disturbance (e.g., fire), harvesting and land clearing during land-use change, is often referred to as Net Biome Production (NBP).” GPG-2006 stated that the “carbon stock change that is reported in national greenhouse gas inventories for land-use categories is equal to net biome production” (IPCC, 2006, Volume 4, Chapter 1, p. 1.7).

Parameter 5. Classification of uses of an area of land within an eco-region. For example, the generic equations to be used for annual monitoring and reporting of the Agriculture, Forestry and Other Land Use (AFOLU) sector under the IPCC Good Practice Guidance (IPCC, 2006, Volume 4, Chapter 2, Equations 2.1 and 2.3) are:

For example, Six land use classes:

ΔC_{AFOLU}=ΔC_{FL}+ΔC_{CL}+ΔC_{GL}+ΔC_{WL}+ΔC_{SL}+ΔC_{OL} Eq. 1

Where: ΔC is the carbon stock change amount; indices denote the following land-use categories: AFOLU is Agriculture, Forestry and Other Land Use; FL is Forest Land; CL is Cropland; GL is Grassland; WL is Wetlands; SL is Settlements; and OL is Other Land.

Parameter 6. Target greenhouse gas pool monitored in each class of area use within an eco-region. For example, Five carbon pools are monitored for each land use class:

ΔC_{LUi}=ΔC_{AB}+ΔC_{BB}+ΔC_{DW}+ΔC_{LI}+ΔC_{SO} Eq. 2

Where: ΔC_{LUi} is the carbon stock changes for a stratum of land-use category; subscripts denote the following carbon pools: AB is above-ground biomass; BB is below-ground biomass; DW is deadwood; LI is litter; and SO is soil organic matter. ΔC_{HWP} is the carbon stock change for Harvested Wood Products (HWP). HWP is included in the IPCC GPG 2006 for AFOLU, but it is dealt with separately in GPG-2006 when calculating the generic equations for carbon pools in Equation 2.

Parameter 7. Categories of area uses within an eco-region. For example, the IPCC Good Practice Guidance defined AFOLU land use categories required for reporting in the AFOLU sector as the following (IPCC, 2006, Volume 4, Chapter 3, p. 3.6-3.7):

Forested land—This category includes all land with woody vegetation consistent with thresholds used to define Forest Land in the national greenhouse gas inventory. It also includes systems with a vegetation structure that currently fall below, but in situ could potentially reach the threshold values used by a country to define the Forest Land category.

Cropland—This category includes cropped land, including rice fields, and agro-forestry systems where the vegetation structure falls below the thresholds used for the Forest Land category.

Grassland—This category includes rangelands and pasture land that are not considered Cropland. It also includes systems with woody vegetation and other non-grass vegetation such as herbs and brushes that fall below the threshold values used in the Forest Land category. The category also includes all grassland from wild lands to recreational areas as well as agricultural and silvi-pastural systems, consistent with national definitions.

Wetlands—This category includes areas of peat extraction and land that is covered or saturated by water for all or part of the year (e.g., peatlands) and that does not fall into the Forest Land, Cropland, Grassland or Settlements categories. It includes reservoirs as a managed sub-division and natural rivers and lakes as unmanaged sub-divisions.

Settlements—This category includes all developed land, including transportation infrastructure and human settlements of any size, unless they are already included under other categories. This should be consistent with national definitions.

Other Land—This category includes bare soil, rock, ice, and all land areas that do not fall into any of the other five categories. It allows the total of identified land areas to match the national area, where data are available. If data are available, countries are encouraged to classify unmanaged lands by the above land-use categories (e.g., into Unmanaged Forest Land, Unmanaged Grassland, and Unmanaged Wetlands). This will improve transparency and enhance the ability to track land-use conversions from specific types of unmanaged lands into the categories above.

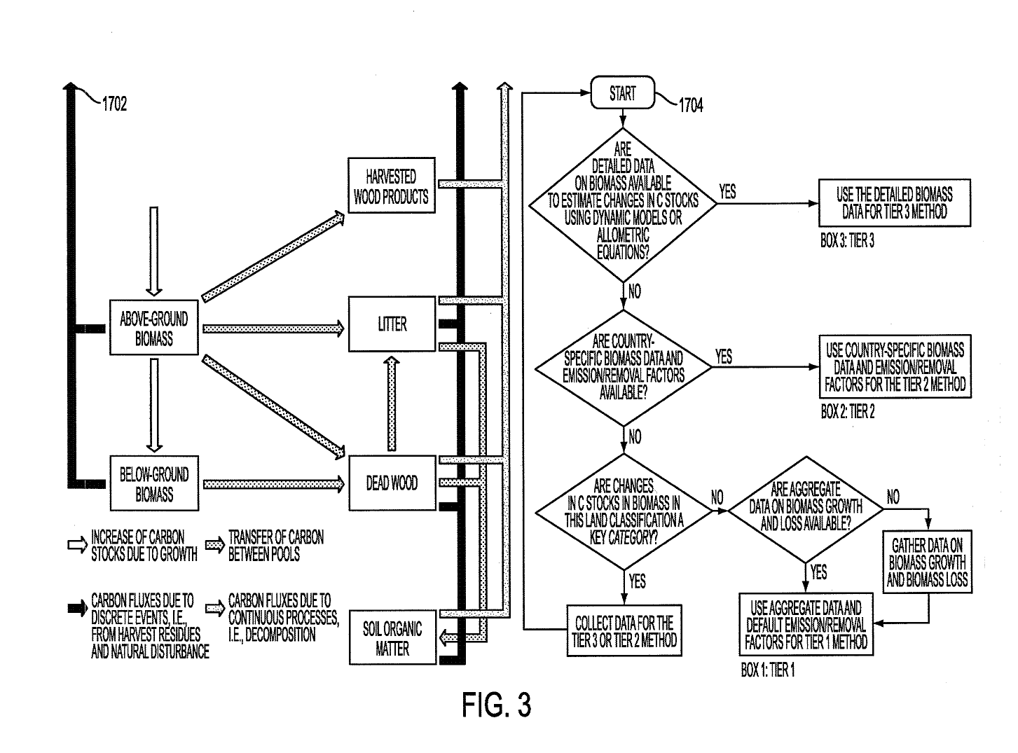

Parameter 8. Target greenhouse gas cycle definition. For example, FIG. 3 shows in 1702a generalized flowchart of the carbon cycle defined by the IPCC (retrieved from IPCC, 2006, Volume 4, Chapter 2, p. 2.8) and in 1704 (see FIG. 3) shows the generic decision tree for identification of appropriate tier to estimate changes in different carbon pools in each land use category (retrieved from IPCC, 2006, Volume 2, Chapter 2, p. 2.14). The generalized IPCC flowchart of the carbon cycle (in 1702, see FIG. 3) shows all five pools and associated annual fluxes including inputs to and outputs from the system, as well as all possible transfers between the pools (IPCC, 2006, Vol. 4, Ch 2, p. 2.8). Overall, carbon stock changes within each AFOLU land use stratum are estimated by adding up changes in all carbon pools by AFOLU Generic Equation 2.3 (IPCC, 2006, Volume 4, Chapter 2). The carbon cycle includes changes in carbon stocks due to both continuous processes (i.e., growth, decay) and discrete events (i.e., disturbances like harvest, fire, insect outbreaks, land-use change and other events). Continuous processes can affect carbon stocks in all areas in each year, while discrete events (i.e., disturbances) cause emissions and redistribute ecosystem carbon in specific areas (i.e., where the disturbance occurs) and in the year of the event.

Parameter 9. Definitions of target greenhouse gas pools. For example, the IPCC definition for each carbon pool is as follows (IPCC, 2006, Volume 4, Chapter 1, p. 1.9):

Biomass:

Above-ground biomass—All biomass of living vegetation, both woody and herbaceous, above the soil including stems, stumps, branches, bark, seeds and foliage.

Below-ground biomass—All biomass of live roots. Fine roots of less than 2 mm diameter are often excluded because these often cannot be distinguished empirically from soil organic matter or litter.

Dead Organic Matter (DOM): Dead Wood—includes all non-living woody biomass not contained in the litter, either standing, lying on the ground, or in the soil.

Dead wood includes wood lying on the surface, dead roots, and stumps, larger than or equal to 10 cm in diameter (or the diameter specified by the country).

Litter—Includes all non-living biomass with a size greater than the limit for soil organic matter and less than the minimum diameter chosen for dead wood, lying dead, in various states of decomposition above or within the mineral or organic soil. This includes the litter layer as usually defined in soil typologies. Live fine roots above the mineral or organic soil are included in litter where they cannot be distinguished from empirically.

Soils: Soil Organic Matter—Includes organic carbon in mineral soils to a specified depth chosen by the country and applied consistently through the time series. Live and dead fine roots and DOM within the soil, that are less than the minimum diameter limit (suggested 2 mm) for roots and DOM, are included with soil organic matter where they cannot be distinguished from it empirically.

Parameter 10. Methods used for estimating changes in a target greenhouse gas. For example, the IPCC GPG-2006 developed a decision tree for identification of appropriate tier to estimate changes in carbon stocks. 1704 (see FIG. 3) is the three-tiered system developed for decision-making. The decision tree begins by asking whether data on biomass is available to estimate changes in carbon stocks using dynamic models or Allometric Equations? If the answer is “yes” the decision tree indicates that the use dynamic models or Allometric Equations are the preferred methodology quantifying detailed biomass data, which is entitled a “Tier 3” approach. The Tier 2 approach is the use of country-specific biomass data and emissions/removal factors. The bottomed tiered approach, Tier 1, used aggregate data and default emission/removal factors for biomass found in the IPCC GPG-2006. The Tier 3 approach, therefore, supersedes Tier 1 and Tier 2 approaches if a Tier 3 approach is available. Further direction on development of Tier 3 approaches is found at the end of IPCC GPG-2006, Volume 4, Chapter 2, pages 2.50-2.53.

Parameter 11. For example, the IPCC 2006-GPG (Chapter 4, Section 4.2, p. 10-27) dealt with the estimation of forest carbon pools and described how this should be completed for each of the three tiered approaches for GHG reporting in AFOLU (see Section 4.4.5 of this document). GPG 2006 (Vol. 4, Ch. 4, p. 4.34, Box 4.3) identified The Australia National Carbon Accounting System (NCAS) as an example of a good practice approach in AFOLU sector monitoring.

Method(s) used to describe requirements to calculate monitoring of GHG activities derived from retrieved text from the The Australia National Carbon Accounting System (NCAS) in the year 2006 (<URL: http://www.climatechange.gov.au>) for LULUCF referenced in Parameter 11 from GPG-2006. Thus, information from Parameter 12 is used to identify the document to use to develop Parameter 12. Parameter 12 is an example of one of the retrieved outputs in 10214 from FIG. 2D that are condensed in 10224 from FIG. 2D. Since NCAS is also an example of a national monitoring system, a more in-depth review of parameters from NCAS could be developed as an example of a national monitoring system in 216 and of FIG. 2A. The following key words were used in the retrieval search: “biomass”, “increments”, “remote sensing”, “carbon”, and “model.

Parameter 12. For example, the NCAS LULUCF/AFOLU sector model, CamFor integrated the Roth-C soil carbon model (Jenkinson et. al., 1987, Jenkinson et. al., 1991), the 3-PG forest growth model (Landsberg and Waring 1997) and the GENDEC litter decomposition model (Moorhead and Reynolds 1991; Moorhead et. al., 1999). 3-PG forest growth model was used with NOAA-AVHRR remote sensing data and climate data to model NPP.

At this point, a paragraph is constructed and/or assembled in the word processing software explaining what the retrieved text means in simple language interpretable to anyone skilled in the art. This is an example of a linked summary the documents in 10232 for documents in 10224 from FIG. 2D. For example, this means that Net Biome Production (NBP) is the sum of all five carbon pools within all six land use categories (IPCC, 2006, Volume 4, Chapter 2, Equations 2.1 and 2.3). Thus, remote sensing-derived measurements of NPP can be used to monitor biomass and incorporated with ecosystem modeling procedures, such as with the process-based dynamic ecosystem model Biome-BGC, to monitor, report and/or verify all five carbon pools defined as Net Biome Production. Remote sensing of Gross Primary Production and Net Primary Production automatically accounts HWP removals lost throughout a year because the remote sensing images of vegetation growth are at increments of every 8 to 10 days. In other words, remote sensing-derived GPP and NPP is vegetation growth after disturbance and Biome-BGC derived GPP and NPP is vegetation growth before disturbance. Furthermore, IPCC GPG-2006 separates HWP from NBP and also deals with reporting HWP separately. Heterotrophic respiration is the annual carbon flux amount loss to the atmosphere due to decomposition. The IPCC GPGs (2006) define Net Ecosystem Production (NEP) as the numerical difference between heterotrophic respiration and Net Primary Production (NPP). Heterotrophic respiration is not formally accounted for in GPG-2006 other than the reference to NEP and the use of average decomposition rates in relevant carbon pools (i.e., litter and soil out flux). CamFor is a Tier 3 approach for national GHG reporting in AFOLU. The CamFor model complied with the IPCC Revised 1996 Guidelines for National Greenhouse Gas Inventories (IPCC, 1997) and the IPCC Good Practice Guidance for Land Use, Land Use Change and Forestry (IPCCa, 2003) while taking into account Australian conditions. The CamFor model also benefited from Australia's uniqueness of having Landsat and NOAA AVHRR satellite sensor receiving stations. This allowed NCAS to develop a long-term Landsat and NOAAAVHRR imagery archive. These long-term imagery archives for Australia make application of the full CamFor model with Landsat and NOAAAVHRR imagery nearly impossible for countries that do not have such recieving stations. Finally, the NCAS methods have not been updated to comply with the IPCC GPG-2006 for AFOLU, as is stated on the NCAS website.

Parameters 13-16. Degradation specification or identification for/within the target eco-region for monitoring GHG activities within the target eco-region. For example, method(s) used to describe requirements to calculate monitoring of GHG activities derived from retrieved text from the recent dated IPCC Guidance on Good Practice (GPG) document for LULUCF referenced in Parameter 1 for an international multi-lateral policy agreement. Thus, input from Parameter 1 is used to identify the document to use to develop Parameters 13-16. Parameters 13-16 are an example of the retrieved outputs in 10214 from FIG. 2D that are condensed in 10224 from FIG. 2D. The text retrieval was derived from text of the IPCC “Definitions and Methodological Options to Inventory Emissions from Direct Human-induced Degradation of Forests and Devegetation of Other Vegetation Types” (IPCC, 2003b). This document is not in order by publication date with the other documents due to the specific nature of the document. This document was consistent with the monitoring procedures set forth by GPG-LULUCF (IPCC, 2003a), which was later superseded by GPG-2006. The following initial key words: “biomass”, “monitoring”, “emission”, “removals”, “land use”, “carbon”, “verification”, “remote sensing”, “definition”, “dead wood”, “litter”, “soil”, “respiration”, “decomposition”, “production”, “stocks”, “land cover”, “forest land”; “grassland”; “tier”; “process”; “ecosystem”; “model”; “Δ”; “+”; “−”; “=” etc.

Parameter 13. General definition of degradation. For example, the IPCC “Definitions and Methodological Options to Inventory Emissions from Direct Human-induced Degradation of Forests and Devegetation of Other Vegetation Types” (IPCC, 2003b); to be referred to hereafter as the “IPCC Degradation Report”). The IPCC Degradation Report specifically defined the term “degradation” to be associated with forested land and “devegetation” specifically associated with cropland, grassland and wetland land use categories.

Parameter 14. Definition of forest degradation. For example, the IPCC Degradation Report reviewed 50 definitions for forest degradation, but “none of [them was] found to be suitable for operational use in the context of the Kyoto Protocol (IPCC, 2003b, p. 11)”. The IPCC Degradation Report defined forest degradation as: “A direct human-induced long-term loss (persisting for X years or more) of at least Y % of forest carbon stocks [and forest values]since time Tan d not qualifying as deforestation or an elected activity under Article 3.4 of the Kyoto Protocol (IPCC, 2003b, p. 16).”

Parameter 15. Definition of forest degradation. For example, the IPCC Degradation Report mentioned that the land use area, time and carbon loss thresholds were “unspecified” because of operational differences between countries. In terms of monitoring forest degradation with remote sensing, the IPCC degradation report stated that “remote sensing remains one of the most efficient means of detecting activities across broad spatial extents that impact forests” (IPCC, 2003b, p. 19). The IPCC Degradation Report linked forest degradation to the carbon pools discussed in GPG-LULUCF (IPCC, 2003a), and specifically stated that “defining forest degradation based on changes in biomass may be the most straightforward to implement and can be directly related to estimates of all relevant forest carbon pools (IPCC, 2003b, p. 16).”

Parameter 16. Definition of devegetation. For example, the IPCC Degradation Report stated that they found “very few published definitions of devegetation and they are essentially the corollaries of deforestation” (IPCC, 2003b, p. 17) in other land use types. The IPCC Degradation Report stated, “The Marrakesh Accords do not define devegetation. The authors state that the Accords do define revegetation as ‘a direct human-induced activity to increase carbon stocks on sites through the establishment of vegetation that covers a minimum of 0.05 hectares and does not meet the definitions of afforestation and reforestation . . . ’ (IPCC, 2003b, p. 17).” The IPCC Degradation Report stated that the following definition met the operational necessities for monitoring devegetation of other vegetation types in the context of the Kyoto Protocol:

“A direct human-induced long-term loss (persisting for X years or more) of at least Y % of vegetation [characterized by cover/volume/carbon stocks]since time Ton vegetation types other than forest and not subject to an elected activity under Article 3.4 of the Kyoto Protocol. Vegetation types consist of a minimum area of land of Zhectares with foliar cover of W % (IPCC, 2003b, p. 19).”

At this point, a paragraph is constructed and/or assembled in the word processing software explaining what the retrieved text means in simple language interpretable to anyone skilled in the art. This is an example of a linked summary the documents in 10232 for documents in 10224 from FIG. 2D. For forest degradation, this means that remote sensing-derived measurements of standing biomass stored in wood and vegetation growth values of Net Primary Production and Net Biome Production are applicable to the monitoring of forest degradation of biomass in the context of The Degradation Report and GPG-2006. With regard to monitoring devegetation of other vegetation types, The IPCC Degradation Report implied that remote sensing had the same monitoring capabilities as with forest degradation. This means that remote sensing-derived measurements for vegetation growth of Net Primary Production and Net Biome Production can be used to monitor biomass devegetation of other vegetation types in the context of non-forested land under the IPCC Degradation Report and GPG-2006.

Parameters 17-23. Voluntary greenhouse gas trading mechanisms. Method(s) used to describe requirements to calculate monitoring of GHG activities derived from retrieved text from the Voluntary Carbon Standard's “Guidance for Agriculture, Forestry and Other Land Use Projects” (VCS, 2008) document is used as an example of guidance from a voluntary mechanism. Parameters 17-23 are an example of the retrieved outputs in 10218 from FIG. 2D that are condensed in 10228 from FIG. 2D. The VCS document is relevant to parameters 17-23, except for parameter 19. Parameter 19 is for the UN CDM Approved Consolidated Methodologies for Afforestation & Reforestation and was informed of for use by the Voluntary Carbon Standard guidance. Parameter 19 is an example of the retrieved outputs in 10216 from FIG. 2D that are condensed in 10226 from FIG. 2D. The CDM Guidance is an example of a regulated mechanism. The reason this is a brief review of the CDM methods is because the intent of this section is an example of the VCS guidance, which in turn references the CDM methods. The text retrieval for both VCS and CDM documents used the following as examples of key words: “IPCC”, “GPG”, “biomass”, “monitoring”, “land use”, “carbon”, “verification”, “remote sensing”, “definition”, “dead wood”, “litter”, “soil”, “respiration”, “decomposition”, “production”, “stocks”, “land cover”, “forest land”; “grassland”; “tier”; “process”; “ecosystem”; “model”; “deforestation, “degradation” and the following symbols “Δ”; “+”; “−”; “=” etc.

Parameter 17. General definition for monitoring requirements of offset activities under the VCS mechanism. For example, the Voluntary Carbon Standard's “Guidance for Agriculture, Forestry and Other Land Use Projects” (VCS, 2008; and to be referred to hereafter as “The VCS AFOLU Document”) provided guidance for Voluntary Carbon Units (VCUs) in the AFOLU sector. The general VCS guidance on estimating GHG removals stated on page 28: “VCS AFOLU methodologies provide guidance for estimating net GHG benefits from project activities against the baseline scenario following the methodologies outlined in the IPCC Guidelines 2006 for AFOLU.” The VCS Document also stated the following for monitoring net emissions reductions and GHG removals for all AFOLU projects: “To be eligible under the VCS, AFOLU projects must have robust and credible monitoring protocols as defined in the approved methodologies. Monitoring and ex-post quantification of the project scenario (including off-site climate impacts) must follow the applicable guidance available in approved A/R CDM methodologies and/or IPCC documents (VCS, 2008, p. 31).”

The VCS AFOLU project activities were grouped into four categories. The following subsections are a brief review of standards required for monitoring and verification of the four VCS AFOLU categories.

Parameter 18. Definition of vegetation attribute monitoring requirements for an offset project activity related to Afforestation, Reforestation and Revegetation (ARR) under the VCS mechanism. For example, the VCS AFOLU Document stated that “eligible activities in the Afforestation, Reforestation and Revegetation (ARR) project category consist of establishing, increasing or restoring vegetative cover through the planting, sowing or human-assisted natural regeneration of woody vegetation to increase carbon stocks in woody biomass and, in certain cases, soils. Examples of envisaged VCS ARR activities included: reforestation of forest reserves; reforestation or revegetation of protected areas and other high priority sites; reforestation or revegetation of degraded lands; and rotation forestry with long harvesting cycles (VCS, 2008, p. 9).” Eligible carbon pools for VCS credits are above-ground biomass, below-ground biomass, dead wood, litter, soil organic matter and harvested wood products. Page 29 of The VCS AFOLU Document directed carbon pool monitoring for ARR to follow “the guidance provided by the IPCC or approved Afforestation and Reforestation (A/R) CDM methodologies.” Furthermore, on page 6, The VCS AFOLU Document stated that Validators & Verifiers are considered “accredited” [for all four VCS ALOLU sectors] under the VCS if they are accredited for scope 14 (Afforestation & Reforestation) of the UNFCCC Clean Development Mechanism (CDM). Two meanings for compliance are interpreted from this statement: 1) that VCS ARR is linked to CDM compliance on carbon pool monitoring and verification for Afforestation & Reforestation and 2) rules established for CDM compliance on carbon pool monitoring and verification supersede those of the VCS, because accreditation as a project verifier under CDM methods preempts a verifier gaining accreditation under the VCS.

Parameter 19. Definition of vegetation attribute monitoring requirements for an offset project activity related to Afforestation and Reforestation under the CDM mechanism. For example, the Approved Consolidated Methodologies for Afforestation & Reforestation” provided guidance for carbon pool monitoring and verification under the CDM. On page 3, the document states that above-ground and below-ground biomass are required for monitoring and verification. Dead wood, litter and soil organic matter are required if the data is available, or alternatively, are not required if the data is not available.

Parameter 20. Definition of vegetation attribute monitoring requirements for an offset project activity related to Agricultural Land Management (ALM) under the VCS mechanism. For example, the VCS AFOLU Document stated that “land use and management activities that have been demonstrated to reduce net greenhouse gas (GHG) emissions on cropland and grassland (see IPCC 2006 GL for AFOLU) by increasing carbon (C) stocks (in soils and woody biomass) and/or decreasing CO₂, N₂O and/or CH₄ emissions from soils are eligible for certification under the VCS as Agricultural Land Management (ALM) projects (VCS, 2008, p. 10).” The VCS AFOLU Document mentioned three categories for ALM activities are: (A) improved cropland management; (B) improved grassland management (C) cropland and grassland land-use conversions. The VCS AFOLU Document also stated on page 18 that “the primary carbon pool of concern for ALM is soil carbon. Since the definition of ALM included reference to reporting GHG emissions from IPCC GPG-2006, it is straightforward that monitoring should be in line with the IPCC GPG-2006 for AFOLU. Page 30 of The VCS Document linked monitoring of ALM to the IPCC GPG-2006 in AFOLU and reviewed the 3 Tiered approach found in GPG-2006 (see Section 4.4.5 of this document).

Parameter 21. Definition of vegetation attribute monitoring requirements for an offset project activity related to Improved Forest Management (IFM) under the VCS mechanism. For example, VCS AFOLU Document stated that Improved Forest Management (IMF) activities are implemented on forest lands managed for wood products. These areas are designated, sanctioned or approved for such activities (e.g., such as logging concessions or plantations) by the national or local regulatory bodies and are eligible for crediting under the VCS IFM category. IFM activities are intended to increase carbon stocks and reduce GHGs. IFM activities included reduced impact logging, conversion of logged forests to protected areas, extending the rotation age of evenly managed forests and conversion of low-productive forests to high productive forests. All carbon pools are required for monitoring and verification of IFM under the VCS (VCS, 2008, p. 18). The VCS AFOLU Document also stated on page 30 that: “To date, no approved methodologies exist for IFM project activities under the UNFCCC. Guidance for estimating carbon stocks and changes in them is provided in the IPCC GPG-2006 (Chapter 4, Section 4.2, p. 10-27).”

Parameter 22. Definition of vegetation attribute monitoring requirements for an offset project activity related to Reduced Emissions for Deforestation and forest Degradation (REDD) under the VCS mechanism. For example, the VCS AFOLU Document stated that: “activities that reduce the conversion of native or natural forests to non-forest land, which are often coupled with activities that reduce forest degradation and enhance carbon stocks of degraded and/or secondary forests that would be deforested in absence of the Reduced Emission from Deforestation and Forest Degradation (REDD) project activity, are creditable as REDD section under the VCS (VCS, 2008, p. 13).” Deforestation can be planned (designated and sanctioned) or unplanned (unsanctioned) activities within a country. The VCS AFOLU Document defined planned deforestation activities as the following: “national resettlement programs from non-forested to forested regions; national land plans to reduce the forest estate and convert it to industrial-scale production of commodities such as soybeans, pulpwood, and oil palm; plans to convert well-managed community-owned forests to other non-forest uses; or planned forest conversion for urban, rural, and infrastructure development (VCS, 2008, p. 13).” Unplanned deforestation activities are defined as: “activities that occur as a result of socio-economic forces that promote alternative uses of forested land, and the inability of institutions to control these activities; such as population growth and the expansion of roads and other infrastructure leading to subsistence food production and fuelwood gathering taking place on lands not designated for such activities (VCS, 2008, p. 13).” The VCS AFOLU Document stated that the following REDD practices are eligible activities under the VCS (VCS, 2008, p. 14):

1) Avoiding planned deforestation (APD): Reduces GHG emissions by stopping deforestation on forest lands that are legally authorized and documented to be converted to non-forest land.

2) Avoiding unplanned frontier deforestation and degradation (AUFDD): Reduces GHG emissions by stopping deforestation/degradation of degraded to mature forests at the forest frontier that has been expanding historically, or will expand in the future, as a result of improved forest access, often through construction of roads.

3) Avoiding unplanned mosaic deforestation and degradation (AUMDD): Reduces GHG emissions by stopping deforestation/degradation of degraded to mature forests occurring in a mosaic pattern.

Parameter 23. Definition of monitoring requirements for Reduced Emissions for Deforestation and forest Degradation (REDD) under the VCS mechanism. For example, the VCS Document stated on page 19 that all carbon pools are required for REDD monitoring activities. The VCS Document directly linked VCS REDD monitoring and reporting to the IPCC Good Practice Guidance for AFOLU, and stated the following (VCS, 2008, p. 31): “the IPCC 2006 Guidelines provide[d] guidance for estimating forest regrowth (carbon accumulation) if degradation is reduced, and for estimating reductions in forest carbon stocks caused by removals of biomass exceeding regrowth. Monitoring and estimation methods currently must be based on the IPCC Guidelines.”

At this point, a paragraph is constructed and/or assembled in the word processing software explaining what the retrieved text means in simple language interpretable to anyone skilled in the art. This is an example of a linked summary the documents in 10232 for documents in 10228 from FIG. 2D when the review is for the VCS mechanism. This is an example of a linked summary the documents in 10232 for documents in 10226 from FIG. 2D when the review is for the CDM mechanism. The basic point of the retrieved information is that all VCS AFOLU projects must comply with the monitoring methodologies for GHG reporting in the IPCC Good Practice Guidance and UNFCCC CDM methodologies. Furthermore, the retrieved text means the following for the four VCS categories: 1) ARR projects must comply with the monitoring methodologies for GHG reporting in the IPCC Good Practice Guidance, UNFCCC CDM methodologies and can draw upon methods from peer-reviewed literature; 2) ALM projects must comply with the monitoring methodologies for GHG reporting in the IPCC Good Practice Guidance and can draw upon methods from peer-reviewed literature; 3) IFM projects must comply with the monitoring methodologies for GHG reporting in the IPCC GPG-2006, IPCC GPG-LULUCF and can draw upon methods from peer-reviewed literature and 4) REDD projects must comply with the monitoring methodologies for GHG reporting in the IPCC Good Practice Guidance and can draw upon methods from peer-reviewed literature. For CDM methods relevant to VCS ARR, all methodological equations stem from the IPCC “Good Practice Guidance for Land Use, Land-Use Change and Forestry” (GPG-LULUCF; IPCC, 2003a, see section 4.3 of this document) and peer-reviewed literature. This means that CDM methods for monitoring and verification follow the IPCC Good Practice Guidance.

Parameters 24-28. Method(s) used to describe requirements to calculate monitoring of GHG activities derived from retrieved text from the “The Climate, Community and Biodiversity Standards” (CCBA, Second Edition December, 2008) document as an example of guidance of a voluntary mechanism. The CCBA document is relevant to parameters 24-28. Parameters 24-28 are an example of the retrieved outputs in 10218 from FIG. 2D that are condensed in 10228 from FIG. 2D The text retrieval for the CCBA Standard documents used the following as examples of key words: “IPCC”, “GPG”, “biomass”, “monitoring”, “land use”, “carbon”, “verification”, “remote sensing”, “definition”, “dead wood”, “litter”, “soil”, “respiration”, “decomposition”, “production”, “stocks”, “land cover”, “forest land”; “grassland”; “tier”; “process”; “ecosystem”; “model”; “deforestation, “degradation” and the following symbols “Δ”; “+”; “−”; “=” etc. In the example provided, the retrieval only focuses on the climate component for compliance with The CCBA Standards related to carbon monitoring and verification for CCBA accredited land-based carbon projects.

Parameter 24. General definition for monitoring requirements of offset activities under the CCBA mechanism. For example, the CCBA Standards are intended for any land-based project including Reduce Emissions through avoided Deforeststion and forest Degradation (REDD) as well as those that remove carbon dioxide through sequestration.

Parameter 25. Definition for monitoring requirements of the original site condition under the CCBA mechanism. For example, Section G.1 on describing the original project site conditions: when describing the original site conditions for climate, The CCBA Standards stated that: “current carbon stocks [must be accounted for] within the project area(s), using stratification by land-use or vegetation type and methods of carbon calculation (such as biomass plots, formulae, default values) from the IPCC's 2006 Guidelines for AFOLU or a more robust and detailed methodology.”

Parameter 26. Definition for monitoring requirements of the baseline projections under the CCBA mechanism. For example, Section G.2 on baseline projections: the project baseline projections are intended to provide a “without” project reference scenario. Or in other words, what would happen at the project site if the CCBA accredited project did not occur. Points 1 and 3 related to GHG monitoring and are linked the IPCC Good Practice Guidance. Point 1 stated that the “land-use scenario in the absence of the project” should be described “following IPCC 2006 GL for AFOLU or a more robust and detailed methodology (CCBA, 2008, p. 14).” Point 3 stated that carbon stock changes should be calculated for the absence of project scenario. Estimation of carbon stocks is required for all land-use classes and carbon pools required in the IPCC GPG 2006 for AFOLU (CCBA, 2008, p. 14; and see section 4.4.1 of this document for further detail on these GHG reporting requirements). Project accounting is required for the timeframe of either the project lifetime or accounting period. GHG emissions from CH₄ and N₂O are also required for reporting the without project reference scenario.

Parameter 27. Definition for monitoring requirements of project impacts under the CCBA mechanism. For example, Section CL.1 on net positive climate impacts: CCBA accredited projects must generate a positive impact on atmospheric GHG concentrations from land use change within the project site and during the project's lifetime. “Net changes in carbon stocks due to project activities must be estimated using the methods of calculation, formulae and default values of the IPCC Guidelines for AFOLU or a more robust and detailed methodology (CCBA, 2008, p. 22).” Emissions of CH₄ and N₂O must be estimated “with” the project activity. GHG emissions resulting from the following project activities must also be quantified: biomass burning, fossil fuel combustion, synthetic fertilizers and decomposition of nitrogen fixing species.

Parameter 28. Definition for monitoring requirements of project impacts under the CCBA mechanism. For example, Section CL3 on climate impact monitoring: before a project is initiated, the project developers must have a monitoring plan in place to quantify and document changes (within and around the project boundaries) in project-related carbon pools, GHG emissions, and non-CO₂ GHG emissions if appropriate (CCBA, 2008, p. 24). Potential carbon pools to be included are: above-ground biomass, below-ground biomass, litter, dead wood, harvested wood products, soil carbon and peat. Carbon pools expected to decrease “must” be monitored. A full monitoring plan must be developed within six months of the project start date.

At this point, a paragraph is constructed and/or assembled in the word processing software explaining what the retrieved text means in simple language interpretable to anyone skilled in the art. This is an example of a linked summary the documents in 10232 for documents in 10228 from FIG. 2D when the review is for the CCBA mechanism. For example, this means that The CCBA Standards required methods that described the original conditions of a project site and monitoring methodologies during project implementation period to comply with the IPCC Good Practice Guidance and can draw upon methods from peer-reviewed literature.

A science plan is developed from the outputs of the legal/policy analysis. The reason a science plan is developed is to provide an intellectual bridge between what is required by the trading mechanism and the methods and techniques that are used to monitor the vegetation attribute at a project site for a client. With no legal/policy analysis, the science has no intellectual link to the requirements for monitoring and/or reporting of a vegetation attribute for the trading mechanism(s) relevant to the client. The legal/policy analysis shows that two types of vegetation attribute information are required by the client to comply with annual monitoring and/or reporting. The two types of vegetation attribute information are 1) a numerical biophysical element and 2) a land classification element indicating a specific land use. The primary spatial and temporal information used to monitor a vegetation attribute at a client's project site is with remote sensing imagery. Remote sensing imagery means digital images from satellite-borne active and/or passive sensors with measurement application to monitoring vegetation. Examples of remote sensing imagery from active sensors include Light Detection and Ranging (LiDAR) sensors and Synthetic-aperture radar (SAR) sensors. Passive sensors collect reflectance of electromagnetic radiation from the earth's surface. Remote sensing imagery may also be obtained from an unmanned aerial vehicle (i.e., an unmanned aircraft system). There are many satellites in orbit around the earth that can be used to develop a science plan and a database, so that a client's vegetation attribute can be monitored. The key initial component of the science plan is a strategic assessment of the capabilities of sensing instruments onboard current and future planned satellite missions, so that a sensor can be chosen that best implements the desired monitoring and/or reporting required by a client.

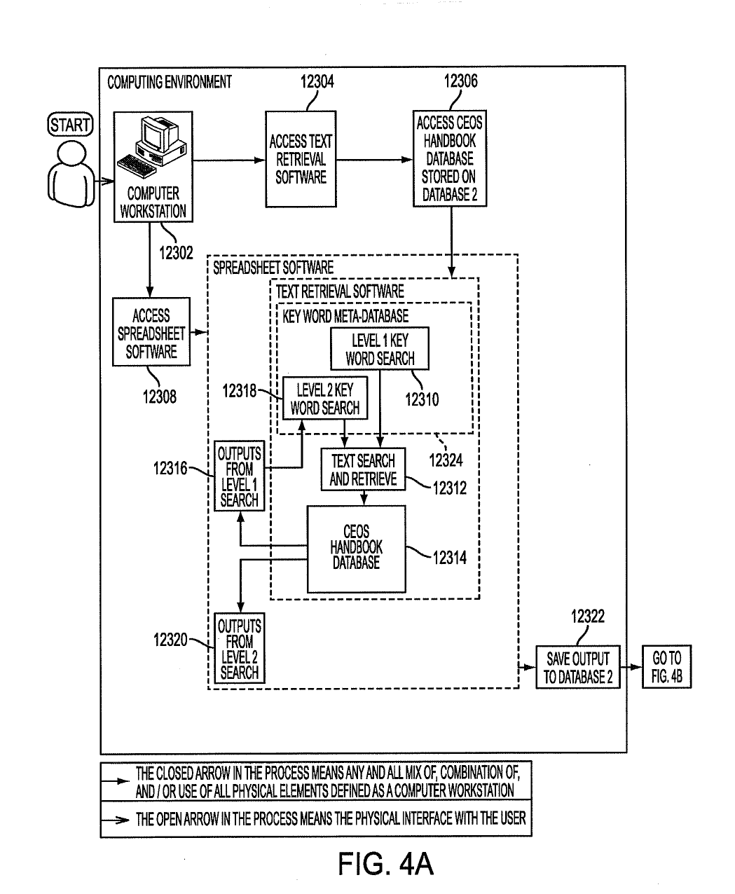

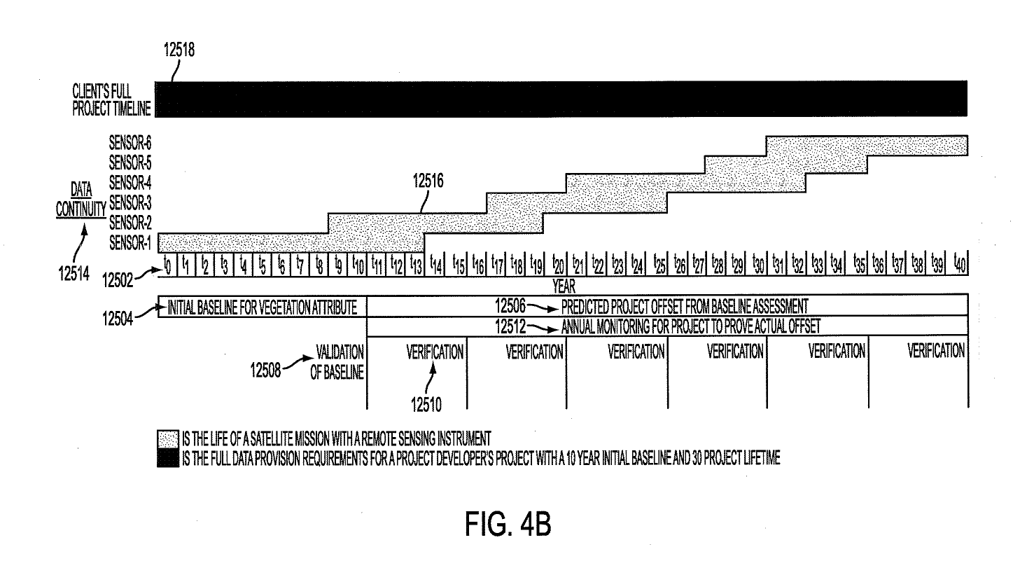

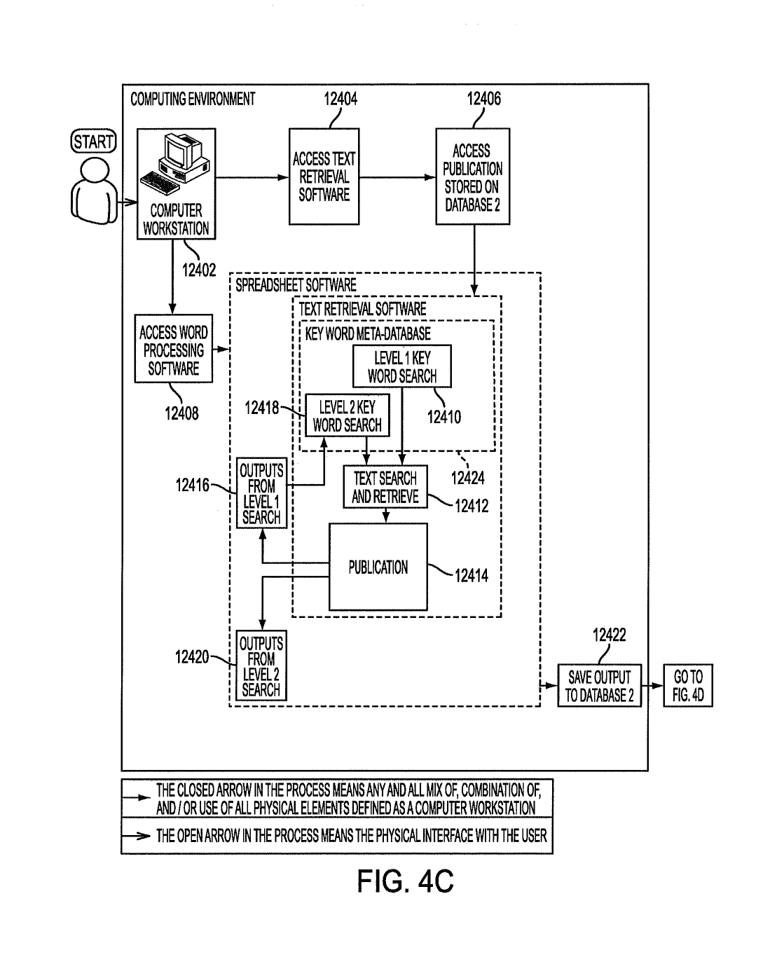

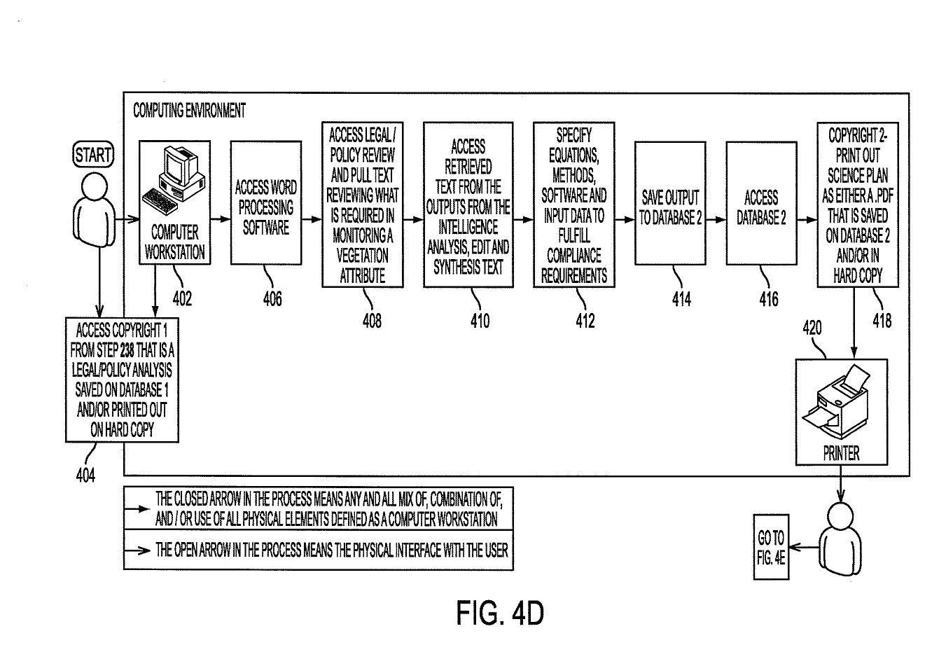

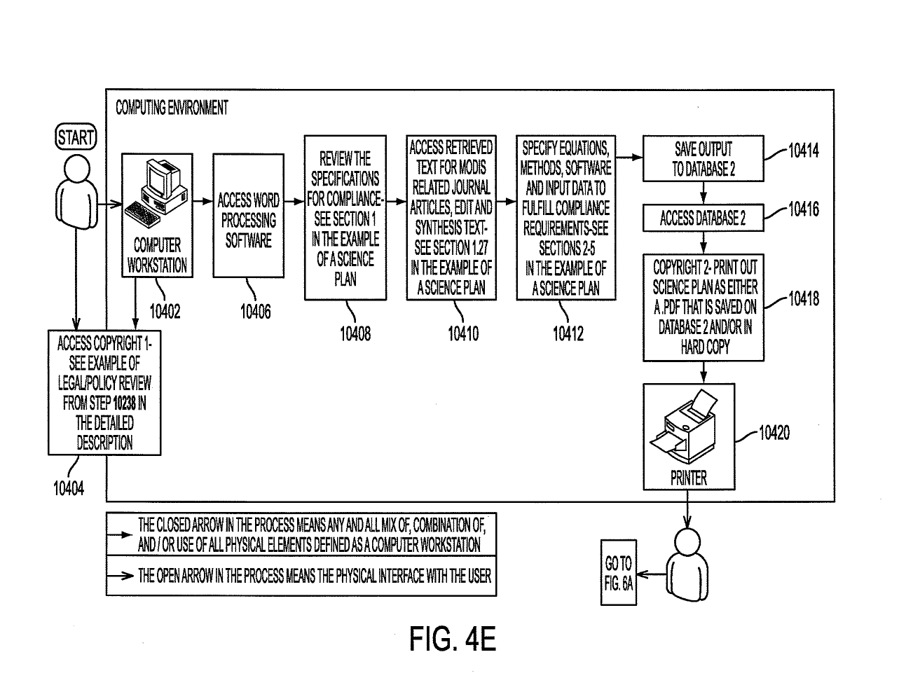

FIG. 4A shows the process used to retrieve text from the database for current and planned satellite missions. A database on current and future planned satellite missions and instrument sensors is developed on Database 2. The Committee on Earth Observation Satellites (CEOS) Handbook provides a regularly updated database of nearly all current and future planned satellite missions and sensing instruments <URL: http://www.eohandbook.com/>. The CEOS Handbook databases are downloaded and stored on Database 2. The information available on the satellite instruments (i.e., the sensor) includes: the satellite mission; the status of the mission; the type of sensor imagery; measurement applications; resolution; swath; accuracy; and other technical characteristics of the instrument. Text retrieval software is used to retrieve information from the CEOS databases and the process is exemplified in FIG. 4A. A user accesses a computer workstation in 12302 that includes screen display(s), processor(s), hard drive(s), a keyboard, a mouse, a router connected to the internet and other physical elements related to a computer workstation, etc. A computer workstation 12302 in this context can store, retrieve, process and/or output data and/or communicate with other computers. The computer workstation is used to access text retrieval software in 12304 and spreadsheet software in 12308. In 12306, the text retrieval software is used to access the CEOS Handbook database. The Level 1 key word search in 12310 for the CEOS Handbook database uses any and/or all the following key words as examples: “vegetation”, “global”, “forest”, “crop”, “land”, etc. The key words are entered into the text retrieval software in 12312, which is processed with the CEOS Handbook database in 12314. The text is retrieved in a new table in 12316 for the sensor name, status, type; measurement applications; resolution; swath; and accuracy. The CEOS Handbook database also includes information on mission launch date, mission end of life date; orbit details, etc. The names of the satellite missions from the newly retrieved table in the Level 1 key word search are used as key words in a new Level 2 key word in 12318. The search and retrieval in 12312 and 12314 are run again for the Level 2 key words. The mission launch data and end of life date is retrieved to the output of the table in 12320, and is combined with the Level 1 outputs. Other information related to the cost of image acquisition and planned mission continuity is retrieved by the user from the website of the sponsoring Earth Observation Agency. This additional information is retrieved via the internet and added to the satellite instrument table with the results of the Level 1 and Level 2 text retrieval from the CEOS Handbook and as other relevant information become available online. The instrument table is saved in electronic format to Database 2 in 12322 and updated with new releases for the CEOS Handbook. In 12324, the Level 1 and Level 2 key words are stored on a meta-database on Database 2. The instrument table is scored and ranked for a client's monitoring requirements, such as: 1) whether the instrument has global reach for annual monitoring, 2) has at least 5 years of annual historical data to develop a baseline for vegetation attribute(s) at a project site, 3) has data continuity for at least the next 10 years for post-validation monitoring of a vegetation attribute to be consistent with the baseline, and 4) the cost of image acquisition.

FIG. 4B shows an illustrative output for the strategic assessment of current and planned satellite missions and instrument sensor capabilities based on data continuity and data provision for a client's project offset activity. In 12502, t₀-t₄₀ an example of 41 years in time is shown. This amount of years is only given as an example and the amount of years is variable and dependent on the client's project lifetime. In 12504, an initial baseline period is shown during which a client will need to obtain measurements of a vegetation attribute. The example of the initial baseline in 12504 is for 10 years (i.e., t₀-t₉) and is used to develop the initial baseline for the vegetation attribute. Normally, the initial baseline can be developed for a period of 5 to 10 years prior to the project start date, because the initial baseline needs to assess the status of current vegetation attributes under the current land use regime prior to project implementation. A longer baseline might be considered inappropriate because it could possibly represent a more historic land use that is no longer reality on the ground. It is important to note here that the project developer client cannot monetize credits for the initial baseline, because this period represents time before the project implementation period. In 12506, a predicted project offset period is shown. 12506 is the offset crediting period for the clients project. The predicted offset is calculated by the client from the baseline assessment with fractional reductions of GHG emissions derived from the project activity. The example in 12506 shows a predicted offset for a project activity lasting 40 years (i.e., between t₁ and t₄₀). In 12508, the project validation year is shown in year t₁₀. 12508 is the year that the client intends to get the project accredited through a trading mechanism, or if the project was already accredited, is the year in which the project was accredited. This also means that the baseline in 12504 will normally be developed from a historical database prior to the date of validation. The project may need to be re-validated later in the project life to re-assess a future baseline, but an example of this re-assessment is not provided here. In 12510, project verification for real offsets is shown at 5 year intervals. The 5 year interval is only used as an example here and is dependent on the client. In 12512, a period is shown that matches 12506, where 12512 is the actual annual monitoring used to assess a vegetation attribute after the baseline period during the project implementation. The point is that the actual annual monitoring in 12512 is completed to show that the project activity is actually removing the GHGs that were predicted in 12506. The information contained in 12502 through 12512 is fully dependent on the client's project activity specifications. The point of 15202 through 12512 is to show how many years of continuous monitoring data the client needs to make a project work in reality as a tradable commodity. In 12518, the full period of data provision requirements is shown for the period t₀ through t₄₀. The point of 12518 is that data provision for a project site should be consistent for the entire period shown in 12518. In 12514, multiple satellite sensors for current and future satellite missions are shown that will be used to suggest the most appropriate data continuity to monitor a client's project site. The period going forward in time is limited to what is known about future planned satellite missions. In 12514, an example of 6 current and future planned sensors is used as an example. The 6 sensors all have very similar sensors (i.e., resolution, swath, and is either active and/or passive in information collection). In 12516, the satellite mission project life is shown for each of the 6 sensors. 12516 shows that all years need to be accounted for matching the full period shown in 12518. For data continuity, there may be overlaps in time between sensors, but typically there would not be substantial missing periods in time. If the project lifetime goes beyond the available information for current and planned sensors, the longest possible data continuity is shown. Data continuity can be more important than any other instrument sensor characteristic (i.e., resolution and/or whether the sensor is active or passive) for effective project monitoring and/or reporting, because a satellite might only have a mission life of between 5-10 years when the client's project life can be in excess of 30 years. If a sensor is chosen that has no long-term data continuity, the client's investment for initial project development for the baseline vegetation attribute will be wasted. This is because the data/information source used to develop the baseline and the predicted offset will no longer be available to the client to show that the predicted offset actually happened once the satellite mission life has ended.

Based on the results from the ranking of information from the instrument table, a standard science product and database is chosen to be initially developed with the NASA Earth Observing System (EOS) Moderate Resolution Imaging Spectroradiometer (MODIS) instrument (i.e., sensor). The MODIS imagery is used to monitor all elements of a vegetation attribute (i.e., biophysical and land classification), because the MODIS class sensors can provide global annual temporal replication. The legal/policy review found that the use of allometric equations and processed-based ecosystem models are defined as Tier 3 significance (i.e., the highest ordered Tier) for monitoring biophysical elements of vegetation attributes. The science plan will formulate directions for allometric equations to be implemented with MODIS sensor imagery and/or a similar sensor class to the MODIS imagery in operation or planned (i.e., MERIS on the ENVISAT mission, VIIRS on the NPOESS mission and/or Sentinel-3). The biophysical elements of a vegetation attribute are developed for annual vegetation flux amount (i.e., vegetation growth) and annual storage in a vegetation stock amount (i.e., woody biomass). A secondary database is developed to provide higher resolution imagery support to a client, but at temporal replication of about once every 5-10 years. The secondary database is initially developed with Landsat imagery. The Landsat imagery is used to monitor land classification elements for a vegetation attribute only, because there is generally only one useful high quality image snapshot available on a global basis about once every 5-10 years with the Landsat class sensor. Other Landsat resolution class remote sensing imagery (i.e., from active and/or passive sensors) that is not free, can be obtained and a database developed at the request of a client with the costs associated to the acquisition of the imagery passed on to the client.

FIG. 4C shows the process used to retrieve text from publications for relevant science on monitoring vegetation attribute(s) with the identified satellite instrument. An intelligence assessment is gathered on the current knowledge base related to the targeted instrument sensor (i.e., the MODIS science base). The websites for publishers of peer-review journal articles are accessed with a computer work station. Examples of these websites are ScienceDirect and Wiley InterScience. The websites for the publishers of peer-review journal articles have internal key word search engines. The website key word search engines are used to search for the identified instrument (i.e., “MODIS”). All journal articles are downloaded and stored to Database 2. Text retrieval software is used to retrieve information from the peer-reviewed journal articles and the process is exemplified in FIG. 4C. A user accesses a computer workstation in 12402 that includes screen display(s), processor(s), hard drive(s), a keyboard, a mouse, a router connected to the internet and other physical elements related to a computer workstation, etc. A computer workstation 12402 in this context can store, retrieve, process and/or output data and/or communicate with other computers. The computer workstation is used to access text retrieval software in 12404 and word processing software in 12408. In 12406, the text retrieval software is used to access the meta-database for the key words develop by the legal policy analysis. In 12410, the key word search developed from Legal/Policy Analysis in 12234 and 12242 stored on a meta-database on Database 1 is used as a Level 1 key word search in 12410. The key words are entered into the text retrieval software in 12412, which is then processed for the target journal article in 12414. The retrieved outputs are sentences from the targeted journal article inputted to word processing software in 12416. If any new key words are identified from the text retrieval in 12416 by a user, the identified words are used as Level 2 key words in 12418. The Level 2 key words are then entered into the text retrieval software in 12412 and processed for the journal article. Tables and figures are also retrieved in any of the retrieval searches when the tables and figures are described by a key word. The retrieved outputs are sentences from the targeted journal article inputted to word processing software in 12420. The retrieved information in the word processing software is saved in electronic format to Database 2 in 12422 and updated with the availability of new journal articles. In 12424, the Level 1 and Level 2 key words are stored on a meta-database on Database 2.

The NASA MODIS sensor's land team has developed many very high quality standard products relevant to monitoring the earth's surface, but not one of these standard products meets the specific legal requirements that are specified by the legal/policy analysis. For example, MODIS MOD 17 was developed to monitor global carbon sequestration for Gross Primary Production (GPP) and Net Primary Production (NPP), but neither the GPP nor NPP cycle measurements fulfill the annual reporting requirements from the legal/policy analysis. The standard MODIS products were developed to meet research needs for an academic audience and user group. The MODIS standard products are, however, very useful as a science-base for which new extensions can be developed that specifically meet compliance guidance for monitoring and/or reporting from the legal/policy analysis. Methods described as Allometric Equations 1 are used to extend standard remote sensing products, such as MODIS MOD 17, with a processed-based dynamic ecosystem, such as Biome-BGC, to a new vegetation attribute amount that complies with the required amount for monitoring and/or reporting of a vegetation attribute noted in the legal/policy review.

IPCC GPG-LULUCF (IPCC, 2003a) stated Biome-BGC as “an example” of a dynamic ecosystem process model (i.e., a biogeochemical model) that can be used in independent verification of vegetation attributes. Biome-BGC is an ecosystem process model that estimates storage and flux of carbon, nitrogen and water. Theory and applications of Biome-BGC and its predecessor, FOREST-BGC, are widely available (e.g., Hunt et al. 1996; Kimball et al. 1997a; Kimball et al. 1997b; Running 1994; Running and Coughlan 1988; Running and Gower 1991; Running and Hunt 1993; Running and Nemani 1991; White et al. 1999; White et al. 2000, Mu et al. 2008). Biome-BGC is available online for download here: <URL: http://www.ntsg.umt.edu/models/bgc/>.

NASA MODIS MOD 17 product for Gross Primary Production (GPP) and Net Primary Production (NPP) was the first continuous satellite-driven dataset monitoring global vegetation productivity (See MODIS MOD 17 user guide, a copy of which is incorporated herein by reference). The MODIS MOD 17 user guide can also be retrieved from: <URL: http://www.ntsg.umt.edu/modis/MOD17UsersGuide.pdf>. The modeling architecture for MOD 17 was developed around Biome-BGC. MOD 17 outputs are validated with FLUXNET data [available online at: <URL: http://www.fluxnet.ornl.gov/fluxnet/index.cfm>]. The difference between Biome-BGC modeled GPP/NPP and MOD 17 modeled GPP/NPP is that the MOD 17 outputs represent real-world growth rates after disturbance and the Biome-BGC outputs are theoretical growth rates before disturbance. Thus, a combination of MODIS GPP/NPP and newly developed Allometric Equations 1 from Biome-BGC modeled parameters extended to MOD 17 are used to quantify all carbon flux (i.e., sequestration or vegetation growth) and storage required for annual GHG reporting in the IPCC GPGs.

Allometric Equations 1 are used to develop annual carbon flux vegetation attribute by extending the modeling architecture used to develop MOD 17 from Biome-BGC. The new extensions use the outputs from the legal/policy review that define the following: the carbon cycle; the carbon pools within the carbon cycle; and the equations used to calculate the annual carbon pools within the carbon cycle. Allometric Equations 1 start at the carbon cycle variable for Net Primary Production (NPP), where MODIS MOD 17 ends. The IPCC's definitions state the amount from NPP after disturbance and respiration is the required annual carbon stock change amount reported in five carbon pools: above-ground biomass, below ground biomass, dead wood, litter and soil organic matter. The IPCC also states that the flux in the carbon pool lost to harvested wood products (i.e., carbon stocks lost to deforestation/degradation) is reported separately. Thus, NPP is 100% of the total amount of carbon that can be allocated to the five carbon pools and lost to respiration. Carbon sequestration from vegetation growth after disturbance is the difference between remote sensing-derived NPP and a process-based dynamic ecosystem model-derived NPP. The average percentages of Biome-BGC modeled NPP allocated annually to the five carbon pools and lost to respiration is used to develop Allometric Equations 1 for annual carbon flux (i.e., carbon sequestration or vegetation growth rates). Allometric Equations 1 meet the same specifications of the MOD 17 architecture (i.e., the fractions change by land cover type). Allometric Equations 1 for annual carbon flux are then used with MOD 17 NPP to partition the total remote sensing NPP to the five carbon pools after carbon is lost to respiration.

Allometric Equations 1 are developed for the annual woody biomass/carbon stock storage in a vegetation attribute by extending the modeling architecture used to develop MOD 17 from Biome-BGC. The new Biome-BGC extensions for annual woody carbon stocks are based on the theoretical architecture of MOD 17 available online at: <URL: http://www.fluxnet.ornl.gov/fluxnet/index.cfm>. One element in the MOD 17 process for calculating NPP includes a calculation for the vegetation attribute of annual live-wood biomass from a vegetation index (i.e., Leaf-Area Index [LAI] in standard publically disclosed MOD 17 architecture). The term live-wood biomass means the outer bark, inner bark, cambium and sapwood portions of a tree's physiology. The dead wood biomass element of the tree's physiology is still needed to calculate annual woody biomass storage. The term dead wood means the heartwood of a tree's physiology. In theory, total woody biomass storage is 100%. Live wood biomass and dead wood biomass equals a percentage allocation of the total woody biomass at 100%. Biome-BGC is used to model annual woody biomass, annual live wood biomass and annual dead wood biomass. Allometric Equations 1 for woody biomass are then developed for the fractional relationship between annual woody biomass stocks, annual live wood biomass and annual dead wood biomass. Allometric Equations 1 meet the same specifications of the MOD 17 architecture (i.e., the fractions change by land cover type). Allometric Equations 1 for annual woody biomass storage are then used with MOD 17-derived live wood biomass to equate annual dead wood biomass that is in turn used with live wood biomass to equate annual total woody biomass storage.

Data mining software uses input data and machine learning algorithms to extract patterns from the input data that are used to develop a predictor model. The methods described for Allometrics Equations 2 use data mining software to develop a regression and/or classification predictor model between a targeted sample(s) of a vegetation attribute and remote sensing imagery. The input data is stored on the database and comprises of 1) samples of target data for geospatial vegetation attributes and 2) remote sensing imagery (i.e., the standard MODIS database, the secondary Landsat database and/or other remote sensing imagery that a client prefers to use). A standard data mining target database is developed for samples of vegetation attribute(s) from any and/or all of the following sources: 1) geospatial data recorded as a publication in peer-review literature (i.e., this means the text of the publication provides a vegetation attribute amount with an associated longitude and latitude in the text of the publication and/or when there is a referenced publication to a publically available gridded electronic geospatial data file of a vegetation attribute); 2) geospatial data of the vegetation attribute reported publically as an official estimate from a government body; 3) geospatial data of the vegetation attribute reported publically that is stored on the database of a regulated and/or voluntary trading mechanism; 4) the standard remote sensing modeled product(s) for a vegetation attribute, such as MODIS MOD 17 and 5) any and/or all other publically available geospatial data for a vegetation attribute that is relevant to a client for monitoring and/or reporting under a trading mechanism. A client can also provide confidential project specific geospatial data for a vegetation attribute(s) that is stored in a database. The data mining software mines the target samples of vegetation attributes with the remote sensing imagery stored on the database. The mining identifies whether a pattern exists between the samples of the vegetation attribute and the remote sensing imagery. A pattern that exists between the samples for the vegetation attribute and remote sensing imagery is used to train a predictor model. Model validation and verification options are standard in data mining software, so the percentage of samples reserved for validation and/or verification is decided by the client. After an acceptable predictive model is developed, the data mining software is used to score the full image of input remote sensing data for a predicted vegetation attribute.

FIG. 4D shows the generic process used to develop a science plan. A computer workstation in 402 includes screen display(s), processor(s), hard drive(s), a keyboard, a mouse, a router connected to the internet and other physical elements related to a computer workstation, etc. A computer workstation for 402 in this context can store, retrieve, process and/or output data and/or communicate with other computers. Copyright 1 from 238 is viewed in 404 as either a printed out hard copy and/or accessed in Portable Document Format (i.e, .pdf and/or similar file format) by the computer workstation. A word processing software (i.e., MS Word) is accessed in 406 by the computer workstation. The text from the legal/policy analysis is pulled by the user in 408 related to what is required for monitoring the vegetation attribute. The retrieved text from the intelligence analysis from steps 12416 and 12420 is accessed in 410, edited and synthesized by a user to summarize the current knowledge base. In 412, the directions that will be used to develop and implement the monitoring procedures for a vegetation attribute are drafted by the user. The directions in 412 include specific equations, methods, software and input data that will be used to monitor the vegetation attribute. The full document is saved on Database 2 in 414. The outputs of 412 are accessed in 416, defined as Copyright 2 in 418 that is printed out in either a Portable Document Format (i.e, .pdf and/or similar file document format) digital file and/or in hard copy with a printer in 420.

FIG. 4E shows an example for the generic process used to develop a science plan applied to the outputs of 10238. Science plans are developed to match the technical requirements specified in the Legal/Policy Analysis for monitoring and/or reporting the relevant vegetation attributes—carbon flux and storage—that are in line with the Voluntary Carbon Standard's (VCS) guidance in AFOLU and the Climate, Community, and Biodiversity Alliance's Standards. A computer workstation in 10402 includes screen display(s), processor(s), hard drive(s), a keyboard, a mouse, a router connected to the internet and other physical elements related to a computer workstation, etc. A computer workstation at 10402 in this context can store, retrieve, process and/or output data and/or communicate with other computers. Copyright 1 from 10238 is stored and managed as a data structure, and, for example, can be viewed in 10404 as either a printed out hard copy and/or accessed in Portable Document Format (i.e., .pdf and/or similar file format) by the computer workstation. A word processing software (i.e., MS Word) is accessed in 10406 by the computer workstation. The text from the legal/policy analysis is pulled by the user in 10408 related to what is required for monitoring the vegetation attribute. The retrieved text from the intelligence analysis from steps 12416 and 12420 is accessed in 10410, edited and synthesized by a user to summarize the current knowledge base. In 10412, the directions that will be used to develop and implement the monitoring procedures for a vegetation attribute are drafted by the user. The directions in 10412 include specific equations, methods, software and input data that will be used to monitor the vegetation attribute. The full document is saved on Database 2 in 10414. The outputs of 10412 are accessed in 10416, defined as Copyright 2 in 10418 that is printed out in either a Portable Document Format (i.e., .pdf and/or similar file document format) digital file and/or in hard copy with a printer in 10420.

The following from points 1-5 are examples of science plans and defined as Copyright 2 in 10418 from FIG. 4E, which is/are generated for monitoring the target vegetation attribute for the target eco-region, based upon the compiled policy parameters.

1.0 Policy background (the text in section 1 is retrieved text referenced in 10408 from FIG. 4E and is similar as the legal/policy analysis, except the material in Table 1. Policy guidance in section one is only provided as a justification for land use in Table 1 justified):

1.1 Use of standardized international land cover data sets:

1.2 The use of international land cover date sets can be used to monitor the 6 land use categories required in the AFOLU sector in the following capacity (IPCC, 2006, Vol. 4, Ch 3, p. 3.25):

1.3 Estimating spatial distribution of land-use categories: Conventional inventories usually provide only the total sum of land-use area by classes. Spatial distribution can be reconstructed using international land-use and land cover data as auxiliary data where national data are not available.

1.4 Reliability assessment of the existing land-use datasets: Comparison between independent national and international datasets can indicate apparent discrepancies, and understanding these may increase confidence in national data and/or improve the usability of the international data, if required for purposes such as extrapolation.

1.5 When using an international dataset, inventory compilers should consider the following:

1.6 The classification scheme (e.g., definition of land-use classes and their relations) may differ from that in the national system.

1.7 Spatial resolution (typically 1 km nominally but sometimes an order of magnitude more in practice) may be coarse, so national data may need aggregating to improve comparability.

1.8 Classification accuracy and errors in geo-referencing may exist, though several accuracy tests are usually conducted at sample sites. The agencies responsible should have details on classification issues and tests undertaken.

1.9 As with national data, interpolation or extrapolation will probably be needed to develop estimates for the time periods to match the dates required for reporting.

1.10 The IPCC Good Practice Guidance suggested that international land cover maps can be used to monitor the six land use categories required in the AFOLU sector (IPCC, 2006, Vol. 4, Ch 3, p. 3.25). The IPCC GPG-2006 referred to the International Geosphere-Biosphere Program's (IGBP) Global 1 km×1 km land cover map as one example of international land cover maps applicable for comparison with national datasets (IPCC, 2006, Vol. 4, Ch 3, p. 3.25). Carbon Auditors used IGBP land cover classification maps developed from the NASA EOS MODIS satellite sensor to reclassify and map the six AFOLU categories. Table 1 lists the IGBP land cover classes in the left column. The right column lists which of the IGBP land cover classes were reclassified to AFOLU classes for the following: forested land, grassland, cropland, wetland, settled land and other land.

TABLE 1

| IGBP Definitions | AFOLU definitions |
|---|---|
| 0) Water | No Data |
| 1) Evergreen needleleaf forest | 1) Forested land |
| 2) Evergreen broadleaf forest | 1) Forested land |
| 3) Deciduous needleleaf forest | 1) Forested land |
| 4) Deciduous broadleaf forest | 1) Forested land |
| 5) Mixed forests | 1) Forested land |
| 6) Closed shrublands | 2) Grassland |
| 7) Open shrublands | 2) Grassland |
| 8) Woody savannas | 2) Grassland |
| 9) Savannas | 2) Grassland |
| 10) Grasslands | 2) Grassland |
| 11) Permanent wetlands | 4) Wetland |
| 12) Croplands | 3) Cropland |
| 13) Urban and built-up | 5) Settled land |
| 14) Cropland/natural vegetation mosaic | 3) Cropland |
| 15) Permanent snow and ice | 6) Other land |
| 16) Barren or sparsely vegetated | 6) Other land |
| 17) UNCLASSIFIED | 6) Other land |

1.11 Carbon Model Theory & Design (the text in section 1.11 is retrieved from the intelligence assessment from FIG. 4C):

Biome-BGC

Biome-BGC is an ecosystem process model that estimates storage and flux of carbon, nitrogen and water. Using prescribed site conditions, meteorology, and parameter values, Biome-BGC simulates daily fluxes and states of carbon, water, and nitrogen for coarsely defined biomes at areas ranging from 1 m² to the entire globe. Plant physiological processes respond to diurnal environmental variation (Geiger and Servaites 1994), but Biome-BGC uses a daily time in order to take advantage of widely available daily temperature and precipitation data from which daylight averages of short wave radiation, vapor pressure deficit, and temperature are estimated (Thornton et al. 1997; Thornton and Running 1999).

Biome-BGC simulates the development of soil and plant carbon and nitrogen pools; no input of soil carbon information or leaf area index (LAI, m² leaf area per m² ground area) is required. LAI controls canopy radiation absorption, water interception, photosynthesis, and litter inputs to detrital pools and is thus central to Biome-BGC. Model structure is discussed by Thornton (Thornton, 1998), and will not be presented here. Briefly, though, NPP is based on gross primary production simulated with the Farquhar photosynthesis model (Farquhar et al. 1980) minus maintenance respiration [calculated as a function of tissue nitrogen concentration (Ryan 1991)] and growth respiration (a constant fraction of gross primary production). Theory and applications of Biome-BGC and its predecessor, FOREST-BGC, are widely available (e.g., Hunt et al. 1996; Kimball et al. 1997a; Kimball et al. 1997b; Running 1994; Running and Coughlan 1988; Running and Gower 1991; Running and Hunt 1993; Running and Nemani 1991; White et al. 1999; White et al. 2000, Mu et al. 2008).

NASA MODIS MOD 17

NASA MODIS MOD 17 product for Gross Primary Production (GPP) and Net Primary Production (NPP) is the first continuous satellite-driven dataset monitoring global vegetation productivity. The algorithm is based on the original logic of Monteith (1972, 1977) suggesting that NPP under non-stressed conditions is linearly related to the amount of absorbed Photosynthetically Active Radiation (PAR) during the growing season. In reality, vegetation growth is subject to a variety of stresses that tend to reduce the potential growth rate, especially stresses resulting from climate (temperature, radiation, and water) or the interaction of these primary abiotic controls, which impose complex and varying limitations on vegetation activity in different parts of the world (Churkina & Running, 1998; Nemani et al., 2003; Running et al., 2004). Combining the logic of Monteith, climate controls, and some principles of modeling NPP learned from a general process-based ecosystem model, Biome-BGC (Running & Hunt, 1993), the MODIS GPP/NPP algorithm was developed using satellite-derived land cover, the fraction of photosynthetically active radiation absorbed by vegetation (FPAR), and leaf area index (LAI) as input surface vegetation information (Running et al., 2000), while the necessary climate information is obtained from a global climatic data assimilation system developed by the NASA Goddard Global Modeling and Assimilation Office (GMAO).

2.0 Science Plan-Directions 1: Application of a dynamic ecosystem model to develop Allometric Equations 1 for carbon flux variables. (Section 2 through Section 5 in this example of a science plan are referenced in 10412 from FIG. 4D)

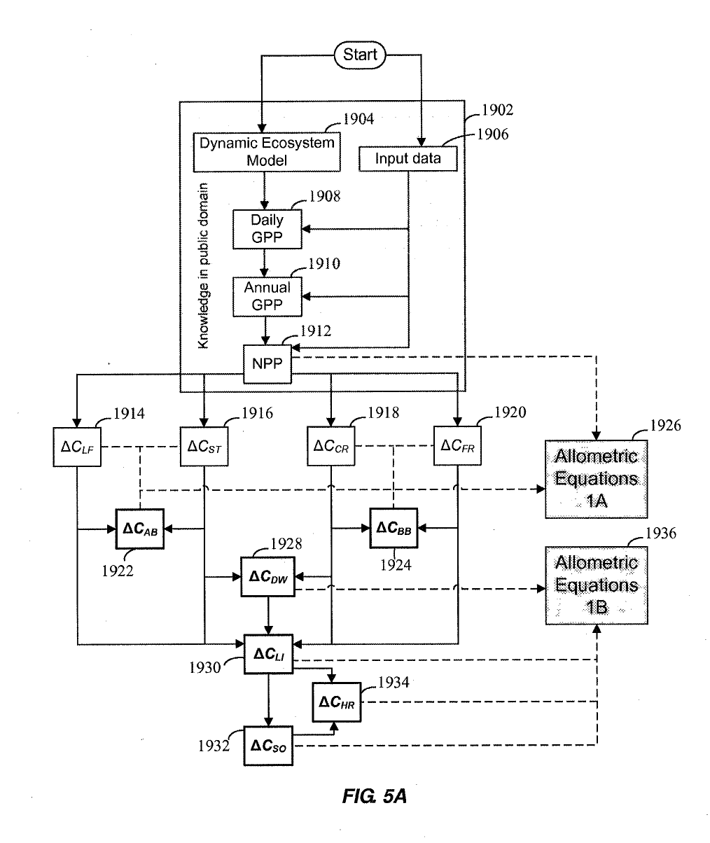

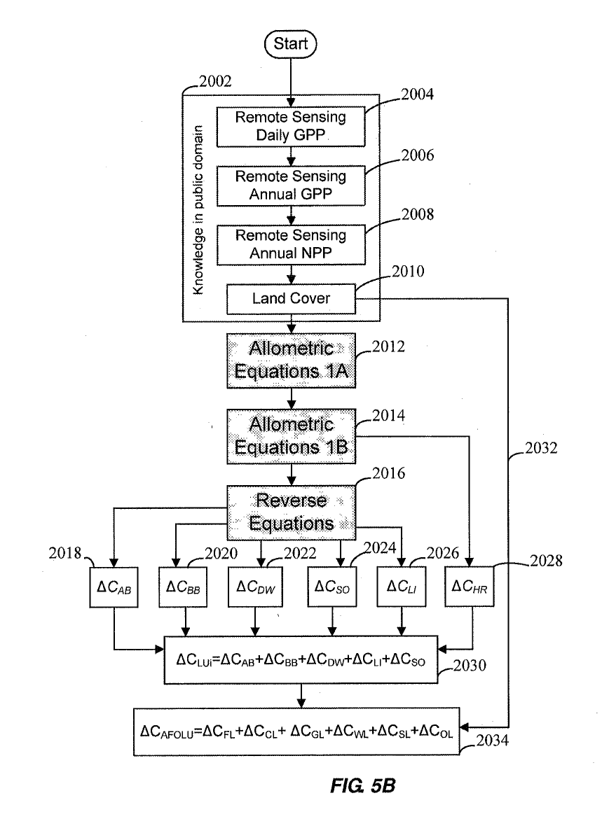

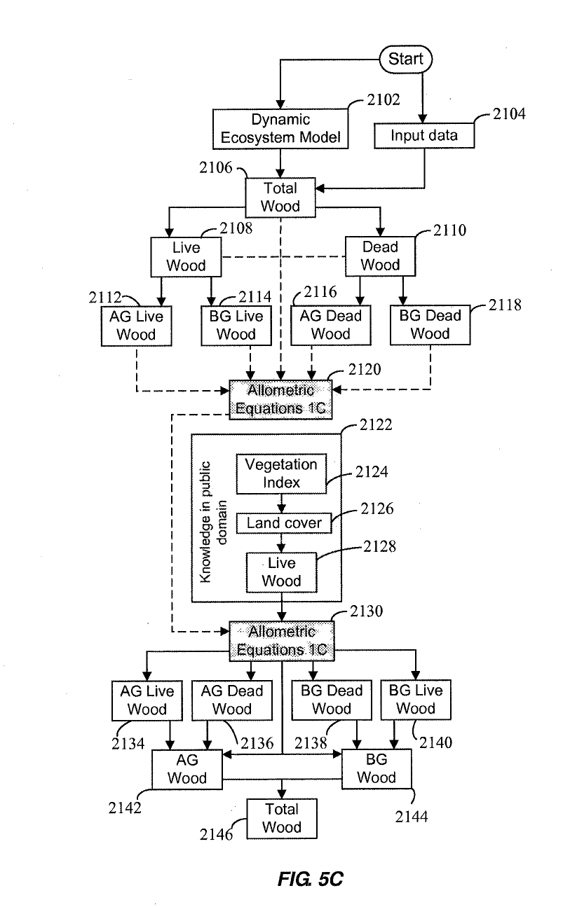

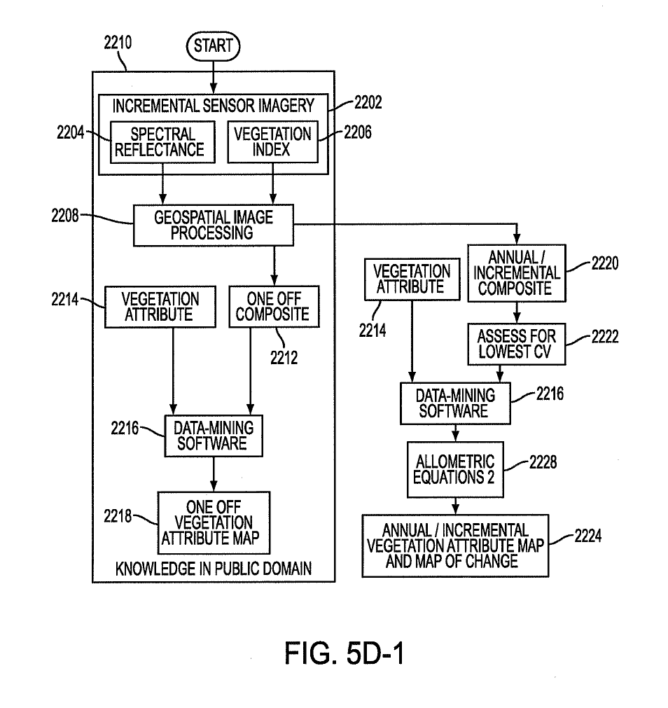

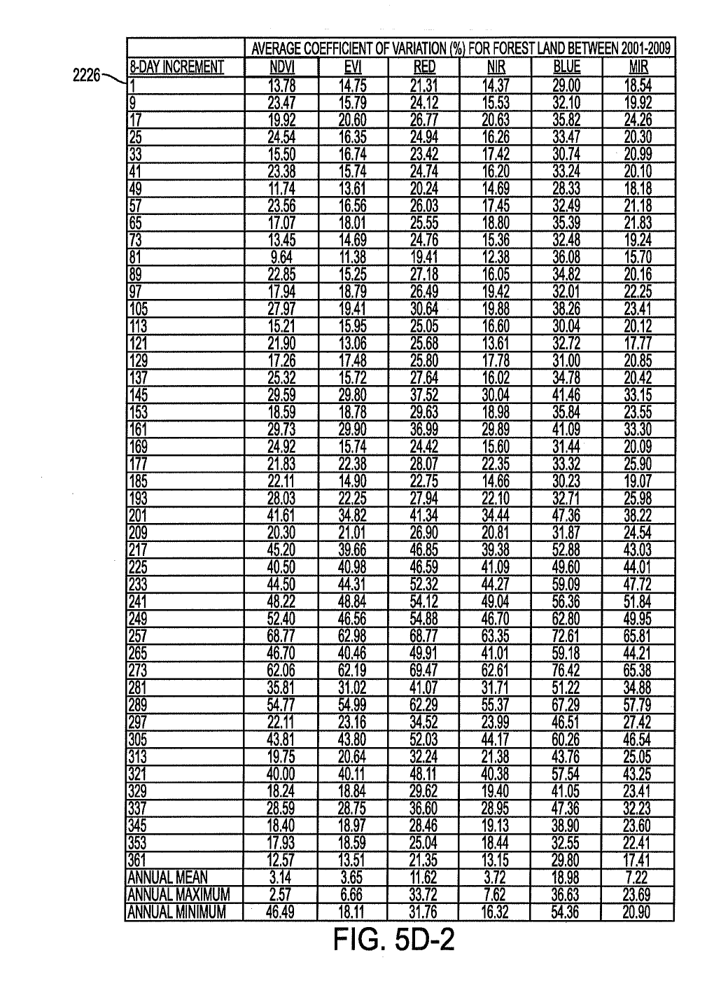

2.1FIG. 5A shows an example of the process used to develop Allometric Equations 1. FIG. 5A is used with Science Plan-Directions 1 in section 2 in the example of a science plan. Directions 1 develop allometrics from a processed-based dynamic ecosystem model for carbon sequestration. Straight lines show the pathway or route to calculate carbon flux and dotted lines indicate route to develop Allometric Equations 1. More particularly, the following are methods for implementing a dynamic ecosystem model with a land cover map to develop Allometric Equations for monitoring the legally required carbon pools identified for carbon flux. The allometrics that are developed are, in theory, an extension to modeling gross primary production (GPP) and net primary production (NPP) (i.e., a light use efficiency model) with remote sensing imagery. The allometrics can therefore be implemented with any remote sensing-derived model of NPP. The allometrics were specifically developed here as an extension to the NASA Moderate Resolution Imaging Spectroradiometer (MODIS) sensor standardized global GPP/NPP algorithm called MOD 17. In this example, the dynamic ecosystem model BIOME-BGC is run for thirty individual years with global input data and the mean carbon pool variable for all years will be used for development Allometric Equations 1. FIG. 5A shows the process used to develop carbon flux variables. The box 1902 defines the knowledge in public space. The steps that fall outside 1902 are new to science. The process begins by using input data in 1906 and a dynamic ecosystem model in 1904 to calculate daily GPP in 1908, annual GPP in 1910 and NPP in 1912.

2.2 Develop Allometric Equations 1A in 1926 (see FIG. 5A) for above-ground biomass (ΔC_{AB}) in 1922 is calculated as the sum of leaf organic matter (ΔC_{LF}) in 1914 and the sum of organic matter diverted to the stem (ΔC_{ST}) in 1916 as the following (AB₁ and AB₂):

LC_{i—}AB_{1—}LF_ratio=LCᵢ( χ̅ΔC_{LF} [gCm⁻²]/ χ̅NPP [gCm⁻²]) Eq.3

LC_{i—}AB_{2—}ST_ratio=LCᵢ( χ̅ΔC_{ST} [gCm⁻²]/ χ̅NPP [gCm⁻²]) Eq.4

Where LCᵢ is the specific land cover category (BGC land cover was used here), χ̅ΔC_{LF} is the mean carbon flux (g) in annual leaf organic matter for an individual land cover class (but is interchangeable with any land cover classification system), χ̅ΔC_{ST} is the mean carbon flux (g) in annual leaf organic matter for an individual land cover class and χ̅NPP is the annual carbon flux (g) amount for mean Net Primary Production (NPP) in the same land cover category.

2.3 Develop Allometric Equations 1A (see FIG. 5A) for below-ground biomass (ΔC_{BB}) in 1924 will be calculated as the sum of fine root carbon (ΔC_{FR}) in 1920 and the sum of coarse root carbon (ΔC_{CR}) in 1918 as the following (BB₁ and BB₂):

LC_{i—}BB_{1—}FR_ratio=LCᵢ( χ̅ΔC_{FR} [gCm⁻²]/ χ̅NPP [gCm⁻²]) Eq.5

LC_{i—}BB_{2—}CR_ratio=LCᵢ( χ̅ΔC_{CR} [gCm⁻²]/ χ̅NPP [gCm⁻²]) Eq.6

Where LCᵢ is the specific land cover category, χ̅ΔC_{FR} is the mean fine root carbon accumulation (g) amount for an individual land cover category, χ̅Δ_{CR} is the mean coarse root carbon accumulation (g) amount for an individual land cover category and χ̅NPP is the annual carbon flux (g) amount for mean Net Primary Production (NPP) in the same land cover category.

2.4 Develop Allometric Equations 1B in 1936 (see FIG. 5A) based on the combined relationship of biomass carbon pools allocated to Dead Orgainic Matter and Soil carbon pools defined in the generalized IPCC flowchart of the carbon cycle (see FIG. 5A). The allometric equations are based on the mean carbon flux values for all years of the model run.

Annual dead wood (ΔCow) accumulation in 1928 for each category is calculated by the following two calculations (DW₁ and DW₂):

LC_{i—}DW_{1—}ST_ratio=LCᵢ( χ̅ΔC_{DW} [gCm⁻²]/ χ̅ΔC_{ST} [gCm⁻²]) Eq.7

LC_{i—}DW_{2—}CR_ratio=LCᵢ( χ̅ΔC_{DW} [gCm⁻²]/ χ̅ΔC_{CR} [gCm⁻²]) Eq.8

Where LCᵢ is the specific land cover category, χ̅ΔC_{DW} is the mean annual dead wood accumulation in the associated land cover category, χ̅ΔC_{ST} is the mean carbon flux (g) in annual leaf organic matter for an individual land cover class, and χ̅ΔC_{CR} is the mean coarse root carbon accumulation (g) amount for an individual land cover class.

2.5 Annual litter (ΔC_{LI}) accumulation in 1930 for each category is calculated by the following five calculations (LI₁-LI₅):

LC_{i—}LI_{1—}LF_ratio=LCᵢ( χ̅ΔC_{LI} [gCm⁻²]/ χ̅ΔC_{LF} [gCm⁻²]) Eq.9

LC_{i—}LI_{2—}ST_ratio=LCᵢ( χ̅ΔC_{LI} [gCm⁻²]/ χ̅ΔC_{ST} [gCm⁻²]) Eq.10

LC_{i—}LI_{3—}FR_ratio=LCᵢ( χ̅ΔC_{LI} [gCm⁻²]/ χ̅ΔC_{FR} [gCm⁻²]) Eq.11

LC_{i—}LI_{4—}CR_ratio=LCᵢ( χ̅ΔC_{LI} [gCm⁻²]/ χ̅ΔC_{CR} [gCm⁻²]) Eq.12

LC_{i—}LI_{5—}DW_ratio=LCᵢ( χ̅ΔC_{LI} [gCm⁻²]/ χ̅ΔC_{DW} [gCm⁻²]) Eq.13

Where LCᵢ is the specific land cover category, χ̅ΔC_{LI} is the mean annual litter accumulation in the specific LCᵢ category, χ̅C_{LF} is the mean annual leaf carbon transferred to litter in the specific LCᵢ category, χ̅ΔC_{ST} is the mean annual stem carbon transferred to litter in the specific LCᵢ category, χ̅ΔC_{FR} is the mean annual fine root transferred to litter in the specific LCᵢ category, χ̅ΔC_{CR} is the mean annual coarse root transferred to litter in the specific LCᵢ category and χ̅ΔC_{DW} is the mean annual dead wood accumulation in the associated LCᵢ category.

2.6 Annual soil organic matter (ΔC_{SO}) accumulation in 1932 for each category is calculated by the following equation (SO₁):

LC_{i—}SO_{1—}LI_ratio=LCᵢ( χ̅ΔC_{SO} [gCm⁻²]/ χ̅ΔC_{LI} [gCm⁻²]) Eq.14

Where LCᵢ is the specific land cover category, χ̅ΔC_{SO} is the mean annual accumulation in soil organic matter in the individual LCᵢ category and χ̅ΔC_{LI} is the mean annual litter accumulation in the specific LCᵢ category.

2.7 Annual heterotrophic respiration (ΔC_{HR}):

Heterotrophic respiration is the carbon flux amount loss to the atmosphere due to decomposition. The IPCC GPGs defines Net Ecosystem Production (NEP) as the numerical difference between heterotrophic respiration and Net Primary Production (NPP) (section 1.5 above). The IPCC GPGs only suggest using the mean rate of regional decay and do provide direction for calculating heterotrophic respiration set forth by the generic equations in the GPG-2006 (Equation 2 above). Two methods for measuring heterotrophic respiration can be used. The first is directly using the annual heterotrophic respiration measurements from a dynamic process model. This method is useful for monitoring dynamic annual heterotrophic respiration. The second is to develop an average heterotrophic respiration amount, so that heterotrophic respiration can be spatially assessed at the same resolution as the satellite imagery. Section 2.8 describes the second method to equate mean heterotrophic respiration.

2.8 Annual heterotrophic respiration (ΔC_{HR}) accumulation in 1934 for each land cover category is calculated by the following two calculations (HR₁ and HR₂):

LC_{i—}HR_{1—}LI_ratio=LCᵢ( χ̅ΔC_{HR} [gCm⁻²]/ χ̅ΔC_{LI} [gCm⁻²]) Eq.15

LC_{i—}HR_{2—}SO_ratio=LCᵢ( χ̅ΔC_{HR} [gCm⁻²]/ χ̅ΔC_{SO} [gCm⁻²]) Eq.16

Where LCᵢ is the specific land cover category, χ̅ΔC_{HR} is the mean annual dead wood accumulation in the associated land cover category, χ̅ΔC_{LI} is the mean carbon flux in annual litter for an individual land cover class, and χ̅ΔC_{SO} is the mean soil organic matter carbon accumulation amount for an individual land cover category.

2.9 Application of MOD 17 to AFOLU requirements:

FIG. 5B shows an example of the process used to implement Allometric Equations 1 with remote sensing imagery. FIG. 5B is used with Science Plan-Directions 1 in section 2 in the example of a science plan. Directions 1 implement the allometrics from developed in FIG. 5A with remote sensing imagery. More particularly, the following methods are used to implement allometic Equations A and B with a satellite remote sensing dynamic model of Gross Primary Production (GPP) and Net Primary Production (NPP). FIG. 5B shows the process used to implement Allometric Equations 1A and 1B with remote sensing derived GPP/NPP and a standardized land cover map(s). Biome-BGC and MODIS MOD 17 were used as the dynamic ecosystem model from sections 2.1-2.8, but the process is applicable to all dynamic process models. Furthermore, the process in FIG. 20 can be implemented with any remote sensing measurement of GPP/NPP and/or any light use efficiency model. MODIS MOD 17 NPP algorithm provides real time and real world estimates of global carbon flux in biomass. The MOD 17 algorithm architecture is built around the BIOME-BGC model, which was cited in GPG-LULUCF as an example of well known ecosystem model that could be used for verification in the LULUCF sector under IPCC Good Practice Guidelines. Thus, a combination of MOD 17 NPP plus BIOME-BGC parameters for carbon pools fulfils the good practice guidance for LULUCF sector verification within the same dynamic ecosystem modeling architecture.

2.10 The box referred to in 2002 (see FIG. 5B) defines the current knowledge space in public domain. MOD 17 is run to calculate daily GPP in 2004, annual GPP in 2006 and NPP in 2008.

2.11 A standardized land cover map in 2010 is overlaid over NPP.

2.12 Implement Allometric Equations 1A in 2012 (see FIG. 5B):

2.13 The results for Equations 3 and 4 are processed with satellite derived NPP to determine total annual above-ground biomass flux (ΔC_{AB}):

ΔC_{AB—}LF_{—}1 [gCm⁻²]=LC_{i—}AB_{1—}LF_ratio*MOD17_NPP [gCm⁻²] Eq.17

ΔC_{AB—}ST_{—}2 [gCm⁻²]=LC_{i—}AB_{2—}ST_ratio*MOD17_NPP [gCm⁻²] Eq.18

ΔC_{AB—}IN_{—}3 [gCm⁻²]=ΔC_{AB—}LF_{—}1 [gCm⁻²]+ΔC_{AB—}ST_{—}2 [gCm⁻²] Eq.13

Where ΔC_{AB—}IN_{—}3 [gCm⁻²] is equal to the total carbon flux in ΔC_{AB}, but not inclusive of carbon flux transferred to other carbon pools.

2.14 The results for Equations 5 and 6 are processed with satellite derived NPP to determine total annual below-ground biomass flux (ΔC_{BB}):

ΔC_{BB—}FR_{—}1 [gCm⁻²]=LC_{i—}BB_{1—}FR_ratio*MOD17_NPP [gCm⁻²] Eq.20

ΔC_{BB—}CR_{—}2 [gCm⁻²]=LC_{i—}BB_{2—}CR_ratio*MOD17_NPP [gCm⁻²] Eq.21

ΔC_{BB—}IN_{—}3 [gCm⁻²]=ΔC_{BB—}FR_{—}1 [gCm⁻²]+ΔC_{BB—}CR_{—}2 NPP [gCm⁻²] Eq.21

Where ΔC_{BB—}IN_{—}3 [gCm⁻²] is equal to the total carbon flux amount in annual below ground biomass, but not inclusive of carbon flux transferred to other carbon pools.

2.15 Implement Allometric Equations 1B in 2014 (see FIG. 5B):

2.16 The results for Equations 7 and 8 are processed with Equations 3 and 4 to determine the annual accumulation of dead wood (ΔC_{DW}) per LCᵢ categories:

ΔC_{DW—}ST_{—}1 [gCm⁻²]=LC_{i—}DW_{1—}ST_ratio*ΔC_{AB—}ST_{—}2 [gCm⁻²] Eq.23

ΔC_{DW—}CR_{—}2 [gCm⁻²]=LC_{i—}DW_{2—}CR_ratio*ΔC_{BB—}CR_{—}2 [gCm⁻²] Eq.24

ΔC_{DW—}IN_{—}3 [gCm⁻²]=ΔC_{DW—}ST_{—}1 [gCm⁻²]+ΔC_{—}2 [gCm⁻²] Eq.25

Where ΔC_{DW—}IN_{—}3 [gCm⁻²] is equal to the total carbon flux amount in annual dead wood debris, but not inclusive of carbon flux transferred to annual litter flux.

2.17 The result for Equations 9 to 13, 17 and 18, 20 and 21, and 23 and 24 are processed together to determine total annual litter carbon flux (ΔC_{L}) per LCᵢ categories:

ΔC_{LI—}LF_{—}1 [gCm⁻²]=LC_{i—}LI_{1—}LF_ratio*ΔC_{AB—}LF_{—}1 [gCm⁻²] Eq.26

ΔC_{LI—}ST_{—}2 [gCm⁻²]=LC_{i—}LI_{2—}ST_ratio*ΔC_{AB—}ST_{—}2 [gCm⁻²] Eq.27

ΔC_{LI—}FR_{—}3 [gCm⁻²]=LC_{i—}LI_{3—}FR_ratio*ΔC_{BB—}FR_{—}1 [gCm⁻²] Eq.28

ΔC_{LI—}CR_{—}4 [gCm⁻²]=LC_{i—}LI_{4—}CR_ratio*ΔC_{BB—}CR_{—}2 [gCm⁻²] Eq.29

ΔC_{LI—}DW_{—}5 [gCm⁻²]=LC_{i—}LI_{5—}DW_ratio*ΔC_{DW—}IN_{—}3 [gCm⁻²] Eq.30

ΔC_{LI—}IN_{—}6 [gCm⁻²]=ΔC_{LI—}LF_{—}1 [gCm⁻²]+ΔC_{LI—}ST_{—}2 [gCm⁻²]+ΔC_{LI—}FR_{—}3 [gCm⁻²]+ΔC_{LI—}CR_{—}4 [gCm⁻²]+ΔC_{LI—}DW_{—}5 [gCm⁻²] Eq.31

Where ΔC_{LI—}IN_{—}6 [gCm⁻²] is equal to the total carbon flux amount in annual litter, but not inclusive of carbon flux flux transferred from litter to soil organic matter and lost to the atmosphere through heterotrophic respiration.

2.18 The results for Equations 14 and 31 are processed together to determine total annual soil organic matter carbon flux (ΔC_{SO}) per LCᵢ categories:

ΔC_{SO—}IN_{—}1 [gCm⁻²]=LC_{i—}SO_{1—}LI_ratio*ΔC_{LI—}IN_{—}6 [gCm⁻²] Eq.32

Where ΔC_{SO—}IN [gCm⁻²] is equal to the total carbon flux amount in soil organic matter under IPCC GPGs, but not inclusive of the carbon flux lost to the atmosphere during heterotrophic respiration.

2.19 The results for Equations 15 and 16 are processed with Equations 31 and 32 to determine annual heterotrophic respiration (ΔC_{HR}) in 2028 per LCᵢ category (see FIG. 5b):

ΔC_{HR—}LI_{—}1 [gCm⁻²]=LC_{i—}HR_{1—}LI_ratio*ΔC_{LI—}IN_{—}6 [gCm⁻²] Eq.33

ΔC_{HR—}SO_{—}2 [gCm⁻²]=LC_{i—}HR_{4—}SO_ratio*ΔC_{SO—}IN_{—}1 [gCm⁻²] Eq.34

ΔC_{HR—}IN_{—}3 [gCm⁻²]=ΔC_{HR—}LI_{4—}1 [gCm⁻²]+ΔC_{HR—}SO_{—}2 [gCm⁻²] Eq.35

Where ΔC_{HR—}IN_{—}3 [gCm⁻²] is equal to the total carbon flux lost to the atmosphere through heterotrophic respiration.

2.20 Implement Reverse Equations in 2016 (see FIG. 5B):

2.21 Reverse equations are used to calculate total annual carbon flux for Net Biome Production under IPCC GPGs per land use type (ΔC_{LUi}):

2.22 Equation 2 in 2030 was described in the IPCC Good Practice Guideline to calculate Net Biome Production per land use type.

2.23 Annual carbon flux in soil organic matter (ΔC_{SO}) in 2024 per land use category (LUi) is calculated by the following:

ΔC_{SO—}LUi [gCm⁻²]=ΔC_{SO—}IN_{—}1 [gCm⁻²]−ΔC_{HR—}SO_{—}2 [gCm⁻²] Eq.36

2.24 Annual carbon flux in litter (ΔC_{LI}) in 2026 per land use category (LUi) is calculated by the following:

ΔC_{LI—}LUi [gCm⁻²]=ΔC_{LI—}IN_{—}6 [gCm⁻²]−ΔC_{HR—}LI_{—}1 [gCm⁻²]−ΔC_{SO—}IN_{—}1 [gCm⁻²] Eq.37

2.25 Annual carbon flux in dead wood (ΔC_{DW}) in 2022 per land use category (LUi) is calculated by the following:

ΔC_{DW—}LUi [gCm⁻²]=ΔC_{DW—}IN_{—}3 [gCm⁻²]−ΔC_{LI—}DW_{—}5 [gCm⁻²] Eq.38

2.26 Annual carbon flux in below ground biomass (ΔC_{BB}) in 2020 per land use category (LUi) is calculated by the following:

ΔC_{BB—}LUi [gCm⁻²]=ΔC_{BB—}IN_{—}3 [gCm⁻²]−ΔC_{LI—}FR_{—}3 [gCm⁻²]−ΔC_{LI—}CR_{—}4 [gCm⁻²]−ΔC_{DW—}CR_{—}2 [gCm⁻²] Eq.39

2.27 Annual carbon flux in below ground biomass (ΔC_{AB}) in 2018 per land use category (LUi) is calculated by the following:

ΔC_{AB—}LUi [gCm⁻²]=ΔC_{AB—}IN_{—}3 [gCm⁻²]−ΔC_{LI—}LF_{—}1 [gCm⁻²]−ΔC_{LI—}ST_{—}2 [gCm⁻²]−ΔC_{DW—}ST_{—}1 [gCm⁻²] Eq.40

2.28 Reclassify the land cover map in 2032 to the LULUCF/AFOLU definitions described in Table 1.

2.29 Implement Equation 2 in 2030.

2.30 Total carbon flux for all land use types in all AFOLU in 2034 is calculated by Equation 1.

3.0 Science Plan-Directions 2: Application of a dynamic ecosystem model to develop Allometric Equations 1C for wood carbon (i.e., biomass stocks) variables.

3.1 This is a general method to incorporate dynamic process modeling with remote sensing techniques to quantify wood carbon stocks. The newly developed wood carbon storage allometrics are implemented to model remote sensing-derived woody biomass stocks and/or carbon storage in wood. Carbon stored in wood is used to quantify CO₂ storage in wood.

3.2 FIG. 5C shows an example of the process used to develop Allometric Equations 1. FIG. 5C is used with Science Plan-Directions 2 in section 3 in the example of a science plan. FIG. 5C is an example of a flow chart used to develop Allometric Equations 1 for woody biomass stocks and implement the allometrics with remote sensing imagery. More particularly, FIG. 5C is a flow chart of the process used to measure standing biomass stocks (i.e., total wood). The steps taken in our carbon model are the following:

3.3 A dynamic model is used to calculate annual total wood biomass storage in dry matter in 2106.

LC_{i—TotalDM}_{—}C_ratio=LCᵢ( χ̅TotalWood [tDMha]/ χ̅TotalWood [tCha]) Eq. 41

Where LCᵢ is each land cover class (i); χ̅TotalWood [tDMha] is the total wood storage in tonnes of dry matter per hectare for each land cover class; χ̅TotalWood [tCha] is the total wood storage in tonnes of carbon per hectare for each land cover class; and TotalDM_C_ratio is the ratio between total wood storage in tonnes of dry matter per hectare to total wood storage in tonnes of carbon per hectare.

3.4 Next a dynamic ecosystem model (see FIG. 5C) is used to calculate annual above-ground live wood biomass storage in dry matter in 2112, annual below-ground live wood biomass storage in dry matter in 2114, annual above-ground dead wood biomass storage in dry matter in 2116 and annual below-ground wood dead biomass storage in dry matter in 2118. Total live wood in 2108 and dead wood in 2110 are summed for above and below ground partitions of total wood. The term live wood means the outer bark, inner bark, cambium and sapwood portions of a tree's physiology. The term dead wood means the heartwood of a tree's physiology.

3.5 Allometric Equations 1C are developed to quantify the proportional relationship between total wood biomass in 2106, total above-ground live wood biomass in dry matter in 2112, below-ground live wood biomass in dry matter in 2114, above-ground dead wood biomass in dry matter in 2116 and below ground dead wood biomass in dry matter in 2118.

LC_{i—}AG_LW_ratio=LCᵢ( χ̅AG_LW [tDMha]/ χ̅TotalLW [tDMha]) Eq. 42

LC_{i—}BG_LW_ratio=LCᵢ( χ̅BG_LW [tDMha]/ χ̅TotalLW [tDMha]) Eq. 43

Where LCᵢ is each land cover class (i); χ̅AG_LW [tDMha] is the mean above ground live wood storage in tonnes of dry matter per hectare for each BGC land cover class; χ̅TotalLW [tDMha] is the mean total (above and below ground) live wood storage in tonnes of dry matter per hectare for each land cover class; χ̅BG_LW [tDMha] is the mean below ground live wood storage in tonnes of dry matter per hectare for each land cover class; LC_{i—}AG_LW_ratio is the ratio between above ground live wood storage in tonnes of dry matter per hectare to total live wood storage in tonnes of dry matter per hectare; and LC_{i—}BG_LW_ratio is the ratio between below ground live wood storage in tonnes of dry matter per hectare to total live wood storage in tonnes of dry matter per hectare.

LC_{i—}AG_DW_ratio=LCᵢ( χ̅AG_DW [tDMha]/ χ̅AG_LW [tDMha]) Eq. 44

LC_{i—}BG_LW_ratio=LCᵢ( χ̅BG_DW [tDMha]/ χ̅BG_LW [tDMha]) Eq. 45

Where LCᵢ is each land cover class (i); χ̅AG_LW [tDMha] is the mean above ground live wood storage in tonnes of dry matter per hectare for each BGC land cover class; χ̅AG_DW [tDMha] is the mean above ground dead wood storage in tonnes of dry matter per hectare for each BGC land cover class; χ̅BG_LW [tDMha] is the mean below ground live wood storage in tonnes of dry matter per hectare for each BGC land cover class; χ̅BG_DW [tDMha] is the mean below ground dead wood storage in tonnes of dry matter per hectare for each BGC land cover class; LC_{i—}AG_DW_ratio is the ratio between above ground dead wood storage in tonnes of dry matter per hectare to above ground live wood storage in tonnes of dry matter per hectare; and LC_{i—}BG_DW_ratio is the ratio between below ground dead wood storage in tonnes of dry matter per hectare to below ground live wood storage in tonnes of dry matter per hectare.

3.6 A remote sensing modeled vegetation index (i.e., leaf area index (LAI), enhanced vegetation index (EVI), and/or normalized difference vegetation index (NDVI)) is used calculate annual live wood mass (see FIG. 5C). The method to calculate live wood mass is found in the MODIS MOD 17 algorithm. The grey box in 2122 (see FIG. 21) defines the current knowledge in public domain. The dynamic ecosystem model derived allometric equations used to quantify total wood biomass for the following: annual above-ground live wood biomass storage in dry matter in 2134, annual below-ground live wood biomass storage in dry matter in 2138, annual above-ground dead wood biomass storage in dry matter in 2136 and annual below-ground wood dead biomass storage in dry matter in 2140.

AG_LW [tDMha]=LC_{i—}AG_LW_ratio*Tot_LW [tDMha]) Eq. 46

BG_LW [tDMha]=LC_{i—}BG_LW_ratio*Tot_LW [tDMha]) Eq. 47

Where LCᵢ is each land cover class (i); LC_{i—}AG_LW_ratio is the result for Equation 42; Tot_LW [tDMha] is derived from leaf area index or enhanced vegetation index for total live wood in tonnes of dry matter per hectare; LC_{i—}BG_LW_ratio is the result to Equation 43; AG_LW [tDMha] is the total above ground live wood storage in tonnes of dry matter per hectare, and BG_LW [tDMha] is the total below ground live wood storage in tonnes of dry matter per hectare.

AG_DW [tDMha]=LC_{i—}AG_DW_ratio*AG_LW [tDMha]) Eq. 48

BG_DW [tDMha]=LC_{i—}BG_DW_ratio*BG_LW [tDMha]) Eq. 49

Where LCᵢ is each land cover class (i); LC_AG_DW_ratio is the result for Equation 44; AG_LW [tDMha] is the total above ground live wood storage in tonnes of dry matter per hectare; LC_BG_DW_ratio is the result to Equation 45; BG_LW [tDMha] is the total below ground live wood storage in tonnes of dry matter per hectare; AG_DW [tDMha] is the total above ground dead wood storage in tonnes of dry matter per hectare, and BG_DW [tDMha] is the total below ground dead wood storage in tonnes of dry matter per hectare.

3.7 Annual total above-ground wood biomass storage in dry matter in 2142 and annual total below ground wood biomass storage in dry matter in 2144 are calculated next.

AG_Total [tDMha]=AG_LW [tDMha]+AG_DW [tDMha] Eq. 50

BG_Total [tDMha]=BG_LW [tDMha]+BG_DW [tDMha] Eq. 51

Where AG_LW [tDMha] is the result to Equation 46; AG_DW [tDMha] is the result to Equation 48, BG_LW [tDMha] is the result to Equation 47; BG_DW [tDMha] is the result to Equation 49; AG_Total [tDMha] is the total above ground wood storage in tonnes of dry matter hectare, and BG_Total [tDMha] is the total below ground wood storage in tonnes of dry matter hectare.

3.8 Annual total wood biomass storage in tonnes of dry matter in 2146 is calculated.

Total_wood [tDMha]=AG_Total [tDMha]+BG_Total [tDMha] Eq. 52

Where AG_Total [tDMha] is the result of Equation 50; BG_Total [tDMha] is the result of Equation 51; and Total_wood [tDMha] is the total (above and below ground) wood storage in tonnes of dry matter per hectare.

3.9 Annual total wood carbon storage was calculated.

Total_wood_{—}C [tCha]=Total_wood [tDMha]*TotalDM_{—}C_ratio Eq. 53

Where Total_wood [tDMha] is the result of Equation 52, TotalDM_C_ratio is the result of Equation 41; and Total_wood_C [tCha] is the total wood carbon storage per hectare.

4.0 Science Plan-Directions 3: Remote sensing processing and statistical analysis of remote sensing imagery prior to modeling vegetation stock attributes with data mining software.

4.1 Science Justification for MODIS imagery and other remote sensing imagery of vegetation:

Above-ground biomass stocks and/or wood carbon storage (i.e., the trunk, branches and other woody elements of a tree) in theory will change very little between years in undisturbed conditions, except by increasing from vegetation growth and/or annual re-growth. Substantial change in biomass stocks, especially decreases in biomass stocks, is due to disturbance and human impact (i.e., deforestation and degradation). After disturbance, green-up ensues in natural regeneration. The IPCC Degradation Report defined forest degradation as: “A direct human-induced long-term loss (persisting for X years or more) of at least Y % of forest carbon stocks [and forest values]since time T and not qualifying as deforestation or an elected activity under Article 3.4 of the Kyoto Protocol (IPCC, 2003b, p. 16).” The IPCC definition for forest degradation requires a regression for annual carbon stocks on the y-axis and time in years along the x-axis. Inter-annual vegetation growth (i.e., NPP) over time can be highly variable and dependent on a number of factors, such as climate, natural disturbance such as grazing by wild herbivores and human impact (i.e., deforestation and forest degradation). However, when assessing inter-annual change for woody biomass stocks (.i.e., biomass and/or carbon storage), the remote sensing measurements between years should have a low percentage (i.e., in the single digits) for coefficient variation when there is no human impact. When variability is high, the vegetation attribute most likely captured by the variability is growth rather than stocks. Therefore, assessing for the least amount of coefficient of variation for remote sensing measurements, prior modeling vegetation stock variables, should be indicative of the best practical remote sensing variable to measure above ground biomass stocks. This also relates to the IPCC definition for forest degradation, because a low percentage for coefficient of variation reverse engineers a regression line between years with little to no change in carbon stocks. This means that the best estimate for inter-annual remote sensing estimates of vegetation woody biomass stocks will be determined by the remote sensing variable with the lowest inter-annual coefficient of variation. Fundamentally, the theoretical approach for monitoring a vegetation attribute found herein is rooted in ecological theory related to assessment of multiple stable states for a target biophysical element over time and space. Applied to remote sensing techniques, each pixel in the remote sensing image represents a unique state (i.e., a quantitative amount) of the target biophysical element (i.e., biomass stocks) at one point in time. The unique state of the biophysical element should be relatively stable over time except for the cycling between disturbance and growth/re-growth. This also means a very small coefficient of variation should be found for the average stable state measurement of the target biophysical element across ecosystems between years when there is no and/or little disturbance. When there is a negative annual trend for the vegetation attribute over time, a disturbance hotspot is indicated by a negative annual trend in the pixel over time. This will also indicate degradation of the target bio-physical element for the monitoring period.

4.2 Justification of MODIS class imagery:

MODIS imagery is used as an example because the raw data is freely available at multiple spatial resolutions and at 8-day temporal replication with long-term planned data continuity. The reason MODIS class imagery is preferred over higher resolution imagery, such as Landsat class imagery, is because MODIS imagery better represents fundamental ecological temporal replication on an annual basis. Alternatively, Landsat style analyses better represents the geography of map-making related to change in land cover with a classified spatial value and temporal replication with global reach on a decadal or bi-decadal increment. Global reach is necessary to develop a standardized product that can be used by everyone. Since the IPCC's definition of forest degradation require annual monitoring of carbon stocks in a regression against time, Landsat class imagery cannot be used to monitor forest degradation on a global basis.

4.3 FIGS. 5D-1 and 5D-2 (FIG. 5D) show an example of the process used to develop Allometric Equations 2. FIGS. 5D-1 and 5D-2 are used with Science Plan-Directions 3 in section 4 in the example of a science plan. FIG. 5D-1 is an example of a flow chart used to develop Allometric Equations 2 for stocks of vegetation attributes and develop allometrics with data mining software. More particularly, FIGS. 5D-1 and 5D-2 provide a schematic used for image processing to determine stocks of vegetation attributes.

4.3.1 In 2202, a variety of incremental (meaning more than 1 imager per year) sensor imagery is used, such as from MODIS sensor imagery at various spatial resolutions, such as at 1 km×1 km, 500 m×500 m and 250 m×250 m resolutions. The primary imagery used is either spectral reflectance in 2204 or a vegetation index in 2206, but any of following products can also be used: 1) MOD/MYD/MCD 15 8-day fraction of Absorbed Photosynthically Active Radiation (fPAR), 2) MOD/MYD/MCD 15 8-day Leaf Area Index (LAI) and 3) MOD/MYD 13 8-day Enhanced Vegetation Index (EVI), and 4) MOD/MYD 13 8-day Normalized Difference Vegetation Index (NOVI), and 5) MOD/MYD 13 8-day Red reflectance, 6) MOD/MYD 13 8-day near-infrared reflectance, 7) MOD/MYD 13 8-day mid-infrared reflectance, MOD/MYD 13 8-day blue reflectance and 8) MCD 43 8-day Nadir BRDF-Adjusted Reflectance (NBAR) for all 7 bands. All MOD MODIS imagery is for the period 2001-present and all MODIS MYD imagery is from about mid-2000.

4.3.2 Images are first processed in 2208 for pixel reliability and quality so as to determine highest quality pixels in the image and to remove low quality pixels (i.e., cloud cover). Raw MODIS imagery has a sub-set with a quality control band(s). For example, MOD/MYD 13 has two quality control sub-sets (band 3 and 12) that are used to assess pixel quality and remove low quality pixels. The gray box in 2210 shows the current knowledge in public domain. Stocks are processed to create a one off composite in 2212. The one off composite is then used in data mining software in 2216 with vegetation attribute data in 2214 to model a one off biomass map in 2218. “One off” meaning a map of stocks that does not have the capability of reproducing stock maps on an incremental basis, such as an annual stock map for multiple years required to fulfill the IPCC definitions of forest degradation and devegetation to determine Y % change over a period of time.

4.3.3 The imagery processed in 2208 (see FIG. 5C) are next processed in 2220 for the following qualitative statists per pixel in the image: 1) annual mean, 2) annual maximum, 3) annual minimum, 4) annual medium, 5) annual standard deviation.

4.3.4 The inter-annual coefficient of variation is used in 2222 to assess for both daily and annual qualitative statistics for the following examples: 1) MOD/MYD/MCD 15 8-day fraction of Absorbed Photosynthically Active Radiation (fPAR), 2) MOD/MYD/MCD 15 8-day Leaf Area Index (LAI) and 3) MOD/MYD 13 8-day Enhanced Vegetation Index (EVI), and 4) MOD/MYD 13 8-day Normalized Difference Vegetation Index (NDVI), and 5) MOD/MYD 13 8-day Red reflectance, 6) MOD/MYD 13 8-day near-infrared reflectance (NIR), 7) MOD/MYD 13 8-day mid-infrared reflectance (MIR), MOD/MYD 13 8-day blue reflectance and 8) MCD 43 8-day Nadir BRDF-Adjusted Reflectance (NBAR) for all 7 bands.

4.3.5 In 2222 (see FIG. 5C), the coefficient of variation is also sampled for either 1) a land cover map showing a minimum of AFOLU defined land cover classes and/or 2) a point file for georeferenced field plots of known biomass and/or a land classification. In 2226, the results for the analysis of average coefficient of variation between 2001-2009 are provided for forest land at MODIS grid tile h19v09 for MOD/MYD 13 8-day, annual mean, annual max and annual min NDVI, EVI, RED, NIR, BLUE and MIR band composites. Note that the 8-day coefficient of variation is highly variable. The least variability is in annual mean and max NDVI and mean EVI. The annual mean and max NDVI and mean EVI will be used in the example of modeling annual biomass stocks with data mining software.

4.4 After 4.3 has been completed to determine the most appropriate band(s) to use to model biomass stocks in 2212, a point file for georeferenced field plot will be overlaid on the bands and sampled for the pixel in which the point falls. The data will then be uploaded into a data-mining software in 2216. The data-mining software is used to generate Allometric Equations 2 in 2228 and annual maps of carbon stocks in 2224.

5.0 Science Plan-Directions 4: Modeling vegetation attributes with a Random Forests model in a data mining software:

5.1 Chapter 5.7 of IPCC GPG-LULUCF (2003a) described the use of remote sensing data and image products as an Approach 4b (p. 5.67) to measure above-ground biomass stored in wood. The chapter stated (p 5.67): “Satellite remote sensing and its image products may also be appropriate for assessing biomass and biomass changes at the major ecosystem level (e.g., grassland vs. forest). Carbon stocks in forests can be estimated using correlations between spectral image data and biomass, provided that adequate data (not used for inventory estimates) are available to represent the range in forest biomes and management regimes for which estimates are required. Correlation equations, may be affected by several parameters (canopy and understorey type, season, illumination, satellite-viewing geometry), and must in general be developed for each forest type. In addition, vegetation indices (e.g., the Normalised Difference Vegetation Index, NDVI) have also been used for the estimation of above ground biomass. The correlation equations referenced above between a measurement from a remote sensing image and a physical measurement of biomass is an allometric equation.

5.2 Justification for MODIS imagery:

Baccini et al (2008) used 7 reflectance bands from MODIS MCD 43 NBAR imagery at 1 km×1 km resolution to develop a Random Forest model with above ground biomass sampled from field observations. The biomass map they developed was a mosaic of best quality observations over a multi-year period. The field observations also came from different years, some outside the period observed with the NBAR imagery, so there may be a temporal mismatch when regressing the field observations with remote sensing imagery. The ultimate result of Baccini et al's work is a one-off biomass map. The issue with this one-off map is that it has no practical application to meeting the annual monitoring and reporting requirements for either the IPCC GPGs or any of the carbon trading mechanisms. This is because annual maps are needed to 1) develop a baseline assessment of a project site and 2) repeat monitoring annual after a project is validated to show that the carbon amount is still there. Hence, the results of Science Plan-Directions 3 are used in Science Plan-Directions 4 to determine which remote sensing variables would be best applied to monitoring inter-annual above ground biomass stocks and their annual change.

5.3 Sampling remote sensing imagery with the point file of field observation coordinates is completed for annual composites of qualitative statistics (annual mean, maximum, minimum, and medium) with the following imagery: 1) MOD/MYD/MCD 15 8-day fraction of Absorbed Photosynthically Active Radiation (fPAR), 2) MOD/MYD/MCD 15 8-day Leaf Area Index (LAI) and 3) MOD/MYD 13 8-day Enhanced Vegetation Index (EVI), and 4) MOD/MYD 13 8-day Normalized Difference Vegetation Index (NDVI), and 5) MOD/MYD 13 8-day Red reflectance, 6) MOD/MYD 13 8-day near-infrared reflectance, 7) MOD/MYD 13 8-day mid-infrared reflectance, MOD/MYD 13 8-day blue reflectance and 8) MCD 43 8-day Nadir BRDF-Adjusted Reflectance (NBAR) for all 7 bands.

5.4 The composites with the lowest coefficient of variation (i.e., at least in single percentages) are used, such as in section 4.3.5.

5.5 A two tiered approach is used to determine which annual composite is used to develop the Random Forests model:

Tier 1: If the field observation(s) are in a year that corresponds directly with the remote sensing composites (i.e., the field observation(s) were sampled in 2009) than the corresponding annual composite(s) from that year is used (i.e., mean annual EVI for 2009).

Tier 2: If the field observation are not in a year that corresponds directly with the remote sensing composites (i.e., the field observation(s) were sampled in 1995), from a period of time that represents a period of multiple years (i.e., one image for the period 2000-2005), and/or unknown, than the mean of the corresponding annual composite(s) is used (i.e., mean annual EVI between 2001-present).

Tier 1 supersedes Tier 2 in this context.

5.6 The data from the remote sensing composite and the field observations are then used as input data for the Random Forest model.

5.7 The Random Forest model is used to model a minimum of 200 decision trees that are in turn used as predictors in the model. 200 decisions trees is normally the smallest suggested amount of decisions trees to grow for the Random Forest model. Any amount of decision trees over 200 can also be run. The model is validated and verified with a percentage of geospatial data withheld from the training model.

5.8 The full remote sensing annual composites are used as input data in the Random Forest model. The decision tree predictors developed in 5.6 are used to score/model vegetation attributes for the full annual remote sensing composites for each temporal replicate in the incremental time series (i.e., for each year).

5.9 Regression analysis is completed for the annual maps of vegetation attribute(s) over time to assess temporal change in the vegetation attribute over space as a map.

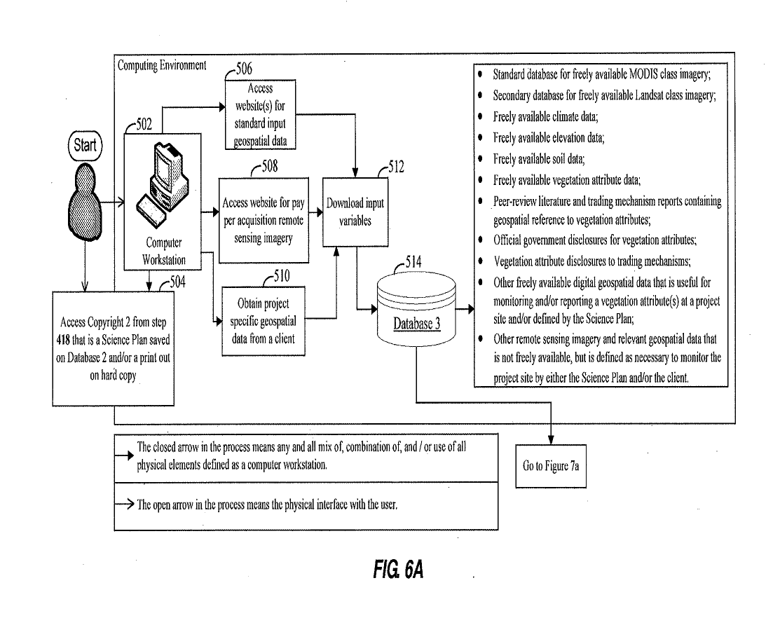

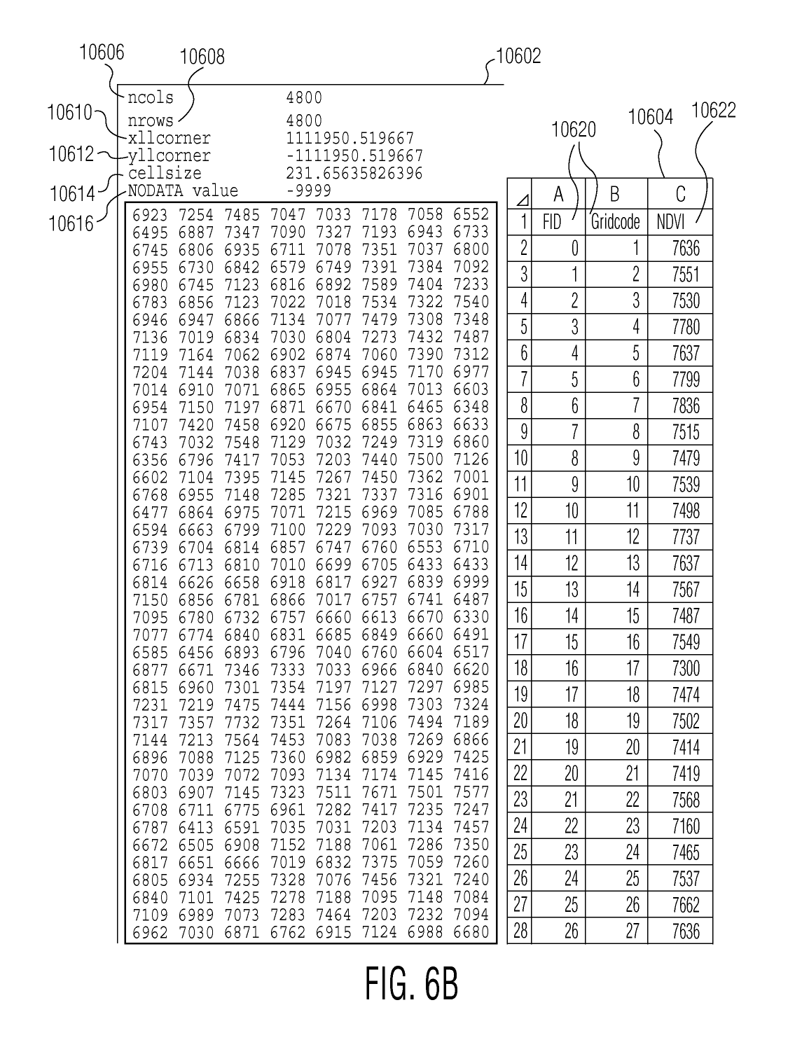

FIG. 6A shows an example for the generic process used to obtain the primary database for input data relevant to the science plan with an example of obtaining the MODerate Resolution Imaging Spectroradiometer (MODIS) remote sensing imagery data referred to in the example of the science plan from 10418. A user accesses a computer workstation in 502 that includes a screen display(s), processor(s), hard drive(s), a keyboard, a mouse, a router connected to the internet and other physical elements related to a computer workstation, etc. A computer workstation 502 in this context can store, retrieve, process and/or output data and/or communicate with other computers. Copyright 2 in 10418 is viewed by the user in 504 as either a printed out hard copy and/or accessed in Portable Document Format (i.e, .pdf and/or similar file format) by a computer workstation in 502. The computer workstation is used to access the websites of relevant standard input geospatial data in 506. Examples of web-sites where MODIS and/or Landsat imagery can be downloaded are the following: <URLs: https://lpdaac.usgs.qov/lpdaac/get_data; https://wist.echo.nasa.gov/wist-bin/api/ims.cgi.?mode=MAINSRGH&JS=1; https:/lpdaac.usgs.gov/lpdaac/get_data/data_pool>. Examples of standard MODIS products that are downloaded and stored on Database 3 include any and/or all of the following: MCD45A1, MOD09GA, MYD09GA, MOD09GQ, MYD09GQ, MOD09CMG, MYD09CMG, MOD09A1, MYD09A1, MOD09Q1, MYD09Q1, MOD13A1, MYD13A1, MOD13A2, MYD13A2, MOD13Q1, MYD13Q1, MOD13A3, MYD13A3, MOD13C1, MYD13C1, MOD13C2, MYD13C2, MOD44W, MOD11_L2, MYD11_L2, MOD11A1, MYD11A1, MOD11A2, MYD11A2, MOD11B1, MYD11B1, MOD11C1, MYD11C1, MOD11C2, MYD11C2, MOD11C3, MYD11C3, MOD14, MYD14, MOD14A1, MYD14A1, MOD14A2, MYD14A2, MCD15A2, MOD15A2, MYD15A2, MOD17A2, MYD17A2, MCD43A3, MCD43B3, MCD43C3, MCD43A1, MCD43B1, MCD43C1, MCD43A2, MCD43B2, MCD43C2, MCD43A4, MCD43B4, MCD43C4, MOD12Q1, MCD12Q1, MOD12Q2, MCD12Q2, MOD12C1, MCD12C1, MOD44B. The websites for freely available climate data are accessed in 506. Examples of freely available climate data (past, present and/or future) that is stored on Database 3 include the World Meteorological Organization's geospatial data, the University of East Angelia Climate Research Unit's publically available gridded climate data, and the NCEP-NCAR publically available gridded climate data. The websites for freely available elevation data are accessed in 506. An example of elevation data that is stored on Database 3 is from the Shuttle Radar Topography Mission (SRTM). The websites for freely available soil data is accessed in 506. An example of soil data that is stored on Database 3 is the ISRIC-WISE revised soil property estimates for soil types of the world. The websites for freely available vegetation attributes is accessed in 506. An example of vegetation attribute data that is stored on Database 3 is FLUXNET data and associated sites. The websites for publishers of peer-review journal articles are accessed in 506. Examples of these website's are ScienceDirect and Wiley InterScience. The websites for the publishers of peer-review journal articles have internal key word search engines. The website key word search engines are used to search for the specific vegetation attribute (i.e., “biomass”) and key word that reference a geographical coordinate (i.e., “latitude” and “longitude”). Any publications related to any input data and/or the use of any input data and/or the use of any computer implemented software to fulfill the science and/or methods disclosed herein are also downloaded. The retrieved files are downloaded and stored on Database 3. The websites of regulated and voluntary trading mechanisms are accessed in 506. Project developers that develop a project site for an offset related to a vegetation attribute must supply documents that report vegetation attributes to the trading mechanism. The reporting documents are normally in the form of Project Design Documents, Project Validation Documents and Project Verification Documents and are publically disclosed on the trading mechanism website. The reporting documents from trading mechanisms are downloaded and stored on Database 3. The downloaded peer-review articles and/or reporting documents from trading mechanisms are then accessed with a text retrieval software and a key word search is performed similar to the process of described in FIG. 4B. The Level 1 key words used in the text retrieval software are for a vegetation attribute (i.e., “biomass”), a numerical amount for all referenced vegetation attributes, the measurement system (i.e., “tC ha”) and the latitude and longitude coordinates. Any additional key words deemed useful by the user are added to a Level 2 key word search. Tables are also retrieved in any of the search levels when the tables are described by a key word. This retrieved information is outputted to a spreadsheet that is stored on Database 3. The Level 1 and Level 2 key words are stored on a meta-database. The aforementioned process is only used on documents when there is no disclaimer limiting the use of the document and/or use of the contents of the document in certain ways described herein. The websites for freely available official governmental disclosures for vegetation attributes are accessed in 506. An example of such a website is the USDA Forest Service Geodata Clearinghouse. Examples of government disclosures for geospatial data of a vegetation attribute stored on Database 3 is Forest Service's biomass maps for the Contiguous US, Alaska and Puerto Rico. Other freely available digital geospatial information that is internet accessible and useful for monitoring and/or reporting vegetation attribute(s) at a project site and/or defined by future science plans are stored on Database 3. In 508, the websites for remote sensing images that are not free are accessed after direction from the client and access to the remote sensing imagery is acquired, downloaded in 512 and stored on Database 3 in 514. Geospatial data for vegetation attributes specifically related to the client's project site are obtained in 510 through an internet interface (i.e., email and/or a website) and downloaded in 512 by the computer workstation and stored on Database 3 in 514. The geospatial data is obtained electronically from the client and/or an agent acting on behalf of the client, either: electronically transmitting, sending, emailing and/or uploading the geospatial data through an internet interface (i.e., email and/or a web-site interface). All geospatial data stored on Database 3 in 514 is stored in standardized raster files (i.e., .hdf, .tif, .img, etc. files) and/or a spreadsheet (i.e., .txt, .dbf, .html, .sql, .csv, etc. files) that can be georeferenced and/or assigned a grid code coordinate and/or as a vector file (i.e., a shapefile with associated files). FIG. 6B shows an example of a georeferenced spreadsheet in 10602 and a spreadsheet with an assigned grid code coordinate in 10604. In 10602, the arrows point to the following: columns in 10606; rows in 10608; x and y corners in 10610 and 10612, respectively; pixel cell size in 10614, no data value in 10616 and the box in 10618 shows the raw data. In 10604, a grid code is pointed to in either of the examples in 10620, where a unique gridded value is assigned to the individual pixel information in 10622 for the whole remote sensing image.

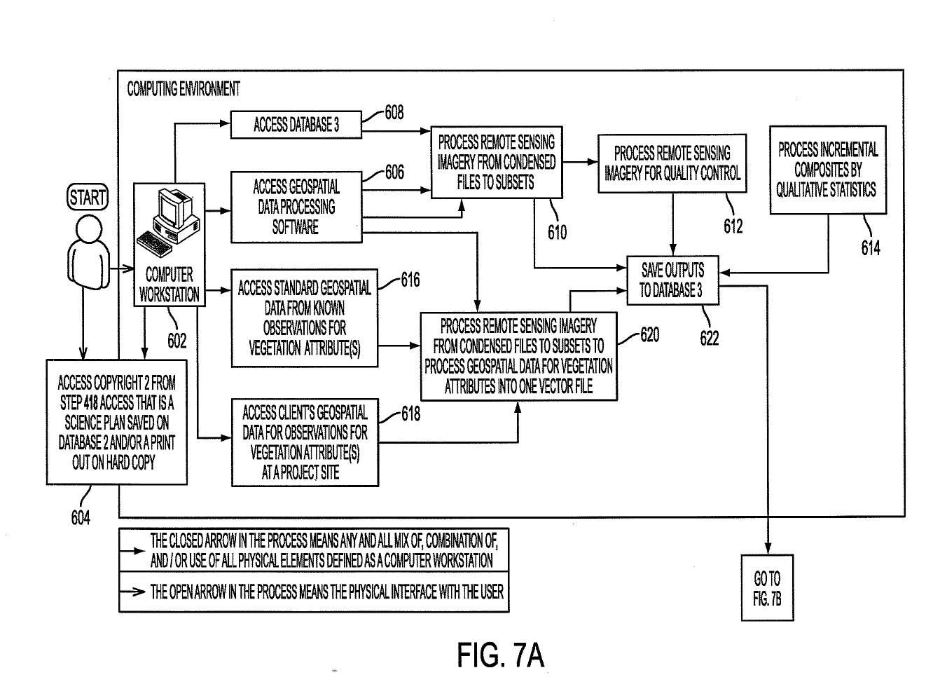

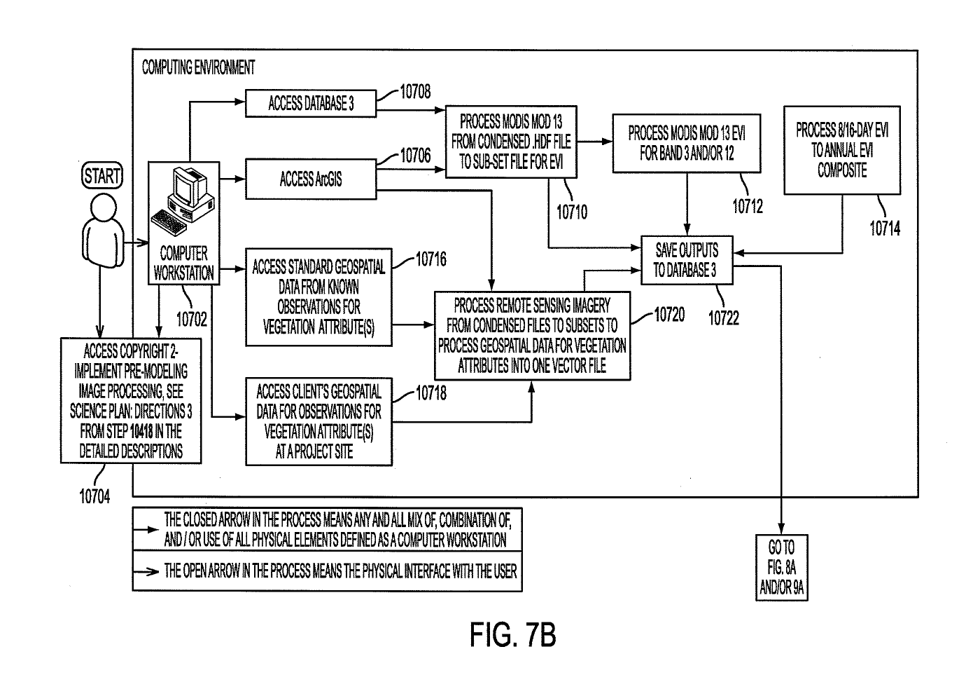

After the standard MODIS products are downloaded, the raw download imagery files are not directly useful for monitoring and/or reporting a project site, because multiple image bands are condensed on one Hierarchical Data Format file (i.e., .hdf file), the individual pixels in an image may be degraded in quality by cloud cover and other data distortions, and the imagery is in 8-day temporal replicates that need to be processed, for example, to an annual temporal replicates for a certain qualitative statistical amount. Hence, the raw downloaded data needs to be preprocessed to prepare the data for use with the directions in the Science Plan. FIG. 7A shows the generic process used to pre-process remote sensing imagery in Database 3. A user accesses a computer workstation in 602 that includes a screen display(s), processor(s), hard drive (s), a keyboard, a mouse, a router connected to the internet and other physical elements related to a computer workstation, etc. A computer workstation 602 in this context can store, retrieve, process and/or output data and/or communicate with other computers. Copyright 2 in 418 is viewed in 604 as either a printed out hard copy and/or accessed in Portable Document Format (i.e, .pdf and/or similar file format) by a computer workstation in 602. The computer workstation is used to access a geospatial data processing software in 606 that has the ability to process geospatial data in line with and fulfill the science plan in 418. Examples of geospatial data processing software are the following: ArcView/GIS, Erdas Imagine, Envi, Idrisi, Quantum GIS, Grass, and Land Change Modeler and/or other relevant image processing software, etc., that is copyrighted and/or copyrightable and/or in open access. The geospatial data processing software is stored on the computer workstation hard drive and installed on the work station. The computer workstation is used with the geospatial data processing software to access Database 3 in 608. The geospatial data processing software is used to sub-set files stored on Database 3 in 610 that are in a condensed file format (i.e., an image file with more than one band per image on the file). After the files are sub-setted, the geospatial data processing software is used in 612 with the Quality Control sub-set to remove low quality and/or contaminated pixels in the imagery. The geospatial data processing software is next used in 614 to develop qualitative statistical composites (i.e., mean, minimum, maximum, medium, standard deviation, etc) for daily, weekly and/or every 8-day, 10-day, 15-day, monthly, and/or annual increments. After the qualitative statistical composites are processed, the different remote sensing imagery and/or bands may be mixed and/or combined to create an index, such as a photochemical reflectance index, a normalized difference water index, a soil adjusted vegetation index, etc. In 616, the standard geospatial data for vegetation attribute(s) is accessed. The standard geospatial data for vegetation attributes can be any data described in Database 3, except data that is obtained under a confidential agreement with a client. In 618, the client's geospatial data for a know vegetation attribute is accessed from Database 3. The client's data is kept separate from the standard data because of confidentially agreements. In 620, any and/or all information for the vegetation attribute is processed with geospatial data processing software to create a point vector file for each at the geographical coordinates of the vegetation attribute(s). The outputs from 610, 612, 614 and 620 are stored on Database 3 in 622 in standardized raster files (i.e., .hdf, .tif, .img, etc. files) and/or a spreadsheet (i.e., .txt, .dbf, .html, .sql, .csv, etc. files) that can be georeferenced and/or assigned a grid code coordinate and/or a geospatial vector file (i.e., a shapefile with associated files). All raster files stored on Database 3 may be processed, merged and/or placed into a mosaic and/or standardized to the WGS-84 geographical coordinate system.

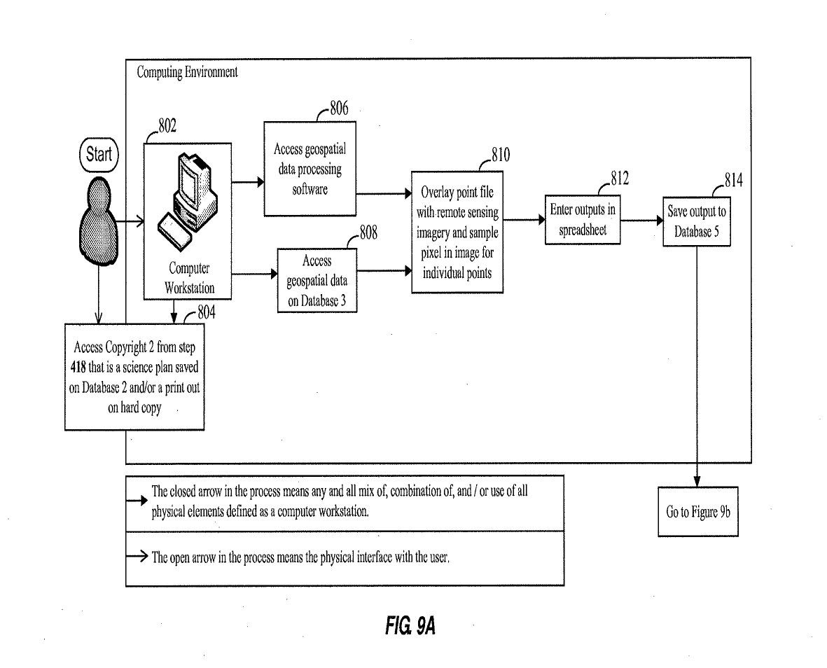

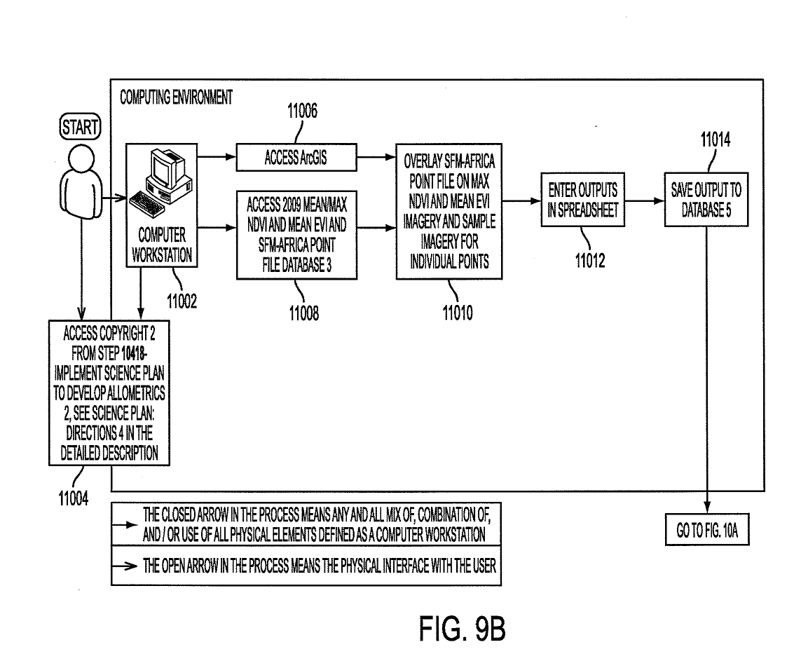

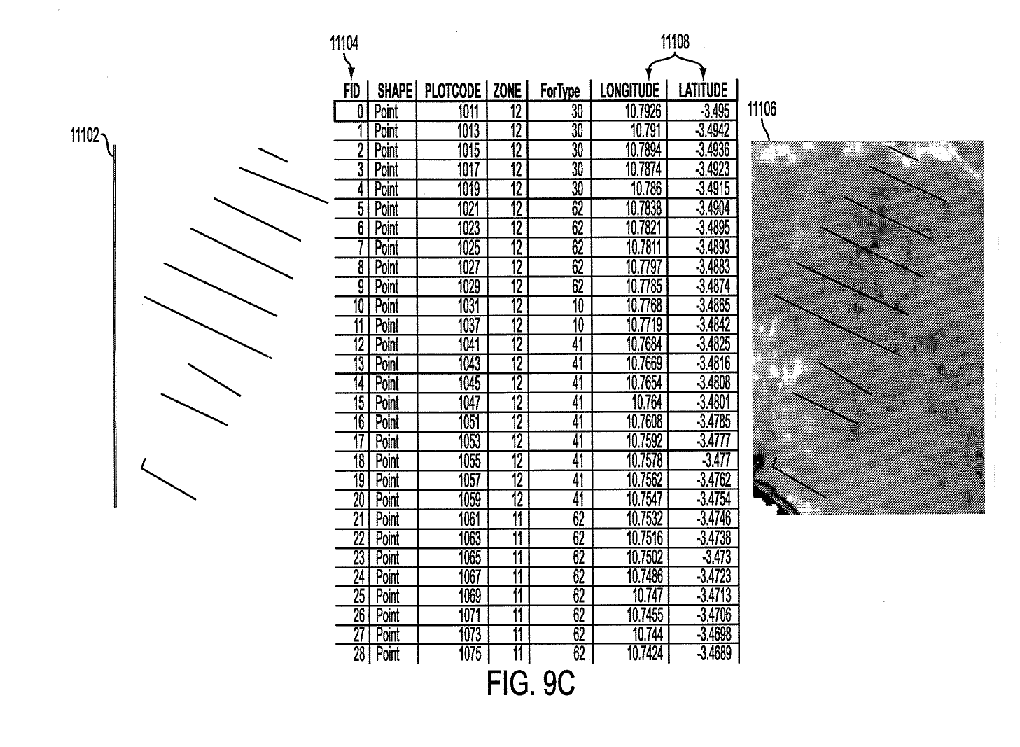

FIG. 7B shows an example for the generic process used to pre-process remote sensing imagery in the primary database applied to MODIS MOD/MYD 13 from FIG. 105. A user accesses a computer workstation in 10702 that includes a screen display(s), processor(s), hard drive(s), a keyboard, a mouse, a router connected to the internet and other physical elements related to a computer workstation, etc. A computer workstation 10702 in this context can store, retrieve, process and/or output data and/or communicate with other computers. Copyright 2 from 10418 is viewed in 10704 as either a printed out hard copy and/or accessed in Portable Document Format (i.e, .pdf and/or similar file format) by a computer workstation 10702. The computer workstation is used to access a geospatial data processing software, ArcGIS in 10706. The computer workstation is used with ArcGIS to access Database 3 in 10708. The geospatial data processing software is used in 10710 to sub-set files stored on Database 3 that are in a condensed file format. For example, MOD 13 is downloaded in a condensed Hierarchical Data Format (i.e., .hdf format). MOD 13 in hdf format stores a variety of sub-sets, including Normalized Difference Vegetation Index (NDVI), Enhanced Vegetation Index (EVI), Red, Blue, Near-Infrared, Mid-Infrared, VI Quality, Pixel Reliability, etc. After the files are processed for sub-sets, ArcGIS is used in 10712 with the Pixel Reliability sub-set and/or VI Quality sub-set to remove low quality and/or contaminated pixels in the image, such as removing pixels with cloud cover from an EVI image. ArcGIS is next used in 10714 to develop qualitative statistical composites (i.e., mean, minimum, maximum, medium, standard deviation, etc) for daily, weekly and/or every 8-day, monthly, and/or annual increments. For example, all 8-day MODIS MOD/MYD EVI image composites from the year 2009 can be processed into one image for the mean annual EVI per pixel in 2009. In 10716, standard geospatial data from known observations stored on Database 3 are accessed, such as the Forest Service's biomass maps in a raster file for the Contiguous US, Alaska and Puerto Rico. These raster files are processed with ArcGIS in 10718 to convert the pixels in the raster file to a point in a vector file. In 10718, a clients geospatial data is accessed from Database 3. The table file with the vegetation attributes is processed in ArcGIS in 10720 to a vector file with a point at the geographical location (i.e., longitude and latitude) for each field observation of a vegetation attribute. An example of the vector file output in 10720 is shown in 11102 from FIG. 9C. This is an example of over 500 field plots obtained from SFM-Africa. Each point in 11102 is indicative of a field observation for a vegetation attribute and a geographical coordinate. In 11104 from FIG. 9C, the arrows in 11108 show the longitude and latitude for each point in 11102. The outputs of 10710, 10712, 10714, and 10720 are stored on Database 3 in 10722 in standardized raster files (i.e., .hdf, .tif, .img, etc. files) and/or a spreadsheet (i.e., .txt, .dbf, .html, .sql, .csv, etc. files) that can be georeferenced and/or assigned a grid code coordinate and/or a geospatial vector file (i.e., a shapefile with associated files). All raster files stored on Database 3 may be processed, merged and/or placed into a mosaic and/or standardized to the WGS-84 geographical coordinate system.

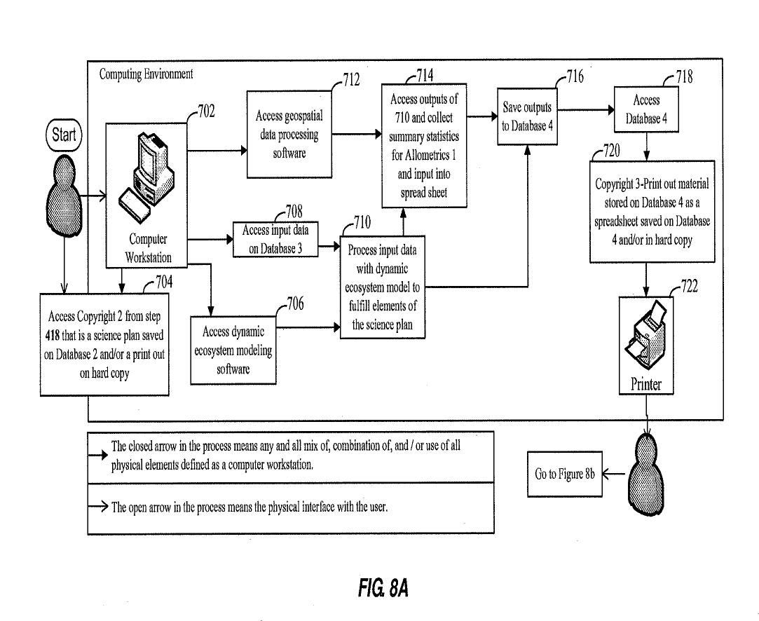

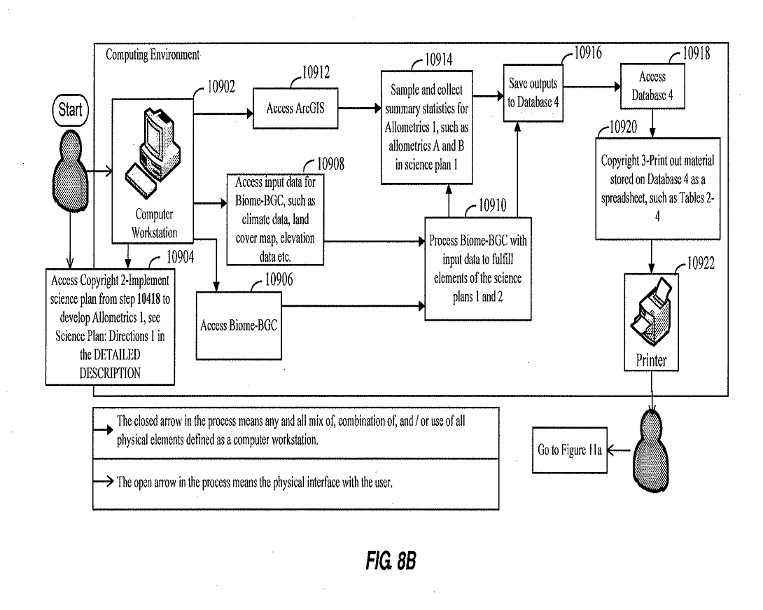

FIG. 8A shows the generic process used to develop Allometric Equations 1. A user accesses a computer workstation in 702 that includes a screen display(s), processor(s), hard drive(s), a keyboard, a mouse, a router connected to the internet and other physical elements related to a computer workstation, etc. A computer workstation 702 in this context can store, retrieve, process and/or output data and/or communicate with other computers. The science plan from 418 is viewed in 704 as either a printed out hard copy and/or accessed in Portable Document Format (i.e., .pdf and/or similar file format) by a computer workstation in 702. The computer workstation is used to access a dynamic ecosystem modeling software in 706. A dynamic ecosystem modeling software means a copyrighted and/or copyrightable software that can be used to quantify GHGs emissions, removals and/or other vegetation attributes defined by the science plan in 418. A dynamic ecosystem model (i.e., a biogeochemical model) may also include any mix and/or combination and/or coupling of a dynamic ecosystem modeling software to one or more other dynamic ecosystem modeling software and/or jointly coupling one or more models to a soil and/or hydraulic model and/or the added coupling to a climate model. The dynamic ecosystem model is stored on the computer workstation hard drive and installed on the work station. The computer workstation is used in 710 to process the dynamic ecosystem model in 706 with input data from 708 stored on Database 3 to fulfill elements of 418. The outputs of the processing in 710 are stored on Database 4 in 716. The computer workstation is next used to access a geospatial data processing software in 712. Examples of geospatial data processing software are the following: ArcView/GIS, Erdas Imagine, Envi, Idrisi, Quantum GIS, Grass, and Land Change Modeler and/or other relevant image processing software, etc., that is copyrighted and/or copyrightable and/or in open access. The image processing software is used to access the outputs of 710 and develop summary statistics that are uploaded into a spreadsheet (i.e., MS Excel) in 714. The summary statistics are used in 714 to reclassify land cover maps for the summary statistics. The outputs of 714 are stored on Database 4 in 716. The outputs from 714, are stored on Database 4 in standardized raster files (i.e., .hdf, .tif, .img, etc. files) and/or a spreadsheet/table and/or a spreadsheet (i.e., .txt, .dbf, .html, .sql, .csv, etc. files) that can be georeferenced and/or assigned a grid code coordinate and/or a geospatial vector file (i.e., a shapefile with associated files). The material stored on 716 is accessed in 718 with spreadsheet software (i.e. MS Excel), printed out as a spreadsheet and/or a table and/or an atlas/illustration and defined as Copyright 3 in 720 in either a Portable Document Format (i.e, .pdf and/or similar file format) digital file and/or in hard copy with a printer in 722.

FIG. 8B shows an example for the generic process used to develop Allometric Equations 1 applied to dynamic ecosystem model Biome-BGC. A user accesses a computer workstation in 10902 that includes a screen display(s), processor(s), hard drive(s), a keyboard, a mouse, a router connected to the internet and other physical elements related to a computer workstation, etc. A computer workstation 10902 in this context can store, retrieve, process and/or output data and/or communicate with other computers. The elements of Science Plan-Directions 1 in FIG. 8B from 10418 are viewed in 10904 as either a printed out hard copy and/or accessed in Portable Document Format (i.e, .pdf and/or similar file format) by a computer workstation in 10902. The computer workstation is used to access Biome-BGC in 10906. Biome-BGC may also be mixed and/or combined and/or coupled with to one or more other dynamic ecosystem modeling software and/or jointly coupled to a soil and/or hydraulic model (such as Century) and/or coupled to a climate model (such as ANU-Spline). The computer workstation is used in 10910 to process the Biome-BGC in 10906 with input data from 10908 stored on Database 3 (such as various climate data, soil data, elevation data, etc.) to fulfill elements of Science Plan-Directions 1 in FIG. 8B from 10418. The outputs of the processing in 10910 are stored on Database 4 in 10916. The computer workstation is next used to access ArcGIS in 10912. ArcGIS is used to access the outputs of 10910 and develop summary statistics for land cover that are uploaded into a spreadsheet (i.e., MS Excel) in 10914. The summary statistics are used in 10914 to reclassify land cover maps for the summary statistics. The outputs of 10914 are stored on Database 4 in 10916. The outputs from 10914, are stored on Database 4 in standardized raster files (i.e., .hdf, .tif, .img, etc. files) and/or a spreadsheet/table and/or a spreadsheet (i.e., .txt, .dbf, .html, .sql, .csv, etc. files) that can be georeferenced and/or assigned a grid code coordinate and/or a geospatial vector file (i.e., a shapefile with associated files). The material stored on 10916 is accessed in 10918 with spreadsheet software (i.e. MS Excel), printed out as a spreadsheet and/or a table and/or an atlas/illustration and defined as Copyright 3 in 10920 in either a Portable Document Format (i.e, .pdf and/or similar file format) digital file and/or in hard copy with a printer in 10922. Table 2 defines Tables 3 and 4. Tables 3 and 4 are examples of outputs from 10922.

TABLE 2

| Parameter | Units | Description |
|---|---|---|
| χ̅ΔC_{NPP} | (g C m⁻²) | Mean Net Primary Production |
| χ̅ΔC_{LF} | (g C m⁻²) | Mean partitioning to leaf carbon |
| χ̅ΔC_{ST} | (g C m⁻²) | Mean partitioning to stem carbon |
| AB₁ _ LF _ ratio | None | Ratio of leaf carbon to net primary |
| | | production |
| AB₂ _ ST _ ratio | None | Ratio of stem carbon to net primary |
| | | production |
| χ̅ΔC_{FR} | (g C m⁻²) | Mean partitioning to fine root carbon |
| χ̅ΔC_{CR} | (g C m⁻²) | Mean partitioning to coarse root |
| | | carbon |
| BB₁ _ FR _ ratio | None | Ratio of fine root carbon to net |
| | | primary production |
| BB₂ _ CR _ ratio | None | Ratio of coarse root carbon to net |
| | | primary production |
| χ̅ΔC_{DW} | (g C m⁻²) | Mean partitioning to dead wood |
| | | debris carbon |
| DW₁ _ ST _ ratio | None | Ratio of dead wood debris carbon |
| | | to stem carbon |
| DW₂ _ CR _ ratio | None | Ratio of dead wood debris carbon |
| | | to coarse root carbon |
| χ̅ΔC_{LI} | (g C m⁻²) | Mean partitioning to litter carbon |
| LI₁ _ LF _ ratio | None | Ratio of litter carbon to leaf carbon |
| LI₂ _ ST _ ratio | None | Ratio of litter carbon to stem carbon |
| LI₃ _ FR _ ratio | None | Ratio of fine litter carbon to fine root |
| | | carbon |
| LI₄ _ CR _ ratio | None | Ratio of litter carbon to coarse root |
| | | carbon |
| LI₅ _ DW _ ratio | None | Ratio of litter carbon to dead wood |
| | | debris carbon |
| χ̅ΔC_{SO} | (g C m⁻²) | Mean partitioning to soil organic |
| | | matter carbon |
| SO₁ _ LI _ ratio | None | Ratio of soil organic matter carbon |
| | | to litter carbon |
| χ̅ΔC_{HR} | (g C m⁻²) | Mean release to heterotrophic respira- |
| | | tion carbon |
| HR₁ _ LI _ ratio | None | Ratio of heterotrophic respiration |
| | | carbon to litter carbon |
| HR₁ _ SO _ ratio | None | Ration of heterotrophic respiration |
| | | carbon to soil organic matter carbon |

TABLE 3

| | Biome Classification | | | | | |
|---|---|---|---|---|---|---|
| Parameter | ENF | EBF | DNF | DBF | MF | Cshrub |
| χ̅ΔC_{NPP} (g Cm⁻²) | 470.51 | 1083.45 | 312.54 | 592.49 | 528.38 | 426.72 |
| χ̅ΔC_{LF} (g Cm⁻²) | 96.79 | 328.23 | 66.41 | 125.90 | 119.78 | 187.84 |
| χ̅ΔC_{ST} (g Cm⁻²) | 212.94 | 328.23 | 146.10 | 276.97 | 223.69 | 41.32 |
| AB_{1—}LF_ratio (g Cm⁻²/g Cm⁻²) | 0.21 | 0.30 | 0.21 | 0.21 | 0.23 | 0.44 |
| AB_{2—}ST_ratio (g Cm⁻²/g Cm⁻²) | 0.45 | 0.30 | 0.47 | 0.47 | 0.42 | 0.10 |
| χ̅ΔC_{FR} (g Cm⁻²) | 96.79 | 328.23 | 66.41 | 125.90 | 122.83 | 187.84 |
| χ̅ΔC_{CR} (g Cm⁻²) | 63.88 | 98.47 | 33.60 | 63.70 | 61.98 | 12.40 |
| BB_{1—}FR_ratio (g Cm⁻²/g Cm⁻²) | 0.21 | 0.30 | 0.21 | 0.21 | 0.23 | 0.44 |
| BB_{2—}CR_ratio (g Cm⁻²/g Cm⁻²) | 0.13 | 0.09 | 0.11 | 0.11 | 0.12 | 0.03 |
| Corresponding UMD Land Cover | 1 | 2 | 3 | 4 | 5 | 8 |
| Classification | | | | | | |

| | Biome Classification | | | | |
|---|---|---|---|---|---|
| Parameter | Oshrub | WL | Wgrass | Grass | Crop |
| χ̅ΔC_{NPP} (g Cm⁻²) | 196.77 | 661.65 | 628.77 | 265.25 | 412.61 |
| χ̅ΔC_{LF} (g Cm⁻²) | 86.32 | 205.89 | 210.11 | 88.41 | 137.53 |
| χ̅ΔC_{ST} (g Cm⁻²) | 18.99 | 133.57 | 45.93 | 0.00 | 0.00 |
| AB_{1—}LF_ratio (g Cm⁻²/g Cm⁻²) | 0.44 | 0.31 | 0.33 | 0.33 | 0.33 |
| AB_{2—}ST_ratio (g Cm⁻²/g Cm⁻²) | 0.10 | 0.20 | 0.07 | 0.00 | 0.00 |
| χ̅ΔC_{FR} (g Cm⁻²) | 86.32 | 285.35 | 360.75 | 176.81 | 275.05 |
| χ̅ΔC_{CR} (g Cm⁻²) | 5.70 | 37.06 | 12.30 | 0.00 | 0.00 |
| BB_{1—}FR_ratio (g Cm⁻²/g Cm⁻²) | 0.44 | 0.43 | 0.57 | 0.67 | 0.67 |
| BB_{2—}CR_ratio (g Cm⁻²/g Cm⁻²) | 0.03 | 0.06 | 0.02 | 0.00 | 0.00 |
| Corresponding UMD Land Cover | 9 | 6 | 7 | 10 | 12 |
| Classification | | | | | |

TABLE 4

| | Biome Classification | | | | | |
|---|---|---|---|---|---|---|
| Parameter | ENF | EBF | DNF | DBF | MF | Cshrub |
| χ̅ΔC_{DW} (g Cm⁻²) | 202.74 | 310.48 | 136.65 | 263.84 | 213.48 | 36.67 |
| DW₁_ST_ratio (g Cm⁻²/g Cm⁻²) | 0.95 | 0.95 | 0.94 | 0.95 | 0.95 | 0.89 |
| DW₂_CR_ratio (g Cm⁻²/g Cm⁻²) | 0.95 | 0.95 | 0.94 | 0.95 | 0.95 | 0.89 |
| χ̅ΔC_{LI} (g Cm⁻²) | 438.73 | 1010.88 | 284.84 | 554.01 | 493.95 | 384.12 |
| LI_{1—}LF_ratio (g Cm⁻²/g Cm⁻²) | 0.96 | 0.95 | 0.97 | 0.97 | 0.96 | 0.90 |
| LI_{2—}ST_ratio (g Cm⁻²/g Cm⁻²) | 0.003 | 0.002 | 0.002 | 0.002 | 0.002 | 0.009 |
| BGC_LI₃_FR_ratio | 0.96 | 0.95 | 0.97 | 0.97 | 0.96 | 0.90 |
| (g Cm⁻²/g Cm⁻²) | | | | | | |
| LI_{4—}CR_ratio (g Cm⁻²/g Cm⁻²) | 0.003 | 0.002 | 0.003 | 0.002 | 0.002 | 0.01 |
| LI_{5—}DW_ratio(g Cm⁻²/g Cm⁻²) | 0.96 | 0.96 | 0.93 | 0.95 | 0.95 | 0.94 |
| χ̅ΔC_{SO} (g Cm⁻²) | 229.65 | 537.36 | 145.98 | 289.07 | 258.46 | 206.14 |
| SO_{1—}LI_ratio (g Cm⁻²/g Cm⁻²) | 0.52 | 0.53 | 0.51 | 0.52 | 0.52 | 0.54 |
| χ̅ΔC_{HR} (g Cm⁻²) | 199.94 | 450.27 | 127.54 | 252.91 | 223.52 | 163.55 |
| HR_{1—}LI_ratio (g Cm⁻²/g Cm⁻²) | 0.46 | 0.45 | 0.45 | 0.46 | 0.45 | 0.43 |
| HR_{1—}SO_ratio (g Cm⁻²/g Cm⁻²) | 1.0 | 1.0 | 1.0 | 1.0 | 1.0 | 0.99 |
| Corresponding UMD Land Cover Class | 1 | 2 | 3 | 4 | 5 | 8 |

| | Biome Classification | | | | |
|---|---|---|---|---|---|
| Parameter | Oshrub | WL | Wgrass | Grass | Crop |
| χ̅ΔC_{DW} (g Cm⁻²) | 17.18 | 125.71 | 42.78 | 0.00 | 0.00 |
| DW_{1—}ST_ratio (g Cm⁻²/g Cm⁻²) | 0.90 | 0.94 | 0.93 | n/a | n/a |
| DW_{2—}CR_ratio (g Cm⁻²/g Cm⁻²) | 0.90 | 0.94 | 0.93 | n/a | n/a |
| χ̅ΔC_{LI} (g Cm⁻²) | 179.98 | 625.37 | 605.09 | 259.32 | 408.24 |
| LI_{1—}LF_ratio (kg Cm⁻²/g Cm⁻²) | 0.92 | 0.96 | 0.97 | 0.98 | 0.99 |
| LI_{2—}ST_ratio (g Cm⁻²/g Cm⁻²) | 0.009 | 0.003 | 0.003 | n/a | n/a |
| LI₃_FR_ratio (g Cm⁻²/g Cm⁻²) | 0.92 | 0.97 | 0.97 | 0.98 | 0.99 |
| LI_{4—}CR_ratio (g Cm⁻²/g Cm⁻²) | 0.009 | 0.003 | 0.003 | n/a | n/a |
| LI_{5—}DW_ratio(g Cm⁻²/g Cm⁻²) | 0.95 | 0.95 | 0.95 | n/a | n/a |
| χ̅ΔC_{SO} (g Cm⁻²) | 96.44 | 335.38 | 327.73 | 140.63 | 224.69 |
| SO_{1—}LI_ratio (g Cm⁻²/g Cm⁻²) | 0.54 | 0.54 | 0.54 | 0.54 | 0.55 |
| χ̅ΔC_{HR} (g Cm⁻²) | 76.58 | 274.95 | 261.24 | 110.61 | 176.52 |
| HR_{1—}LI_ratio (g Cm⁻²/g Cm⁻²) | 0.43 | 0.44 | 0.43 | 0.43 | 0.43 |
| HR_{1—}SO_ratio (g Cm⁻²/g Cm⁻²) | 1.0 | 1.0 | 1.0 | 1.0 | 1.0 |
| Corresponding UMD Land Cover Class | 9 | 6 | 7 | 10 | 12 |

FIG. 9A shows the first half of the generic process used to develop Allometric Equations 2. A user accesses a computer workstation in 802 that includes a screen display(s), processor(s), hard drive(s), a keyboard, a mouse, a router connected to the internet and other physical elements related to a computer workstation, etc. A computer workstation 802 in this context can store, retrieve, process and/or output data and/or communicate with other computers. Copyright 2 in 418 is viewed in 804 as either a printed out hard copy and/or accessed in Portable Document Format (i.e., .pdf and/or similar file format) by a computer workstation 802. The computer workstation is used to access geospatial data processing software in 806. In 808, Database 3 is accessed by the computer workstation for the geospatial data pertaining to 1) remote sensing imagery and 2) vegetation attributes in a vector file. In 810, the geospatial data processing software is used to overlay the vegetation attribute vector file on the remote sensing imagery. The geospatial data processing software is then used to sample the remote sensing imagery per point in the vegetation attribute vector file. The outputs of 810 are entered into a spreadsheet (i.e., MS Excel) in 812 and stored on Database 5 in 814.

FIG. 9B shows the first half of the generic process used to develop Allometric Equations 2 applied with Science Plan-Directions 4 from 10418. A user accesses a computer workstation in 1102 that includes a screen display(s), processor(s), hard drive(s), a keyboard, a mouse, a router connected to the internet and other physical elements related to a computer workstation, etc. A computer workstation 11002 in this context can store, retrieve, process and/or output data and/or communicate with other computers. Copyright 2 in 10418 is viewed in 11004 as either a printed out hard copy and/or accessed in Portable Document Format (i.e., .pdf and/or similar file format) by a computer workstation in 11002. The computer workstation is used to access ArcGIS in 11006. In 11008, the computer workstation is used to access Database 3 for geospatial data 1) for composites of 2009 MODIS mean/max NOVI and mean EVI at 250 m×250 m spatial resolution in 2009 and 2) a vector file containing 551 points of geospatial data for vegetation attribute obtained in 2009. The decision to use 2009 mean/max NOVI and mean EVI was determined by Science Plan-Directions 3 and the results exemplified for coefficient of variation in 2226 from FIG. 5D-2. In this instance, the geospatial data for the 551 points in the vector file was obtained from SFM-Africa. The vegetation attributes were collected by SFM-Africa from field observation in 2009 for basal area, which SFM-Africa then used with allometric equations to calculate volume and above-ground biomass per point. In 11010, ArcGIS is used to overlay the 551 points in the vector file on the 2009 MODIS mean/max NOVI and mean EVI at 250 m×250 m spatial resolution. In 11106 from FIG. 9C, the image shows a visual representation of the 551 field plots overlaid on 2009 mean EVI at 250 m×250 m resolution. ArcGIS is then used to sample the 2009 MODIS mean/max NDVI and mean EVI at 250 m×250 m spatial resolution per point in the vector file containing 551 points of geospatial data. The outputs of 11010 are entered into a spreadsheet (i.e., MS Excel) in 11012 and stored on Database 5 in 11014.

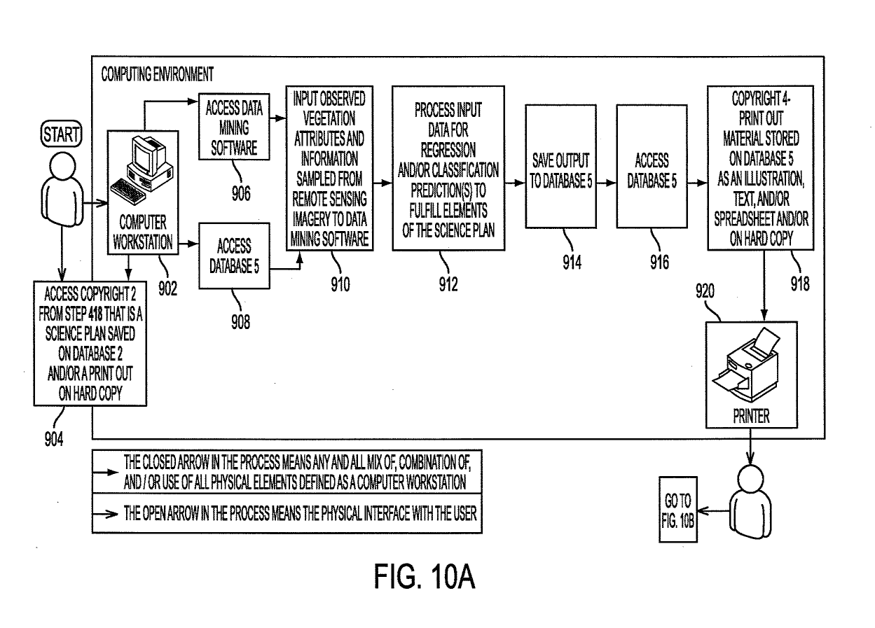

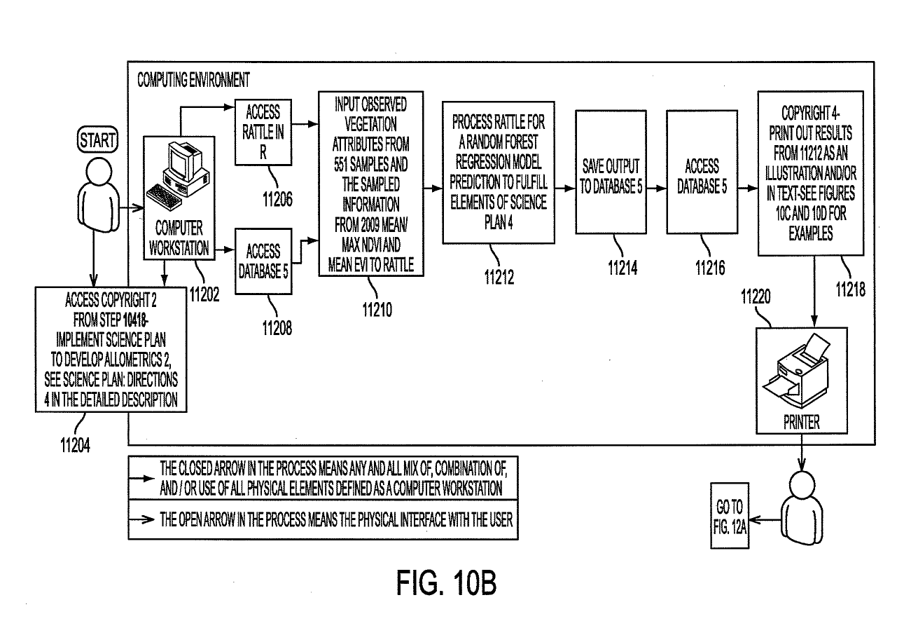

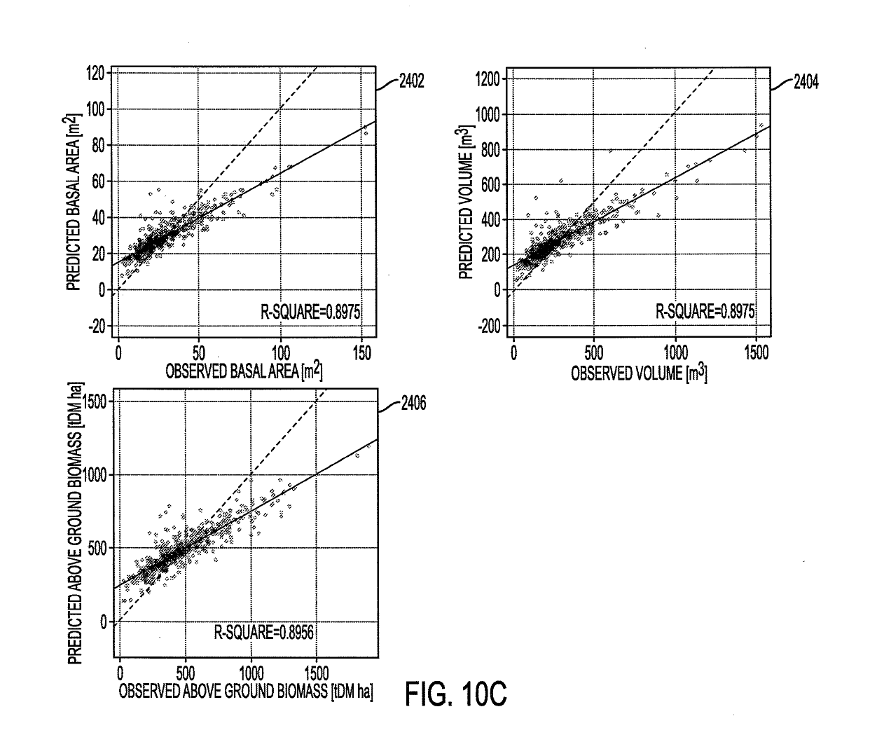

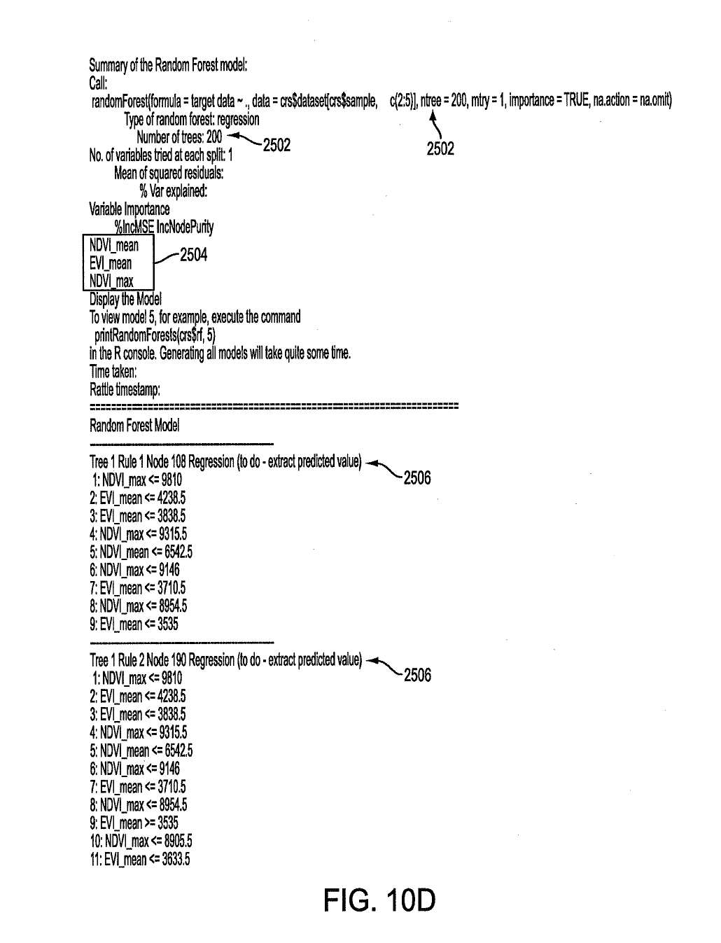

FIG. 10A shows the second half of the process used to develop Allometric Equations 2. A user accesses a computer workstation in 902 that includes a screen display(s), processor(s), hard drive (s), a keyboard, a mouse, a router connected to the internet and other physical elements related to a computer workstation, etc.). A computer workstation 902 in this context can store, retrieve, process and/or output data and/or communicate with other computers. Copyright 2 in 418 is viewed in 904 as either a printed out hard copy and/or accessed in Portable Document Format (i.e, .pdf and/or similar file document format) by a computer workstation. The computer work station is used to access data mining software in 906. Data mining software in this context means a software package (i.e., advanced statistical operations developed by the field of machine learning) that can be used to train a predictive regression and/or classification model between remote sensing imagery of vegetation (i.e., input data) and field observations of vegetation attributes (i.e., target data). Examples of data mining software are Orange, Weka, Rattle and/or any application, variation and/or version of the software R. The spreadsheets from 812 stored on Database 5 are accessed in 908 and entered as input data into the data mining software in 910. In 912, the data mining software is processed with the input data to develop a training dataset for a predictive model based on observed vegetation attributes from the predictor input data. The outputs of 912 are stored on Database 5 in 914. The outputs of 912 are accessed in 916, printed out as a spreadsheet and/or a graphical illustration and defined as Copyright 4 in 918 in either a Portable Document Format (i.e., .pdf and/or similar file document format) digital file and/or in hard copy with a printer in 920.

FIG. 10B shows an example for the second half of the generic process used to develop Allometric Equations 2 applied to the data mining software Rattle in R. A user accesses a computer workstation in 11202 that includes a screen display(s), processor(s), hard drive(s), a keyboard, a mouse, a router connected to the internet and other physical elements related to a computer workstation, etc. A computer workstation 11202 in this context can store, retrieve, process and/or output data and/or communicate with other computers. Copyright 2 in 10418 is viewed in 11204 as either a printed out hard copy and/or accessed in Portable Document Format (i.e, .pdf and/or similar file document format) by a computer workstation 11202. The computer workstation is used to access Rattle in R in 11206. The spreadsheet containing the samples for 2009 MODIS mean/max NDVI and mean EVI and SFM-Africa's 551 field observations from 11012 that are stored on Database 5 are accessed in 11208 and entered into Rattle in 11210. In 11212, Rattle is processed with the said spreadsheet from 11208 to develop a training model with a Random Forest regression predictive function (see Science Plan-Directions 4 in 10418). The outputs of the training model from 11212 are stored on Database 5 in 11214. The outputs of the training model are 11212 are accessed in 11216, printed out as a spreadsheet, text and/or a graphical illustration and defined as Copyright 4 in 11218 in either a Portable Document Format (i.e., .pdf and/or similar file format) digital file and/or in hard copy with a printer in 11220. FIG. 10C shows an example of an illustration for results of the Random Forest training model for the following: basal area in 2402, volume in 2404 and above-ground biomass in 2406. FIG. 10D shows the text output for results of a Random Forest training model for basal area from 2402. The number of decision trees grown was 200, which is the minimum suggested decision trees to grow for a Random Forest model and this is shown in 2502. The mean/max NDVI and mean EVI inputs are shown in 2504. Examples of decision tree rules for the training model are shown in 2506.

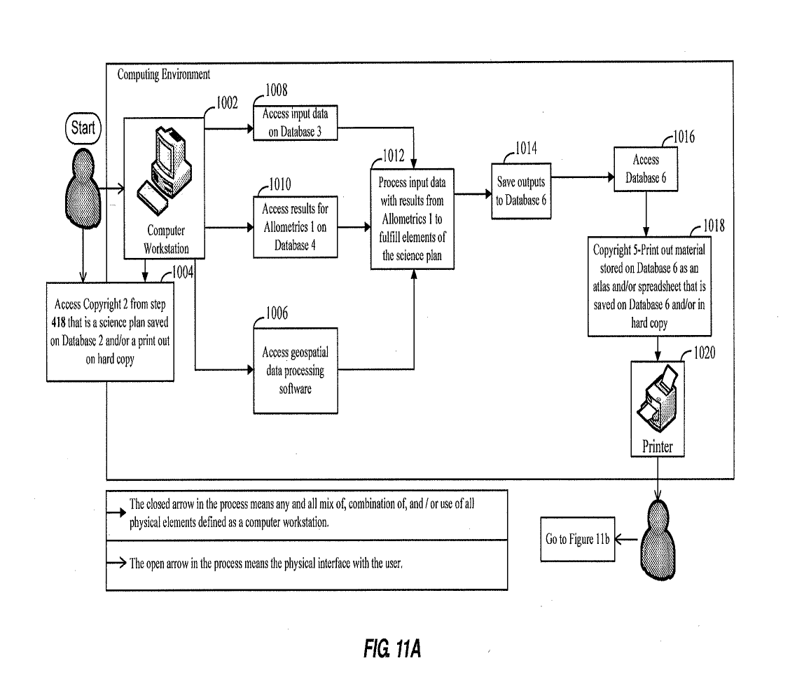

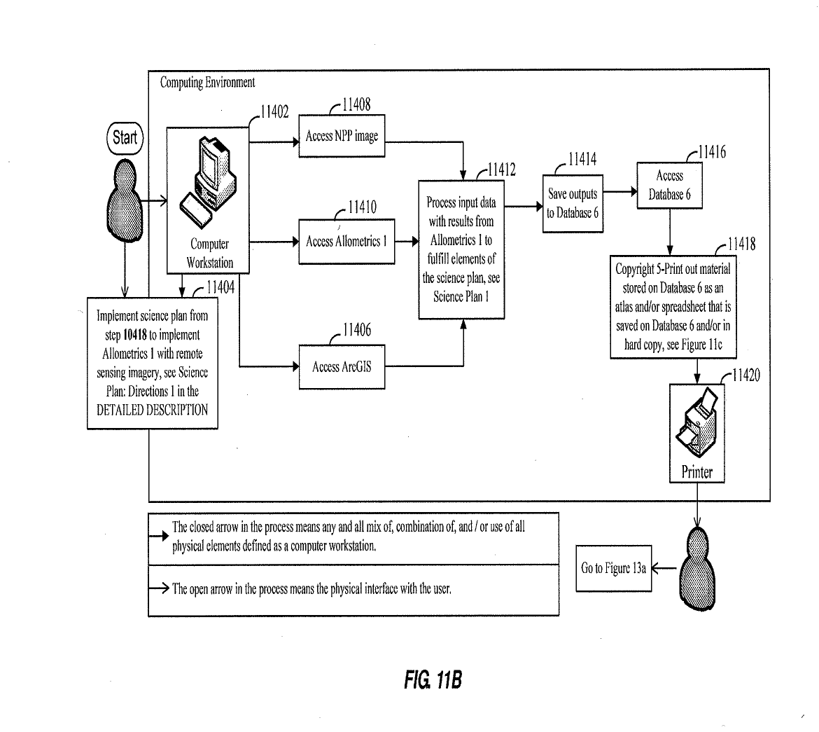

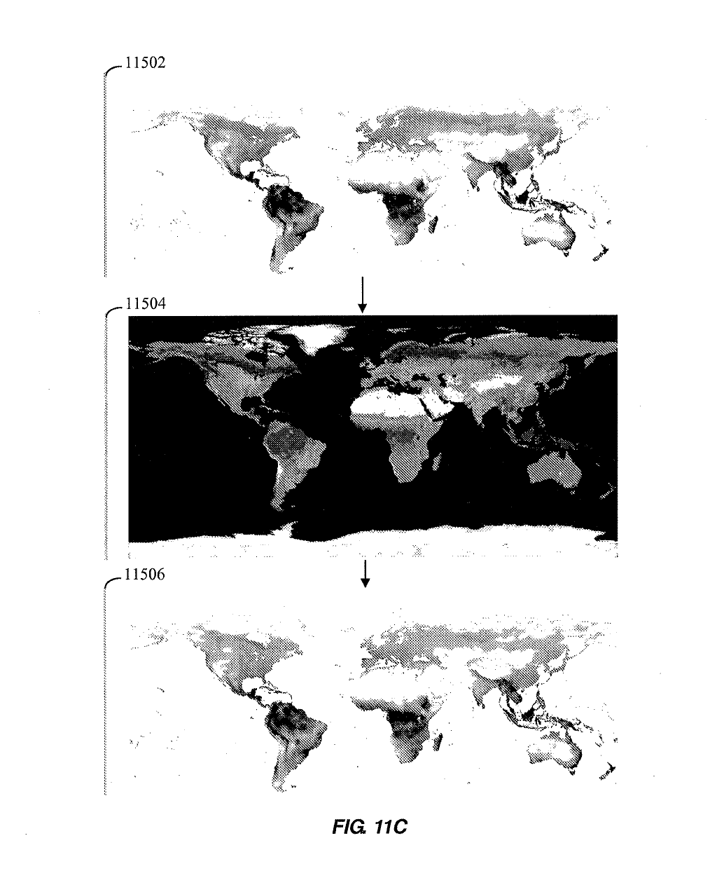

FIG. 11A shows the generic process for implementing Allometric Equations 1 with input data. A user accesses a computer workstation in 1002 that includes a screen display(s), processor(s), hard drive(s), a keyboard, a mouse, a router connected to the internet and other physical elements related to a computer workstation, etc. A computer workstation 1002 in this context can store, retrieve, process and/or output data and/or communicate with other computers. Copyright 2 from 418 is viewed in 1004 as either a printed out hard copy and/or accessed in Portable Document Format (i.e., .pdf and/or similar file format) by a computer workstation 1002. The computer workstation is used to access a geospatial data processing software in 1006. Examples of geospatial data processing software are the following: ArcView/GIS, Erdas Imagine, Envi, Idrisi, Quantum GIS, Grass, and Land Change Modeler and/or other relevant image processing software, etc., that is copyrighted and/or copyrightable and/or in open access. The input data stored on Database 3 is accessed with the geospatial data processing software in 1008. The results for Copyright 3 in 720 from the outputs for the development of Allometric Equations 1 are accessed on Database 4 in 1010. In 1012, the geospatial data processing software is used to process the input data from 1008 with Allometric Equations 1 in 1010 to fulfill the final elements for Allometric Equations 1 in the science plan from 418. The outputs of 1012 are stored on Database 6 in 1014. The outputs of 1012 are accessed in 1016, printed out as a spreadsheet and/or a graphical illustration and defined as Copyright 5 in 1018 as either a Portable Document Format (i.e, .pdf and/or similar file document format) digital file and/or in hard copy with a printer 1020.

FIG. 11B shows an example for the generic process for implementing Allometric Equations 1 with input data applied to the allometrics developed in FIG. 8B and Science Plan-Directions 1. A user accesses a computer workstation in 11402 that includes a screen display(s), processor(s), hard drive(s), a keyboard, a mouse, a router connected to the internet and other physical elements related to a computer workstation, etc. A computer workstation 11402 in this context can store, retrieve, process and/or output data and/or communicate with other computers. Copyright 2 from 10418 is viewed in 11404 as either a printed out hard copy and/or accessed in Portable Document Format (i.e, .pdf and/or similar file format) by a computer workstation 11402. The computer workstation is used to access ArcGIS in 11406. The input data for Net Primary Production (NPP) stored on Database 3 are accessed with ArcGIS in 11408. The results for Allometric Equations 1 from 10920 stored on Database 5 as Copyright 3 are accessed with ArcGIS in 11410. In 11412, ArcGIS is used to process to process NPP in 11408 with the results for Allometric Equations 1 in 11410 to fulfill the final elements of implementing Allometics 1 in the Science Plan-Directions 1 from 10418. The outputs of 11412 are stored on Database 6 in 11414. The outputs of 11412 are accessed in 11416, printed out as a spreadsheet and/or a graphical illustration and defined as Copyright 5 in 11418 as either a Portable Document Format (i.e, .pdf and/or similar file format) digital file and/or in hard copy with a printer 11420. FIG. 11c shows an illustrative example of implementing Allometric Equations 1 with Net Primary Production in 11502. In 11504, the Allometric Equations for the NPP to leaf carbon ratio (reported in Table 3 as AB_{1—}LF_ratio) are shown as a raster map. Equation 17 from 10418 was used to calculate leaf carbon with NPP in 11502 and the NPP to leaf carbon ratio in 11504. The output for Equation 17 is shown in 11506.

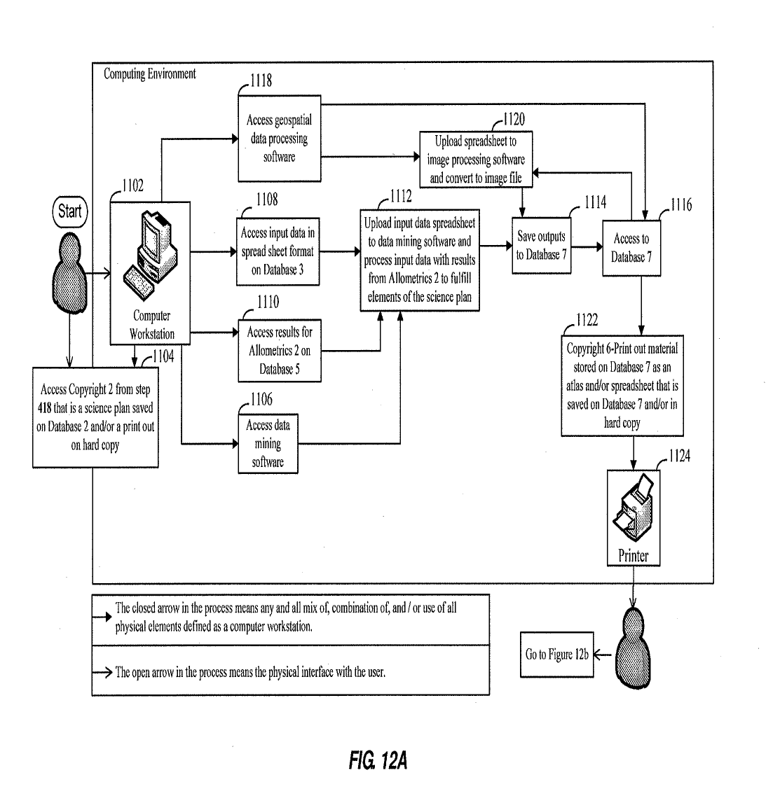

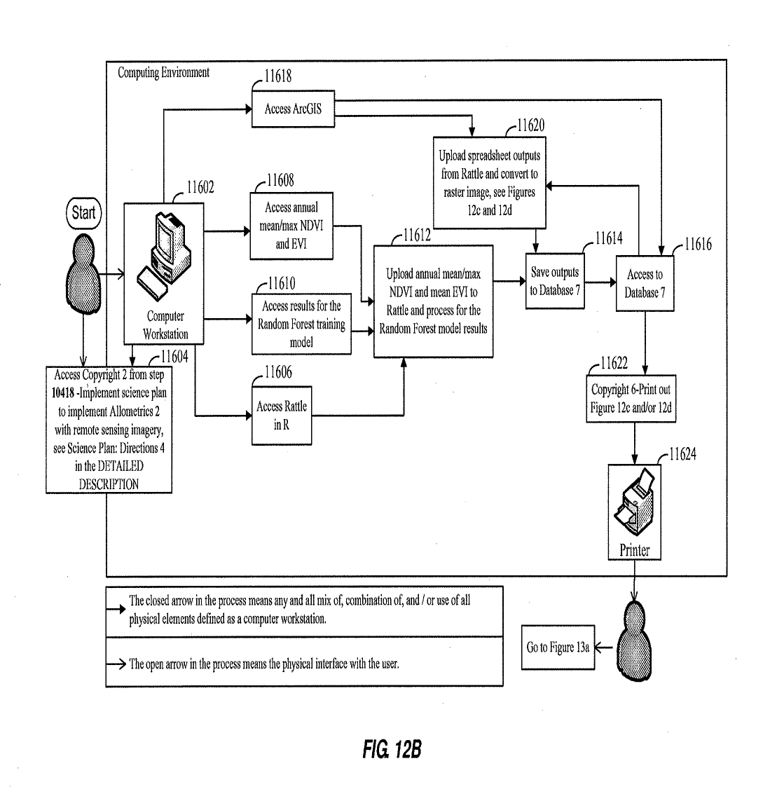

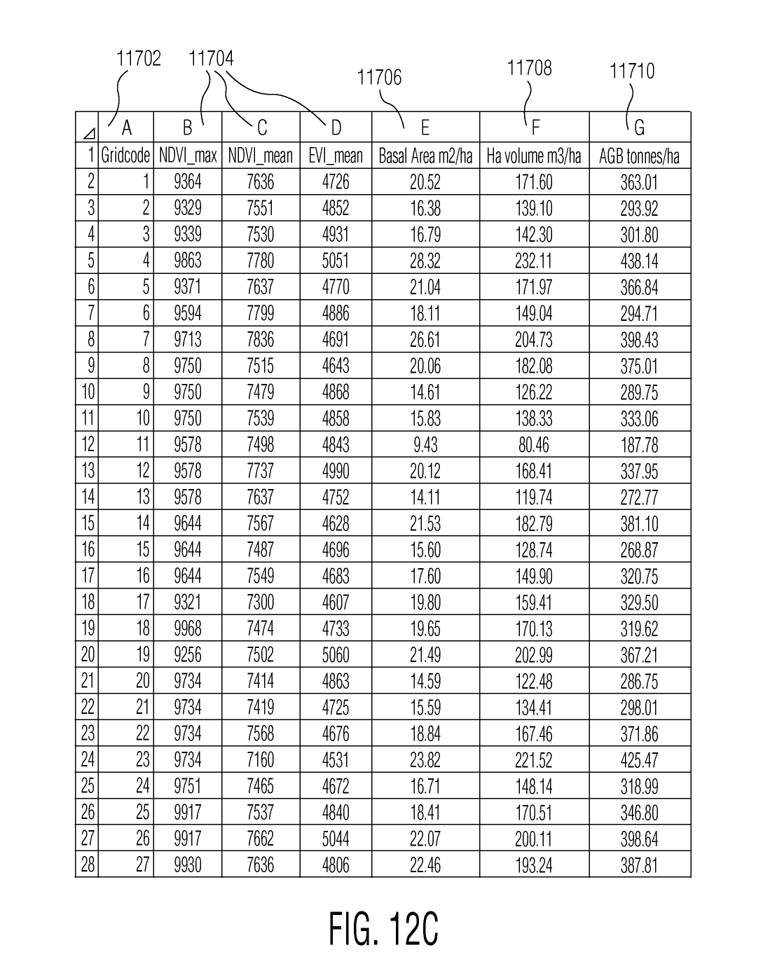

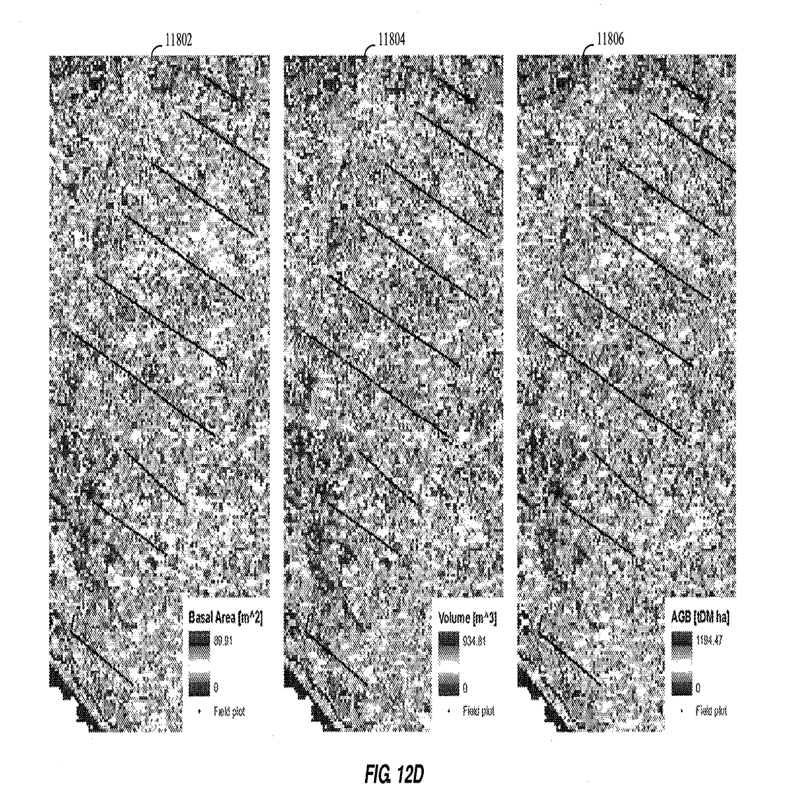

FIG. 12A shows the generic process for implementing Allometric Equations 2 with input data. A user accesses a computer workstation in 1102 that includes a screen display(s), processor(s), hard drive(s), a keyboard, a mouse, a router connected to the internet and other physical elements related to a computer workstation, etc. A computer workstation 1102 in this context can store, retrieve, process and/or output data and/or communicate with other computers. Copyright 2 in 418 is viewed in 1104 as either a printed out hard copy and/or accessed in Portable Document Format (i.e, .pdf and/or similar file format) by a computer workstation in 1102. The computer workstation is used to access: 1) data mining software in 1106, 2) the input data stored in spreadsheet format on Database 3 in 1108 and 3) the predictive model developed from the training dataset for Allometric Equations 2 stored on Database 5 in 1110. In 1112, the input data in 1108 and the training model in 1110 are loaded into the data mining software from 1106. The data mining software is processed in 1112 to evaluate and score input data from 1108 for predictions based on the training model in 1110. The outputs are saved to Database 7 as a new spreadsheet in 1114. The computer workstation is used to access geospatial data processing software in 1118. Examples of geospatial data processing software are the following: ArcView/GIS, Erdas Imagine, Envi, Idrisi, Quantum GIS, Grass, and Land Change Modeler and/or other relevant image processing software, etc., that is copyrighted and/or copyrightable and/or in open access. Database 7 is accessed in 1116, the outputs of 1112 are loaded into the geospatial data processing software in 1120 and processed to convert the scored outputs from 1112 (that are either georeferenced and/or accompanied with a grid code coordinate) to a raster file. The outputs of 1120 are stored on Database 7 in 1114. The outputs of 1120 are accessed in 1116, printed out as a spreadsheet and/or an atlas and defined as Copyright 6 in 1122 as either a Portable Document Format (i.e, .pdf and/or similar file format) digital file and/or in hard copy with a printer in 1124.

FIG. 12B shows an example for the generic process for implementing Allometric Equations 2 with the outputs of FIG. 10B and Science Plan-Directions 4. A user accesses a computer workstation in 11602 that includes a screen display(s), processor(s), hard drive(s), a keyboard, a mouse, a router connected to the internet and other physical elements related to a computer workstation, etc. A computer workstation 11602 in this context can store, retrieve, process and/or output data and/or communicate with other computers. Copyright 2 in 10418 is viewed in 11604 as either a printed out hard copy and/or accessed in Portable Document Format (i.e, .pdf and/or similar file format) by a computer workstation 11602. The computer workstation is used to access 1) Rattle in R in 11606, 2) MODIS annual mean/max NDVI and mean EVI stored in spreadsheet format stored on Database 3 in 11608 and 3) the predictive Random Forest model developed from the training dataset for Allometric Equations 2 stored on Database 5 in 11610. In 11612, the mean/max NDVI and mean EVI data in 11608 and the Random Forest training model in 11610 are loaded into Rattle. Rattle is processed in 11612 to evaluate and score the mean/max NDVI and mean EVI data from 1108 for predictions based on the Random Forest training model in 11610. The outputs are saved to Database 7 as a new spreadsheet in 11614. The computer workstation is used to access ArcGIS in 11618. Database 7 is accessed in 11616, the outputs from 11612 are loaded into ArcGIS in 11620 and processed to convert the scored outputs from 11612 (that are georeferenced and/or in grid code coordinate) to a raster file. The outputs of 11620 are stored on Database 7 in 11614. The outputs of 11620 are accessed in 11616, printed out as a spreadsheet and/or an atlas and/or an illustration and defined as Copyright 6 in 11622 in either a Portable Document Format (i.e, .pdf and/or similar file format) digital file and/or in hard copy with a printer 11624. FIG. 12C is an example of the scored outputs from 11612 in a spreadsheet with a gridcode. The gridcode coordinate per sample used as a pixel in a raster image is shown in 11702. The individual NDVI mean/max and mean EVI values per gridcode are shown in 11704. The scored outputs show the following: basal area in 11706, volume in 11708 and above ground biomass in 11710. FIG. 12D is an example of the scored outputs from 11620 as an atlas and/or an illustration reflecting the raster file for the following scored outputs: basal area in 11802, volume in 11804 and above ground biomass in 11806.

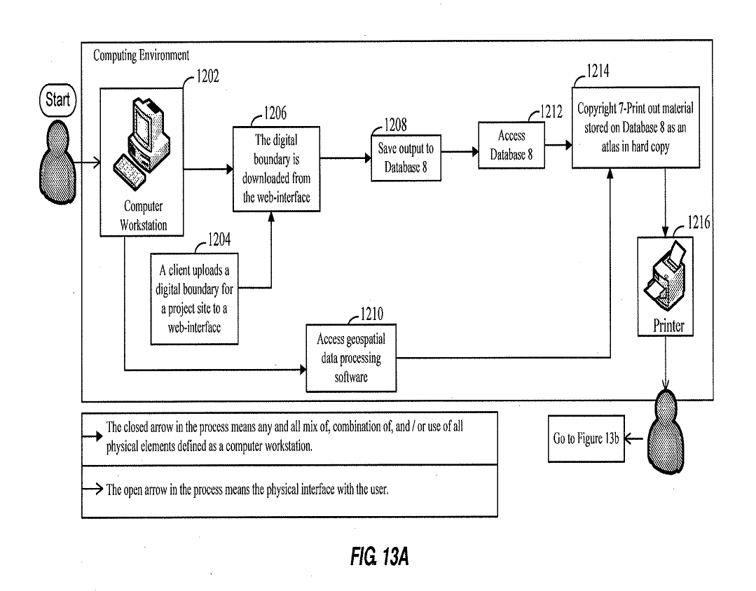

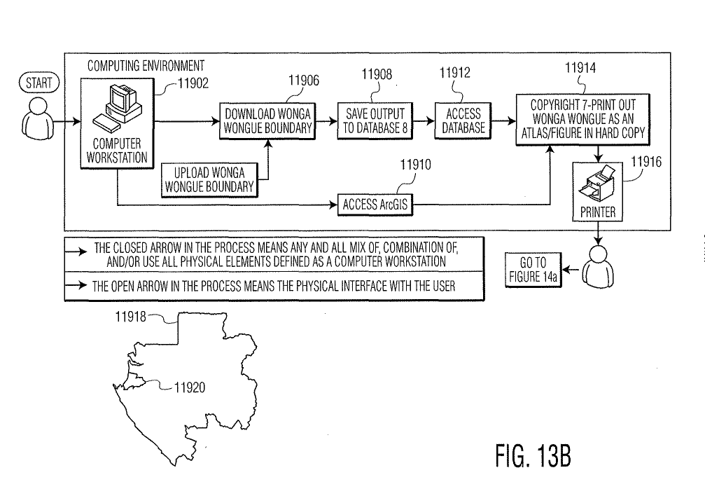

FIG. 13A shows the generic process for obtaining a project boundary from a client. A user accesses a computer workstation in 1202 that includes a screen display(s), processor(s), hard drive (s), a keyboard, a mouse, a router connected to the internet and other physical elements related to a computer workstation, etc. A computer workstation 1202 in this context can store, retrieve, process and/or output data and/or communicate with other computers. In 1204, a client uploads the digital boundary of a project site to an internet interface (i.e., via email and/or a website). In 1206, the computer workstation is used to download the digital boundary from the internet interface. The client's digital boundary is stored in Database 8 in 1208. Geospatial data processing software is accessed in 1210. Examples of geospatial data processing software are the following: ArcView/GIS, Erdas Imagine, Envi, Idrisi, Quantum GIS, Grass, and Land Change Modeler and/or other relevant image processing software, etc., that is copyrighted and/or copyrightable and/or in open access. The files stored on Database 8 are accessed in 1212 with the geospatial data processing software. The client's digital boundary is printed out as an illustration and/or atlas and defined as Copyright 7 in 1214 in either a Portable Document Format (i.e, .pdf and/or similar file format) digital file and/or in hard copy with a printer in 1216.

FIG. 13B shows an example for the generic process for obtaining a project boundary applied to the Wonga-Wongué nature reserve in Gabon. A user accesses a computer workstation in 11902 that includes a screen display(s), processor(s), hard drive(s), a keyboard, a mouse, a router connected to the internet and other physical elements related to a computer workstation, etc. A computer workstation 11902 in this context can store, retrieve, process and/or output data and/or communicate with other computers. In 11904, a client uploads the digital boundary for the Wonga-Wongué nature reserve to a web-interface (i.e., via email and/or a website). In 11906, the computer workstation in 11902 is used to download the digital boundary for the Wonga-Wongué nature reserve from the web-interface. The digital boundary for the Wonga-Wongué nature reserve is stored in Database 8 in 11908. ArcGIS is accessed in 11910. The Wonga-Wongué nature reserve boundary file stored on Database 8 is accessed in 11912 with ArcGIS. The Wonga-Wongué nature reserve boundary file is printed out as an illustration and/or atlas and defined as Copyright 7 in 11914 in either a Portable Document Format (i.e, .pdf and/or similar file format) digital file and/or in hard copy with a printer in 11916. The output of 11914 is shown in 11918, where the boundary of Wonga-Wongué nature reserve is 19920.

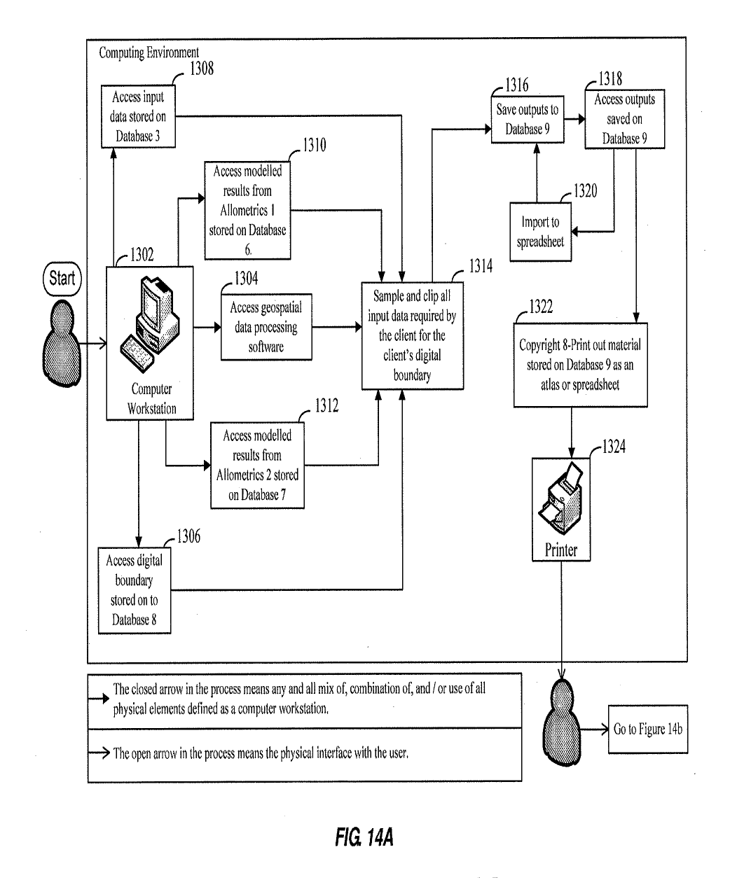

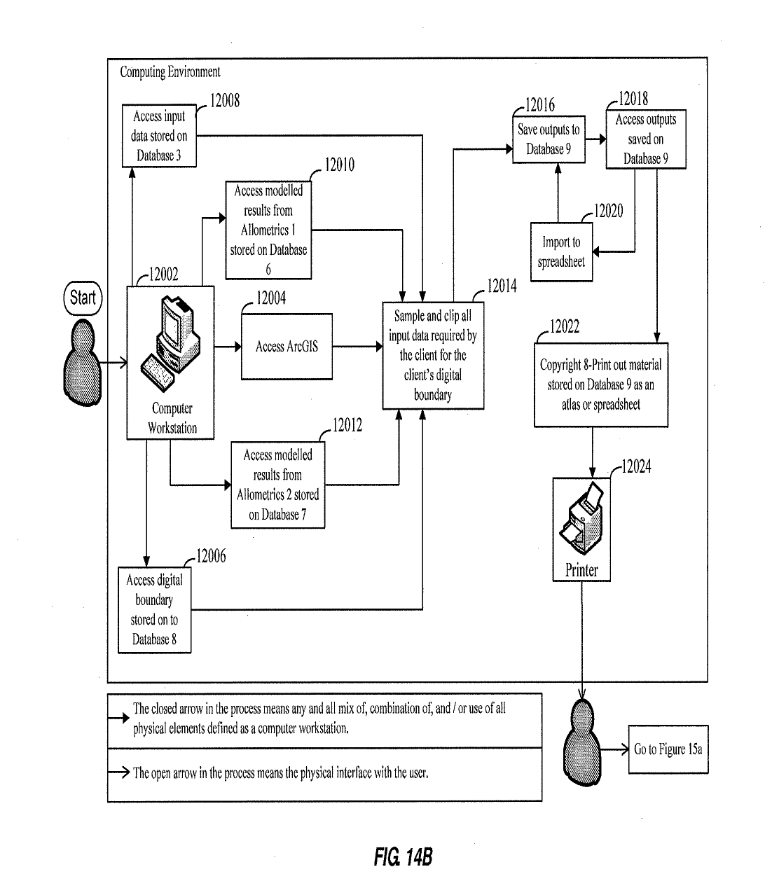

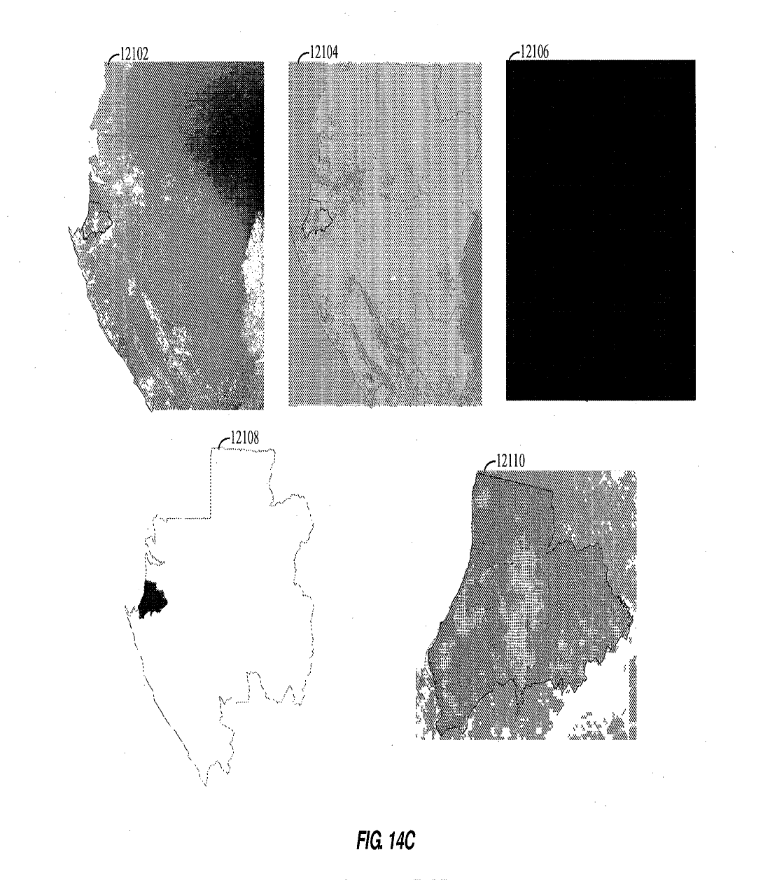

FIG. 14A shows the generic process for sampling input data and the outputs of Allometric Equations 1 and 2 with a client's project boundary. A user accesses a computer workstation in 1302 that includes a screen display(s), processor(s), hard drive(s), a keyboard, a mouse, a router connected to the internet and other physical elements related to a computer workstation, etc. A computer workstation 1302 in this context can store, retrieve, process and/or output data and/or communicate with other computers. The computer workstation is used to access any and/or all of the following: 1) a geospatial data processing software in 1304, 2) the client's digital boundary stored on Database 8 in 1306, 3) the input data stored on Database 3 in 1308, 4) the modeled results for Allometric Equations 1 stored on Database 6 in 1310, and 5) the modeled results for Allometric Equations 2 stored on Database 7 in 1312. Examples of geospatial data processing software are the following: ArcView/GIS, Erdas Imagine, Envi, Idrisi, Quantum GIS, Grass, and Land Change Modeler and/or other relevant image processing software, etc., that is copyrighted and/or copyrightable and/or in open access. In 1314, the geospatial data processing software is used to overlay the client's digital boundary of a project site from 1306 over raster input data stored on Database 3 in 1308, raster outputs for Allometric Equations 1 stored on Database 6 in 1310 and/or raster outputs for Allometric Equations 2 stored on Database 7 in 1312. The raster material accessed in 1308, 1310 and 1312 is then sampled and/or clipped for 1306 in 1314. When the raster material is sampled in 1314, the sampling can be for 1) a polygon file that is the same spatial boundary as the client's project site, 2) a raster file for land cover and/or 3) a point file (converted from a land cover raster file) intersected with a polygon file that is the same spatial boundary as the client's project site. The sampled and/or clipped outputs of 1314 are saved to Database 9 in 1316. The sampled outputs of 1314 are accessed in 1318 and loaded into a spreadsheet software (i.e., MS Excel) in 1320. The outputs of 1314 are printed out as a table and/or an atlas and/or an illustration and defined as Copyright 8 in 1322 in either a Portable Document Format (i.e, .pdf and/or similar file format) digital file and/or in hard copy with a printer in 1324.

FIG. 14B shows an example for the generic process for sampling input data and the outputs of Allometric Equations 1 and 2 with a client's project boundary applied to the Wonga-Wongué nature reserve in Gabon. A user accesses a computer workstation in 12002 that includes a screen display(s), processor(s), hard drive(s), a keyboard, a mouse, a router connected to the internet and other physical elements related to a computer workstation, etc. A computer workstation 12002 in this context can store, retrieve, process and/or output data and/or communicate with other computers. The computer workstation is used to access the following: 1) ArcGIS in 12004, 2) the Wonga-Wongué nature reserve digital boundary stored on Database 8 in 12006, and 3) the modeled results for above ground carbon flux from Allometric Equations 1 stored on Database 6 in 12010. In 12014, ArcGIS is used to overlay the Wonga-Wongué nature reserve digital boundary (as a polygon shapefile [i.e., .shp and associated files]) from 12006 over the modeled results for the above ground carbon flux raster file in 12010. The above ground carbon flux raster file in 12010 is then sampled and/or clipped for Wonga-Wongué nature reserve digital boundary in 12014. FIG. 14C shows illustrative examples of the sampling from 12014 in greater detail. In 12102, the polygon file for Wonga-Wongué nature reserve is overlaid on above ground biomass flux. The polygon is used to sample for above ground biomass flux of the gridded pixels cells within the Wonga-Wongué nature reserve boundary. A land cover raster map classified to AFOLU classes (see Section 1.22 in 10418) is shown under the Wonga-Wongué nature reserve polygon file in 12104. ArcGIS is used to convert the land cover raster map in 12104 to a point shape file in 12106, where individual points have the same numerical classification value as the land cover map. ArcGIS is used to intersect the point file in 12106 with the polygon file of Wonga-Wongué in 12108. In 12010, the point file from 12108 is overlaid on the above ground biomass flux rater file which is sampled by the land cover numerical values in 12106. Returning to FIG. 14C, the sampled and/or clipped outputs of 12014 are saved to Database 9 in 12016. The sampled outputs of 12014 are accessed in 12018 and loaded into spreadsheet software (i.e., MS Excel) in 12020. The outputs of 12014 are printed out as a table and/or an atlas and/or an illustration and defined as Copyright 8 in 12022 in either a Portable Document Format (i.e, .pdf and/or similar file format) digital file and/or in hard copy with a printer in 12024. Table 51a shows as example of the outputs from above ground biomass flux sampled for the Wonga-Wongué nature reserve polygon and 1b shows as example of the outputs from above ground biomass flux sampled for the Wonga-Wongué nature reserve point file representing land cover classes. FIG. 14C is indicative of the atlas/illustration output of 12022.

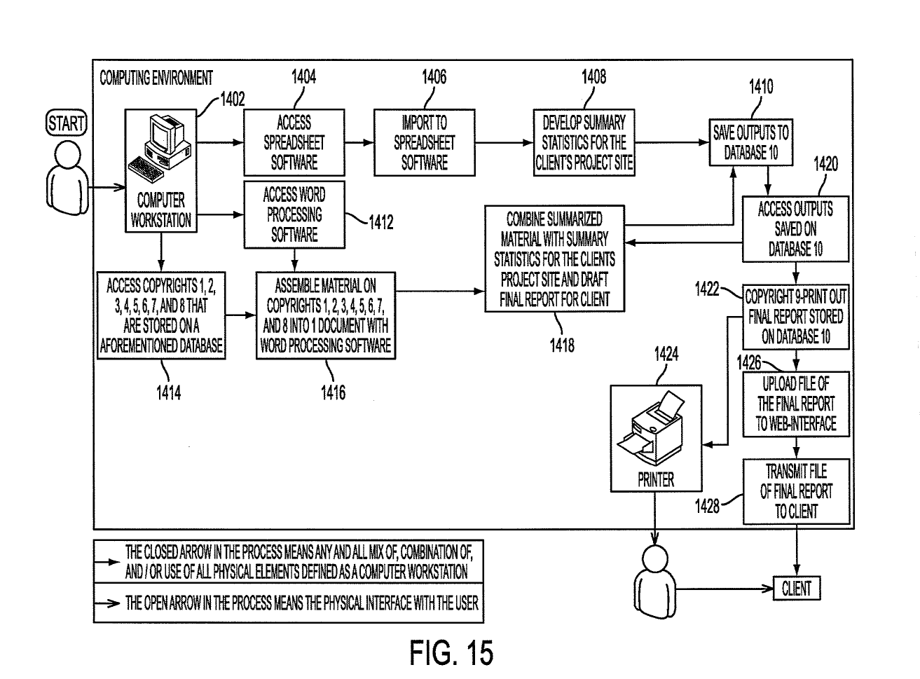

FIG. 15 shows the generic process for reporting information to the client. A user accesses a computer workstation in 1402 that includes a screen display(s), processor(s), hard drive (s), a keyboard, a mouse, a router connected to the internet and other physical elements related to a computer workstation, etc. A computer workstation 1402 in this context can store, retrieve, process and/or output data and/or communicate with other computers. The computer workstation is used to access a spreadsheet software (i.e, MS Excel) in 1404. In 1406, the sampled data stored in spreadsheets on Database 9 is imported to the spreadsheet software in 1404. In 1408, summary statistics are calculated for sampled data at the client's project site. The summary statistics in 1408 include any and/or all of the following: 1) reporting the sampled data and the conversion to relevant annual GHGs and/or other vegetation attributes required in reporting at the project site; 2) development of a baseline for annual GHG emissions and removals at the project site for a period in time prior to project implementation; 3) predicted future annual GHG emissions and removals from the baseline for the project lifetime (i.e., the period in time in which the client's project will be implemented) without the project taking place; and 4) predicted future annual GHG emissions and removals for the project lifetime (i.e., the period in time in which the client's project will be implemented) with the project taking place; 5) assessment of the potential annual GHG offset from the project for the project lifetime; 6) an assessment of annual GHG emissions, removals and other relevant vegetation attributes for the period after the project was implemented. Table 5 2a and 2b shows examples above ground biomass flux for the Wonga-Wongué nature reserve converted from tonnes of carbon to tonnes of carbon dioxide used as one variable reported to the client. The summary statistics are saved to Database 10 in 1410. A computer workstation is used to access 1) a word processing software in 1412, and 2) Copyrights 1, 2, 3, 4, 5, 6, 7 and 8 that are stored on their respective databases. In 1416, the Copyrighted material from 1412 is assembled into one document with the word processing software. In 1418, the final report is drafted by combining the assembled material from 1416 with the spreadsheet for summary statistics at the project site in 1408 accessed by 1420. A review explaining the sampled summary statistics is also drafted in 1418. The outputs of 1418 are stored on 1410. The outputs of 1418 are accessed in 1420, printed out as Copyright 9 in 1422 in either a Portable Document Format (i.e, .pdf and/or similar file format) digital file and/or in hard copy with a printer in 1424. When 1422 is printed out in a Portable Document Format (i.e, .pdf and/or similar file format) digital file, the file is uploaded to an internet interface (i.e., email and/or a web-site) in 1426. The uploaded material in 1426 is then transmitted electronically via the internet to the client in 1428.

TABLE 5

| | MIN | MAX | RANGE | MEAN | STD | SUM |
|---|---|---|---|---|---|---|
| ΔC_{AB} | (tC ha⁻¹) | (tC ha⁻¹) | (tC ha⁻¹) | (tC ha⁻¹) | (tC ha⁻¹) | (tC) |
| 1a. Polygon | | | | | | |
| Wonga-Wongué | 0.00 | 0.46 | 0.46 | 0.20 | 0.10 | 98,496.39 |
| Nature Reserve | | | | | | |
| 1b. Point | | | | | | |
| Forested land | 0.00 | 0.38 | 0.38 | 0.25 | 0.04 | 92,905.83 |
| Grassland | 0.00 | 0.46 | 0.46 | 0.05 | 0.08 | 4799.70 |
| Cropland | 0.00 | 0.01 | 0.01 | 0.01 | 0.00 | 8.00 |
| Wetland | 0.00 | 0.29 | 0.29 | 0.17 | 0.07 | 366.90 |
| | MIN | MAX | RANGE | MEAN | STD | SUM |
| ΔC_{AB} | (tCO₂ ha⁻¹) | (tCO₂ ha⁻¹) | (tCO₂ ha⁻¹) | (tCO₂ ha⁻¹) | (tCO₂ ha⁻¹) | (tCO₂) |
| 2a. Polygon | | | | | | |
| Wonga-Wongué | 0.00 | 1.69 | 1.68 | 0.75 | 0.37 | 361,153.44 |
| Nature Reserve | | | | | | |
| 2b. Point | | | | | | |
| Forested land | 0.01 | 1.39 | 1.38 | 0.92 | 0.15 | 340,654.72 |
| Grassland | 0.00 | 1.69 | 1.68 | 0.17 | 0.30 | 17,598.89 |
| Cropland | 0.01 | 0.03 | 0.01 | 0.02 | 0.00 | 29.33 |
| Wetland | 0.01 | 1.07 | 1.06 | 0.61 | 0.27 | 1345.30 |

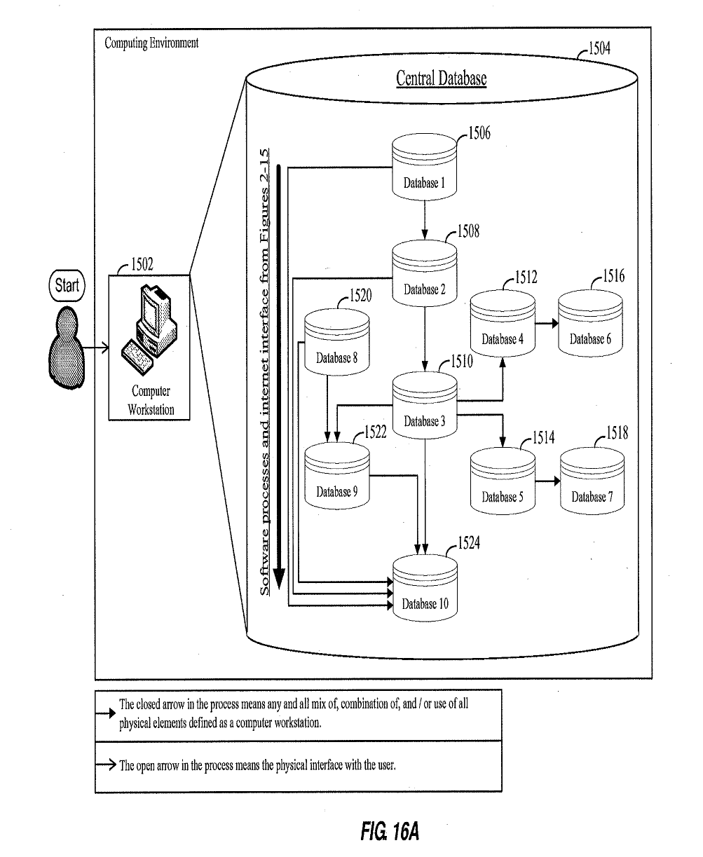

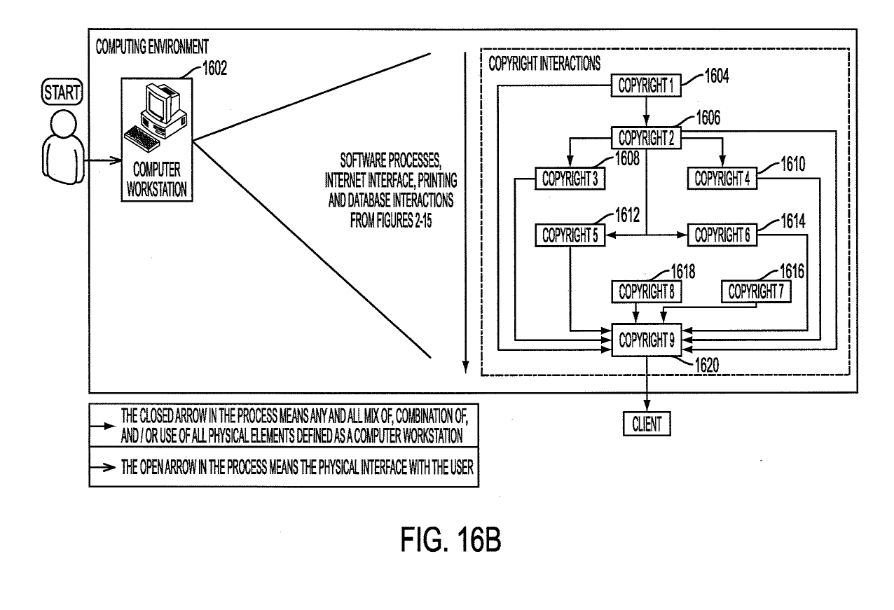

FIG. 16A shows the initial key interactions and structure of Database 1 through Database 10. A user accesses a computer workstation in 1502 that includes a screen display(s), processor(s), hard drive(s), a keyboard, a mouse, a router connected to the internet and other physical elements related to a computer workstation, etc. A computer workstation 1502 in this context can store, retrieve, process and/or output data and/or communicate with other computers. In 1504, the Central Database is shown storing Database 1 through Database 10. Software processing and internet-interface from FIG. 2 through FIG. 15 are used to develop all aforementioned content stored on Database 1 through Database 10 of the Central Database. The aforementioned content of Database 1 in 1506 is developed with the software processes and internet interface described herein by FIGS. 2A-2C. The aforementioned contents of Database 1 are used to develop the aforementioned contents in Database 2 in 1508 with software processes and internet interface described herein by FIGS. 4A-4C. The aforementioned contents of Database 2 are used to develop the aforementioned contents in Database 3 in 1510 software processes and internet interface described herein by FIG. 6A and FIG. 7A.

The aforementioned contents of Database 4 in 1512 are developed within Database 3 with the aforementioned contents of Database 3 and Database 2 and the software processes described herein by FIG. 8A. The aforementioned contents of Database 5 in 1514 are developed within Database 3 with the aforementioned contents of Database 3 and Database 2 and the software processes described herein by FIG. 9A and FIG. 10A. The aforementioned contents of Database 6 in 1516 are developed within Database 4 with the aforementioned contents of Databases 2, 3 and 4 and software processes described herein by FIG. 11A. The aforementioned contents of Database 7 in 1518 are developed within Database 5 with the aforementioned contents of Databases 2, 3 and 5 and the software processes described herein by FIG. 12A. The aforementioned contents of Database 8 in 1520 are developed with software processes and internet interface described herein by FIG. 13A. The aforementioned contents of Database 9 in 1522 are developed with the aforementioned contents of Databases 3, 6, 7 and 8 and software processes described herein by FIG. 14A. The aforementioned contents of Database 10 in 1524 are developed with the aforementioned contents of Databases 1, 2, 3, 4, 5, 6, 7, 8, and 9 and software processes described herein by FIG. 15. All aforementioned database content, interactions, software processes and internet interface within the Central Database are non-limiting within the scope of the aforementioned conceptual material and can be changed, combined, mixed, added to, adapted, updated, restructured, renamed, revised, retrieved, evolved and/or stored on a meta-database within Central Database.

FIG. 16B shows the full assembly line for creating the final output of Copyright 9. A user accesses a computer workstation in 1602 that includes a screen display(s), processor(s), hard drive(s), a keyboard, a mouse, a router connected to the internet and other physical elements related to a computer workstation, etc. A computer workstation at 1602 in this context can store, retrieve, process and/or output data and/or communicate with other computers. The process starts in 1604 with Copyright 1 for the legal/policy review developed as the output from the generic process of FIG. 2A in 238. Copyright 1 is used to develop Copyright 2 for a science plan from the outputs of the generic process of FIG. 4C in 418. Copyright 2 in 1606 is used to inform the development and implementation of Allometric Equations 1 (in 1608 for Copyright 3 and in 1612 for Copyright 5) and Allometric Equations 2 (in 1610 for Copyright 4 and in 1614 for Copyright 6). FIG. 8A shows the generic process used to develop Copyright 3 as the output for 720. FIGS. 9A and 10A show the generic process used to develop Copyright 4 from developing Allometric Equations 2 as the output in 918. FIG. 11A shows the generic process used to develop Copyright 5 as output from 1018 from implementing Allometric Equations 1 with remote sensing imagery. FIG. 12A shows the generic process used to develop Copyright 6 as output in 1122 from implementing Allometric Equations 2 with remote sensing imagery. FIG. 13A shows the generic process used to develop Copyright 7 in 1214. FIG. 14A shows the generic process used to obtain Copyright 8 from the outputs in 1322. Copyright 7 in 1616 and Copyright 8 in 1618 are not directed linked to other copyrights. In 1620, all copyrights are assembled into the final document as Copyright 9. The assembly is completed with a text retrieval software that organizes the copyrights in one document in ascending order by Copyright number. FIG. 15A shows the generic process used to develop Copyright 9 with outputs in 1422 that are electronically transmitted to the client via the internet.

In summary, the full invention relates to a computer implemented system typically with eights steps that are used to monitor and report relevant greenhouse gases for an offset project. However, the number of steps is not limited to eight, one or more of the steps can be combined, omitted, performed in any order according to application criteria. For example, the eight steps are referenced in FIG. 1 and are the following: 1) a method for developing a legal/policy analysis; 2) a method for developing a science plan based on the legal/policy analysis, 3) the development of a geospatial database based on the science plan; 4) a method for developing an allometric model that is based on the science plan and geospatial database; 5) a method for implementing the allometric equations with remote sensing imagery based on the science plan and geospatial database; 6) a method for obtaining a client's geographical boundary of an offset through an internet interface; 7) a method for sampling a client's geographical boundary for the contents of the geospatial database, 8) a method for developing a report from the outputs of steps 1-7 that is transmitted to the client through an internet interface.

The first step of the full invention relates to a method for developing a legal/policy review for a target greenhouse gas. The full method for developing a legal/policy analysis is shown in FIG. 2A. The first step in the method for developing a legal/policy analysis includes developing a database for any and all relevant policy documents related to mitigating climate change. The second step in the method for developing a legal/policy analysis includes using text retrieval software to search for key words on any relevant policy document stored on the policy document database. FIG. 2B shows the automated method to search and retrieve text, figures and tables from policy documents. The retrieved information is used to compile parameters for monitoring a target greenhouse gas. The key words are stored on a meta-database. The third step in the method for developing a legal/policy analysis includes structuring the policy documents into tiers based on legal priority. FIG. 2C shows a tiered structure to assess policy documents by legal priority. The policy documents are then compared for monitoring guidance requirements between documents at different tiers. The comparison assesses whether the monitoring requirements for each tier is fungible with a tier that has greater legal priority.

The second step of the full invention relates to a method for developing a science plan based on the outputs of the legal/policy analysis. The full method for developing a science plan is shown in FIG. 4D. The first step in the method to develop a science plan includes developing a database on current and planned satellite missions and remote sensing instruments. The second step in the method for developing a science plan includes using text retrieval software to search for key words that describe the remote sensing instrument's monitoring capabilities for vegetation. The text retrieval is performed on the database of current and planned satellite missions and remote sensing instruments. FIG. 4A shows the automated method to search and retrieve information from the satellite mission and remote sensing instrument. The third step in the method for developing a science plan includes an output from the assessment of the remote sensing database. The output assesses current and future satellite missions and remote sensing instruments in relation to the data continuity requirements for the lifetime of a client's project activity. FIG. 4B shows an example of the output from the assessment of current and planned satellite missions and remote sensing instruments in relation to the lifetime of an offset project. The point of the output is that there must be overlapping data continuity between satellite missions to monitor a offset project. The fourth step in the method for developing a science plan includes developing a database of peer-reviewed journal for the remote sensing instrument that best meets the data continuity requirements for an offset project. The fifth step in the method for developing a science plan includes using text retrieval software to search for key words in the peer-reviewed journal articles that describe key words developed by the legal/policy review. FIG. 4C shows the automated method to search and retrieve information from the peer-reviewed journal articles. The sixth step in the method for developing a science plan includes directions used to monitor an offset project. The directions are developed from the information retrieved from peer-reviewed journal articles. The directions 1) define the current knowledge space in public access that does not explicitly monitor the target greenhouse gas and 2) defines new knowledge space that explicitly monitors the target greenhouse gas for the outputs from the legal/policy analysis. Examples of directions are found in FIGS. 5A-5D. These are examples of the directions used that are used to 1) develop the allometric equations and 2) implement the allometric equations with remote sensing imagery. These examples of directions explain two methods to an develop allometic model: 1) Allometric Equations 1 that use a process-based dynamic ecosystem model to develop fractional functions that will be implemented with remote sensing imagery and 2) Allometric Equations 2 that will be used to develop regression and/or classification functions between physical samples of a vegetation attribute and samples from remote sensing imagery.

The third step of the full invention relates to developing a geospatial database from the science plan. The method for developing a geospatial database is shown in FIG. 6A. The geospatial database consists of 1) free geospatial data, 2) geospatial data that is not free, but can be purchased from a provider and/or, and 3) the geospatial data obtained from a client through an internet interface. The geospatial database includes the following: a standard remote sensing imagery product that fulfills data continuity requirements for monitoring a vegetation attribute within the geographical boundaries of the offset project; a secondary remote sensing imagery product at a higher resolution than the standard remote sensing imagery product, but with fewer replicates over time than the standard remote sensing imagery product; climate geospatial data; elevation geospatial data; soil geospatial data; vegetation attribute geospatial data; peer-review literature and trading mechanism reports containing a geospatial reference to vegetation attributes; and/or official government disclosures for vegetation attributes with a geospatial references and/or disclosures of geospatial data for a measurement of a vegetation attribute. The contents of the geospatial database are preprocessed in relation to the contents of the science plan. FIG. 7A shows an example for the preprocessing the raw downloaded remote sensing imagery with a geospatial data processing software to: 1) sub-set any condensed data files, 2) remove poor quality pixel information in the remote sensing imagery, 3) develop qualitative statistics (mean, max, min, etc) for any period in time that the database encompasses. The remote sensing imagery can also be preprocessed by any mixing and/or combining of different remote sensing images and/or bands to create an index of multiple remote sensing images. The preprocessing of the vegetation attribute geospatial data can include converting any and/or all vegetation attribute geospatial data into one consolidated vector file format. The vegetation attribute information stored on the database related peer-reviewed journal articles and/or trading mechanism reports containing a geographically referenced coordinate for measurements of vegetation attributes can be processed with text retrieval software to extract the information related to vegetation attribute(s) and the georeferenced coordinate.

The fourth step of the full invention relates to a method(s) for developing an allometric model(s) based on the outputs of the science plan and the contents of the geospatial database. Allometric Equations 1 are developed with a process-based dynamic ecosystem model for fractions. FIG. 8A shows the method of developing fractions with a process-based dynamic ecosystem model. The method uses a processed-based dynamic ecosystem model and input data stored on the geospatial database to develop fractions based on the directions in the science plan. Allometric Equations 2 are developed for regression/classification functions between physical sample of a vegetation attribute and samples from remote sensing imagery with a data mining software. The process includes a method to extract the geospatial information in a pixel of a remote sensing image that is at the same geographical coordinate as the geospatial data of the vegetation attribute. FIG. 9A shows a method for extracting remote sensing data in a raster file by a point vector file. A geospatial data processing software is used to extract the remote sensing data. The method for extracting the data includes input data from the geospatial database, including 1) geospatial data for a vegetation attribute that is in a point vector file and 2) remote sensing imagery that is in a rater file. The next step in the process is a method of developing a training model between the samples of the vegetation attribute and the samples from the remote sensing imagery with data mining software. FIG. 10A shows a method of developing an allometric model between a physical sample of a vegetation attribute and a sample from remote sensing imagery. The method for developing the training model uses a sample for a vegetation attribute and the sample for remote sensing imagery to develop a regression and/or a classification functions between the two samples.

The fifth step of the full invention relates to a method(s) for implementing an allometric model(s) with remote sensing imagery based on the outputs of the science plan and the contents of the geospatial database. Allometric Equations 1 use the fractional function outputs for Allometric Equations 1 developed in the fourth step with input geospatial data. The input geospatial data is for standard remote sensing products of a vegetation attribute, for example MODIS MOD 17 Net Primary Production product (NPP). The implementation is completed with a geospatatial data processing software. FIG. 11A shows a method for processing the outputs of Allometic Equations 1 with remote sensing imagery. The output is a new map of geospatial data for the specific vegetation attribute defined by the directions in the science plan that meets the requirements for monitoring and/or reporting in the legal/policy analysis. Allometric Equations 2 use the regression and/or classification function outputs for Allometric Equations 2 developed in the fourth step with the full remote sensing imagery that was used as a sample when regression and/or classification function was developed. The process includes scored the information in the remote sensing imagery based on the regression and/or classification function in a data mining software. After the remote sensing imagery is scored, the scored outputs are processed with a geospatial data processing software to convert the scored outputs into a map. FIG. 12A shows a method for processing the outputs Allometric Equations 2 with data mining software and geospatial data processing software. The output is a new map of geospatial data for the specific vegetation attribute defined by the directions in the science plan that meets the requirements for monitoring and/or reporting in the legal/policy analysis. The outputs of Allometric Equations 1 and 2 are stored on the geospatial database.

The sixth step of the full invention relates to a method(s) for obtaining a geospatial boundary vector file from a client. FIG. 13A shows a method for obtaining a geospatial boundary of an offset project from a client through an internet interface. The interface is either by email and/or a web-site.

The seventh step of the full invention relates to a method(s) for sampling the client's geospatial boundary vector file for any of the contents stored on the geospatial database. FIG. 14A shows a method for sampling a client's geospatial boundary vector file with any of the following: 1) any geospatial contents stored on the geospatial database; and/or 2) the outputs of Allometric Equations 1; and/or 3) the outputs of Allometric Equations 2. The sampling is completed with geospatial data processing software.

The seventh step of the full invention relates to a method(s) for developing and submitting a final report to a client. The report is the assembly of all outputs from steps 1-7 of the full invention. FIG. 15 shows a method for developing a final report and transmitting the final report to a client. The final report is transmitted to a client via an internet interface.

FIG. 16A shows a method of database interactions from database 1 through 10 that occurs as a result of the software processes and internet interface from steps 1-8 of the full process. All contents stored on databases 1-10 are stored on a central database. FIG. 16B shows the automated assembly line of Copyrights 1-9 that is implemented with text retrieval software. The final out defined as Copyright 9 is transmitted to a client through an internet interface.

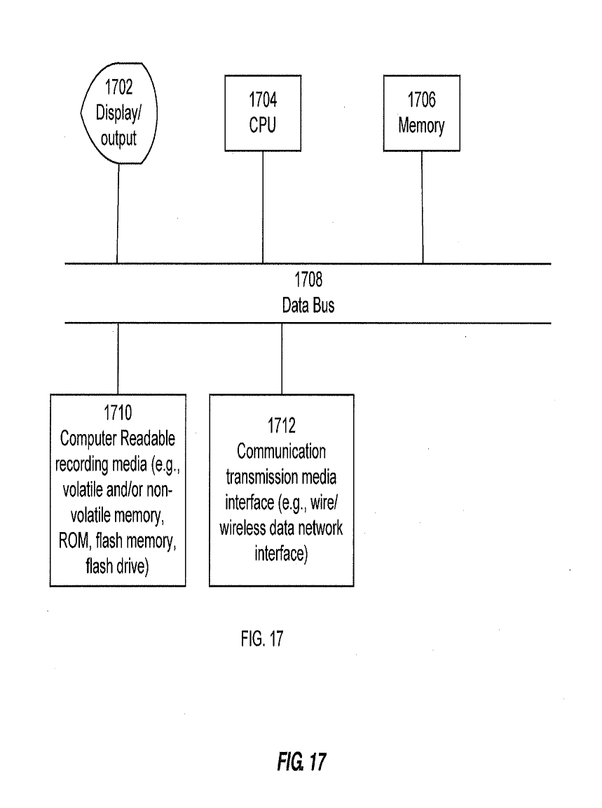

FIG. 17 is a functional block diagram of a computer for the embodiments of the invention, namely the computer is an example of a computer workstation and/or client/server in which the embodiments can be implemented. In FIG. 17, the computer can be any computing device. Typically, the computer includes a display or output unit 1702 to display a user interface or output information or indications, such as a diode. A computer controller 1704 (e.g., a hardware central processing unit) executes instructions (e.g., a computer program or software) that control the apparatus to perform operations. Typically, a memory 1706 stores the instructions for execution by the controller 1704. According to an aspect of an embodiment, the apparatus reads/writes/processes data of any computer readable recording media 1710 and/or communication transmission media interface 1712. The display 1702, the CPU 1704 (e.g., hardware logic circuitry based computer processor that processes instructions, namely software), the memory 1706, the computer readable media 1710, and the communication transmission media interface 1712, are in communication by the data bus 1708. Any results produced can be output, for example, printed or displayed on a display for the computing hardware.

According to an aspect of the embodiments of the invention, any combinations of one or more of the described features, functions, operations, and/or benefits can be provided. A combination can be one or a plurality. The phrase ‘all’ includes and can be one or more, or all, or any combinations. The embodiments can be implemented as an apparatus (a machine) that includes computing hardware (i.e., computing apparatus), such as (in a non-limiting example) any computer that can store, retrieve, process and/or output data and/or communicate (network) with other computers. According to an aspect of an embodiment, the described features, functions, operations, and/or benefits can be implemented by and/or use computing hardware and/or software. The apparatus (e.g., the computer workstations, servers, etc. can comprise a controller (CPU) (e.g., a hardware logic circuitry based computer processor that processes or executes instructions, namely software/program), computer readable media, transmission communication interface (network interface), and/or an output device, for example, a display device, all in communication through a data communication bus. In addition, an apparatus can include one or more apparatuses in computer network communication with each other or other apparatuses. In addition, a computer processor can include one or more computer processors in one or more apparatuses or any combinations of one or more computer processors and/or apparatuses. An aspect of an embodiment relates to causing one or more apparatuses and/or computer processors to execute the described operations. The results produced can be output to an output device, for example, displayed on the display.

A program/software implementing the embodiments may be recorded on a computer-readable media, e.g., a non-transitory or persistent computer-readable medium. Examples of the non-transitory computer-readable media include a magnetic recording apparatus, an optical disk, a magneto-optical disk, and/or volatile and/or non-volatile semiconductor memory (for example, RAM, ROM, etc.). Examples of the magnetic recording apparatus include a hard disk device (HDD), a flexible disk (FD), and a magnetic tape (MT). Examples of the optical disk include a DVD (Digital Versatile Disc), DVD-ROM, DVD-RAM (DVD-Random Access Memory), BD (Blue-ray Disk), a CD-ROM (Compact Disc-Read Only Memory), and a CD-R (Recordable)/RW. The program/software implementing the embodiments may be transmitted over a transmission communication path, e.g., a wire and/or a wireless network implemented via hardware. An example of communication media via which the program/software may be sent includes, for example, a carrier-wave signal.

The many features and advantages of the embodiments are apparent from the detailed specification and, thus, it is intended by the appended claims to cover all such features and advantages of the embodiments that fall within the true spirit and scope thereof. Further, since numerous modifications and changes will readily occur to those skilled in the art, it is not desired to limit the inventive embodiments to the exact construction and operation illustrated and described, and accordingly all suitable modifications and equivalents may be resorted to, falling within the scope thereof.

What is claimed is:

1. A method of monitoring the effectiveness of a target greenhouse gas offset activity within a geographical boundary of an offset project, comprising:
the use of text retrieval software and/or search technology to key word retrieve/search for relevant information on monitoring and/or reporting of a vegetation attribute from one or more target policies to compile policy parameters for the target greenhouse gas;
use of text retrieval software and/or search technology to key word retrieve/search for generating directions for monitoring the target greenhouse gas offset activity within the target geographical boundary of the offset project, to generate a science plan for monitoring the effectiveness of target greenhouse gas offset activity for the target geographical boundary of the offset project, based upon the compiled policy parameters;
generating a geospatial database including remote sensing imagery for monitoring the target greenhouse gas offset activity within the target geographical boundary of the offset project that is based on the generated science plan;
generating an allometric model for the target greenhouse gas offset activity within the geographical boundary of the offset project, based upon the science plan for monitoring the target greenhouse gas offset activity for the target geographical boundary and the contents of the geospatial database;
the generation of the allometric model includes one or more functions of fractions, regressions, and/or classifications between the contents of the geospatial database and the target greenhouse gas within the target geographic boundary of the offset project; and
generating new geospatial data which predicts the effectiveness of the target greenhouse gas offset activity within the target geographical boundary of the offset project based upon the policy parameters, science plan and the allometric model,
wherein the generated geospatial data which predicts the effectiveness of target greenhouse gas offset activity is based upon one or more measurements of a vegetation attribute within the target geographical boundary of the offset project and upon mapped geospatial outputs of the allometric model of fractions, regressions, and/or classifications,
wherein the allometric model of fractions relate a biophysical element of a vegetation attribute to another biophysical element of a vegetation attribute, and the allometric model of regression and/or classification functions relate a physical measurement of a vegetation attribute to digital information of another vegetation attribute measurable in pixels of remote sensing imagery,
and wherein the allometric functions of regressions and/or classifications are based upon a physical sample for a measurement of a vegetation attribute that has a geographical coordinate and a sample of pixels from remote sensing imagery that are at the same or a similar geographical coordinate as the physical sample of the vegetation attribute.

2. The method according to claim 1, wherein the allometric function of a fraction based upon a measurement of a vegetation attribute is generated by processing a dynamic ecosystem model with input data, based upon the directions in the science plan.

3. The method according to claim 2, wherein geospatial data processing software is used to implement the allometric function of the fraction with remote sensing imagery of another vegetation attribute, based upon the directions in the science plan.

4. The method according to claim 3, wherein an output of the geospatial data processing software is a map of the target vegetation attribute.

5. The method according to claim 1, wherein the allometric function of a regression is generated by data mining software with a function based upon a physical sample of a target vegetation attribute and a pixel sample from remote sensing imagery.

6. The method according to claim 1, wherein the allometric function of a classification is generated by data mining software with the function based upon a physical sample of the target vegetation attribute and a pixel sample from remote sensing imagery.

7. The method according to claim 1, wherein the allometric model generated from the functions of regressions and/or classifications are used as a predictor model in data mining software to score any and/or all pixels in the remote sensing imagery that was used to develop the regression and/or classification function with a target vegetation attribute.

8. The method according to claim 7, wherein the output from the scored pixels from the data mining software are processed in a geospatial data processing software to create a map of the target vegetation attribute.

9. The method according to claim 1, wherein the geospatial data for the target geographical boundary of the offset project is obtained through an internet interface.

10. The method according to claim 1, wherein the target measurement of a vegetation attribute within the target geographical boundary includes processing in geospatial data processing software mapped outputs of the allometric models of fractions, regressions, and/or classifications for the geospatial data of the target geographical boundary of the offset project.

11. The method according to claim 10, wherein the geospatial data processing software includes processing the mapped outputs of the allometric model for the geospatial data of the target geographical boundary that is manifested as a polygon vector file for the target boundary and/or a point vector file for the target boundary and/or pixels in a raster file for the target boundary.

12. The method according to claim 1, wherein the measurement of the one or more vegetation attributes includes a numerical biophysical element and/or a land classification element.

13. The method according to claim 1, wherein the target greenhouse gas is one or more carbon based chemical elements.

14. The method according to claim 1 wherein the generating of a geospatial database including remote sensing imagery is based upon generating a database describing current and planned satellite missions and sensor instruments.

15. The method according to claim 14, wherein the generating of a timeline is developed from the database for current and planned satellite missions and sensor instruments; the generating of the timeline for current and planned remote sensing instrument(s) includes the identification of the current and planned remote sensing instrument(s) that best fulfills the data continuity requirements for monitoring a vegetation attribute within the geographical boundaries of an offset project; and
the timeline for current and planned remote sensing instrument(s) that best fulfills the data continuity requirements for monitoring a vegetation attribute within the geographical boundaries of an offset project is used to specify which remote sensing imagery is used to generate in the geospatial database.

16. The method according to claim 14, wherein text retrieval software is used to identify specific satellite missions and instruments that have an application to monitoring a vegetation attribute within the geographical boundaries of the offset project, based upon the database describing current and planned satellite missions and sensor instruments.

17. The method according to claim 1, wherein the generating of a geospatial database from a science plan includes one or more of:
a standard remote sensing imagery product that fulfills data continuity requirements for monitoring a vegetation attribute within the geographical boundaries of the offset project, a secondary remote sensing imagery product at a higher resolution than the standard remote sensing imagery product, but with fewer replicates over time than the standard remote sensing imagery product, climate geospatial data, elevation geospatial data, soil geospatial data, and
vegetation attribute geospatial data, peer-review literature and/or trading mechanism reports containing a geospatial reference to vegetation attributes, and/or official government disclosures for vegetation attributes with a geospatial references and/or disclosures of geospatial data for a measurement of a vegetation attribute.
